(19) United States
(12) Patent Application Publication
(10) Pub. No.: US 2023/0131111 A1
(43) Pub. Date: Apr. 27, 2023

(54) BIODEGRADABLE POLYIMIDAZOLIUMS AND OLIGOIMIDAZOLIUMS

(71) Applicant: Nanyang Technological University, Singapore (SG)

(72) Inventors: Bee Eng Mary CHAN, Singapore (SG); Surendra Hittanahalli MAHADEVEGOWDA, Singapore (SG); Wenbin ZHONG, singapore (SG); Mallikharjuna Rao LAMBU, Singapore (SG); Jianghua LI, Singapore (SG); Zhangyong SI, Singapore (SG)

(21) Appl. No.: 17/926,038

(22) PCT Filed: May 25, 2021
(86) PCT No.: PCT/SG2021/050290

§ 371 Date: Nov. 17, 2022

(30) Foreign Application Priority Data
May 26, 2020 (SG) 10202004902P

(51) Int. Cl. A61K 31/787; C08G 73/06; A01N 43/50; A01P 1/00; A61P 31/04; A61P 11/00; C07D 233/61

(52) U.S. Cl. CPC A61K 31/787; C08G 73/0616; A01N 43/50; A01P 1/00; A61P 31/04; A61P 11/00; C07D 233/61

(57) ABSTRACT

Disclosed herein are compounds in the form of polymers, oligomers and defined molecules having repeating units that all incorporate repeating units formed from an imidazolium group and a biodegradable chain connected to an adjacent repeating unit. The compounds disclosed herein may have antimicrobial activity and so may be used to treat microbial infection and/or to treat surfaces to prevent microbial infections. Also disclosed herein are methods of forming the compounds.

### FIELD OF INVENTION

The current invention relates to the field of poly- and oligo-imidazoliums, as well as defined molecules having similar features. These molecules all contain degradeable (particularly biodegradable) moieties that enable them to be broken up in vivo. These molecules may be useful for the treatment of microbial infection or act as antimicrobial agents (e.g. in personal care products or on a surface).

### BACKGROUND

The listing or discussion of a prior-published document in this specification should not necessarily be taken as an acknowledgement that the document is part of the state of the art or is common general knowledge.

The emergence and spread of multidrug-resistant (MDR) pathogens are a matter of great concern to the world. Recently, the World Health Organization (WHO) made a call for action for the development of new antibacterial agents for the most problematic superbugs that include carbapenem-resistant Acinetobacter baumannii (CRE-AB), carbapenem-resistant Pseudomonas aeruginosa (CRE-PA), and extended-spectrum beta-lactamases (ESBL)-producing carbapenem-resistant Enterobacteriaceae (CRE-EB) (World Health Organization (WHO). Global priority list of antibiotic-resistant bacteria to guide research, discovery, and development of new antibiotics. 2017).

Antimicrobial peptides (AMPs) are considered to be promising candidates for the treatment of multidrug-resistant (MDR) bacteria. The basic design elements of AMPs include a hydrophobic nature, and a region of charged residues (generally cationic residues to enable interaction with bacterial cell surfaces) to disrupt the bacteria's cell membrane (Ganewatta, M. S. et al., Polymer 2015, 63, A1-A29). However, the development of AMPs is often hampered by their poor pharmacokinetic properties, low stability in biological fluids, toxicity towards mammalian cells due to poor selectivity, and generally high minimum inhibitory concentrations (MICs) as compared to classical antibiotics. Nevertheless, natural complex AMPs such as cyclic lipopeptides (e.g. polymyxin) are used clinically against difficult-to-treat Gram-negative bacterial infections. However, their high cost and toxicity dramatically restrict their use as a last-resort alternative. One antimicrobial peptide, colistin, has seen increased use as a last resort antibiotic recently as it is believed to kill bacteria by virtue of its ability to disrupt membrane integrity (Velkov, T. et al., J. Med. Chem. 2010, 53, 1898-1916). However, colistin requires intravenous administration and is nephrotoxic (Javan, A. O. et al., Eur. J. Clin. Pharmacol. 2015, 71, 801-810).

Besides peptides, synthetic polymers are widely used as disinfectants due to their high antibacterial potency. Most of these polymers are synthesized via free radical polymerization (FRP), ring opening polymerization (ROP) and post-functionalization which often involve multiple steps, difficult purification, usage of organic solvents, and are hard to scale-up. These antibacterial polymers usually display a high degree of toxicity with a limited range of antimicrobial efficacies.

Therefore, there is a need to develop new AMP-like analogues with improved properties.

### SUMMARY OF INVENTION

Aspects and embodiments of the invention will now be described by reference to the following numbered clauses.

1. A polymer or oligomer or a pharmaceutically acceptable solvate thereof comprising a first repeating unit comprising an imidazolium group and a biodegradable chain connected to an adjacent repeating unit.

2. The polymer or oligomer according to Clause 1, wherein the only repeating unit is the first repeating unit.

3. The polymer or oligomer according to Clause 1, wherein the polymer or oligomer further comprises a second repeating unit comprising an imidazolium group and a non-biodegradable alkyl chain or a further biodegradable alkyl chain connected to an adjacent repeating unit, optionally wherein the polymer or oligomer further comprises a second repeating unit comprising an imidazolium group and a non-biodegradable alkyl chain connected to an adjacent repeating unit.

4. The polymer or oligomer according to Clause 3, wherein one or more of the following apply:

(a) the polymer or oligomer comprises from 1 to 75 mol %, such as from 5 to 60 mol %, such as from 10 to 50 mol %, such as from 20 to 30 mol % of the first repeating unit; and

(b) the repeating units of the polymer or oligomer are randomly distributed or the repeating units may be formed as blocks, optionally wherein the repeating units of the polymer or oligomer are randomly distributed.

5. The polymer or oligomer according to any one of the preceding clauses, wherein the biodegradable chain in the first repeating unit comprises one or more biodegradable functional groups, where the one or more biodegradable functional groups are selected from one or more of the group consisting of urea, carbamate, acetal, amide, ester, carbonate ester, urethane, disulfide, anhydride, and hydrazone, optionally wherein:

(ai) the one or more biodegradable functional groups are selected from one or more of the group consisting of amide, ester, carbonate ester, urethane, disulfide, anhydride, and hydrazone;

(aii) the one or more biodegradable functional groups are selected from one or more of the group consisting of carbamate or, more particularly, amide, ester and carbonate ester; or

(aiii) the one or more biodegradable functional groups are amide.

6. The polymer or oligomer according to any one of the preceding clauses, wherein the number average molecular weight is from 800 to 10,000 Daltons, such as from 900 to 5,000 Daltons such as from 1,000 to 3,000 Daltons, such as from 1,000 to 2,000 Daltons.

7. The polymer or oligomer according to any one of the preceding clauses, wherein the polymer or oligomer has the formula I:

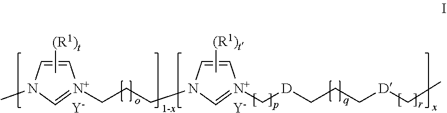

wherein:

x is from 0.01 to 1.0;

Y⁻ is a counterion;

o is from 0 to 10 (e.g. 0 to 6, such as from 1 to 5);

p is from 1 to 12;

q is from 0 to 14 (e.g. from 0 to 6);

r is from 0 to 12;

D is a biodegradable functional group;

D′ is a biodegradable functional group or a bond;

each R¹ is a branched or unbranched C₁₋₃ alkyl or a derivative thereof;

each t is 0, 1 or 2 (e.g. t is 0 or 1);

each t′ is 0, 1 or 2 (e.g. t′ is 0 or 1);

each R² is a branched or unbranched C₁₋₃ alkyl or a derivative thereof;

or a pharmaceutically acceptable solvate thereof.

8. The polymer or oligomer according to Clause 7, wherein one or more of the following apply:

(bi) each D is selected from urea, carbamate, acetal, amide, ester, carbonate ester, urethane, disulfide, anhydride, and hydrazone, optionally wherein:
(aa) each D is selected from one or more of the group consisting of amide, ester, carbonate ester, urethane, disulfide, anhydride, and hydrazone;
(ab) each D is selected from one or more of the group consisting of carbamate or, more particularly, amide, ester and carbonate ester; or
(ac) each D is selected from one or more of the group consisting of carbonate ester and amide (e.g. each D is an amide);

(bii) each D′ is selected from a bond, urea, carbamate, acetal, amide, ester, carbonate ester, urethane, disulfide, anhydride, and hydrazone, optionally wherein:
(ad) each D′ is selected from one or more of the group consisting of a bond, amide, ester, carbonate ester, urethane, disulfide, anhydride, and hydrazone;
(ae) each D′ is selected from one or more of the group consisting of a bond, amide, ester, carbamate and carbonate ester;
(af) each D′ is selected from one or more of the group consisting of a bond and amide;
(ag) each D′ is selected from one or more of the group consisting of amide, ester, carbonate ester, urethane, disulfide, anhydride, and hydrazone;
(ah) each D′ is selected from one or more of the group consisting of amide, ester, carbamate and carbonate ester;
(ai) each D′ is an amide;

(biii) Y⁻ is selected from one or more of the group consisting of halo, acetate, phosphate, sulfonate, and bis((trisfluoromethyl)sulfonyl)imide (N(Tf)₂⁻), optionally wherein Y⁻ is selected from one or more of the group consisting of chloro, acetate, phosphate, sulfonate, and bis((trisfluoromethyl)sulfonyl)imide (N(Tf)₂⁻);

(biv) x is from 0.01 to 1.0, such as from 0.025 to 0.75, such as from 0.05 to 0.6, such as from 0.1 to 0.5, such as from 0.2 to 0.3;

(bv) t and t′ are 0;

(bvi) p is from 1 to 6; and

(bvii) r is from 1 to 6.

9. The polymer or oligomer according to any one of the preceding clauses, wherein the polymer is selected from the group consisting of:

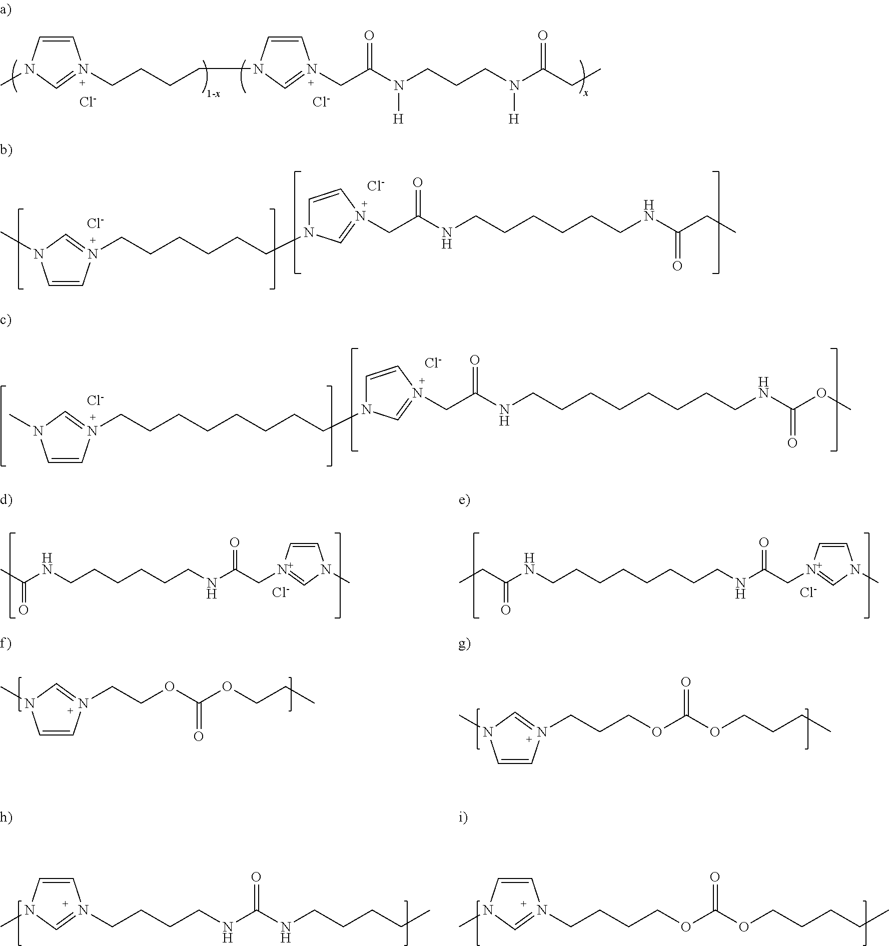

10. A molecule or a pharmaceutically acceptable solvate thereof comprising:

a first block of oligomeric repeating units, where each repeating unit comprises an imidazolium group and a non-biodegradable alkyl chain connected to an adjacent repeating unit;

a second block of oligomeric repeating units, where each repeating unit comprises an imidazolium group and a non-biodegradable alkyl chain connected to an adjacent repeating unit; and

a linking group connecting the first block and the second block together, wherein the linking group comprises one or more biodegradable functional groups.

11. The molecule according to Clause 10, wherein the one or more biodegradable functional groups are selected from one or more of the group consisting of urea, carbamate, acetal, amide, ester, carbonate ester, urethane, disulfide, anhydride, and hydrazone, optionally wherein:

(ci) the one or more biodegradable functional groups are selected from one or more of the group consisting of amide, ester, carbonate ester, urethane, disulfide, anhydride, and hydrazone;

(cii) the one or more biodegradable functional groups are selected from one or more of the group consisting of carbamate or, more particularly, amide, ester and carbonate ester;

(ciii) the one or more biodegradable functional groups are selected from one or both of amide and carbonate ester; or

(civ) the one or more biodegradable functional groups are amide.

12. The molecule according to Clause 10 or Clause 11 wherein, the molecular weight is from 1,000 Daltons to 5,000 Daltons, optionally wherein the molecular weight is from 1,000 Daltons to 4,000 Daltons.

13. The molecule according to any one of Clauses 10 to 12, wherein the molecule has the formula II:

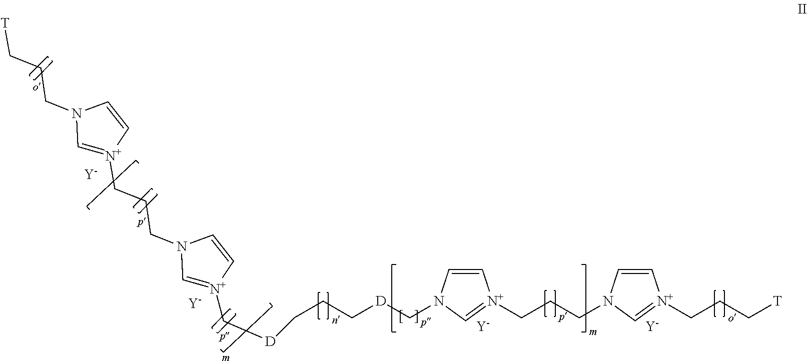

wherein:

each m is independently from 1 to 8 (e.g. from 1 to 6);

each Y⁻ is a counterion;

n′ is from 0 to 12;

each o′ is independently selected from 0 to 20;

each p′ is independently selected from 0 to 12 (e.g. from 0 to 6);

each p″ is independently selected from 0 to 12 (e.g. from 0 to 6);

each T is independently a terminal functional group selected from amine, ammonium, guanidinium, bisguanidinium, alkyl, and aryl;

each D is a biodegradable functional group, or a pharmaceutically acceptable solvate thereof.

14. The molecule according to Clause 13, wherein one or more of the following apply:

(di) each D is independently selected from urea, carbamate, acetal, amide, ester, carbonate ester, urethane, disulfide, anhydride, and hydrazone, optionally wherein:
(ba) each D is independently selected from one or more of the group consisting of amide, ester, carbonate ester, urethane, disulfide, anhydride, and hydrazone;
(bb) each D is independently selected from one or more of the group consisting of carbamate or, more particularly, amide, ester and carbonate ester; or
(bc) each D is amide;

(dii) Y⁻ is selected from one or more of the group consisting of halo, acetate, phosphate, sulfonate, and bis((trisfluoromethyl)sulfonyl)imide (N(Tf)₂⁻), optionally wherein Y⁻ is selected from one or more of the group consisting of chloro, acetate, phosphate, sulfonate, and bis((trisfluoromethyl)sulfonyl)imide (N(Tf)₂⁻); and

(dii) p″ is 0 to 6 (e.g. p″ is 0).

15. The molecule according to any one of Clauses 10 to 14, wherein the molecule is selected from the group consisting of:

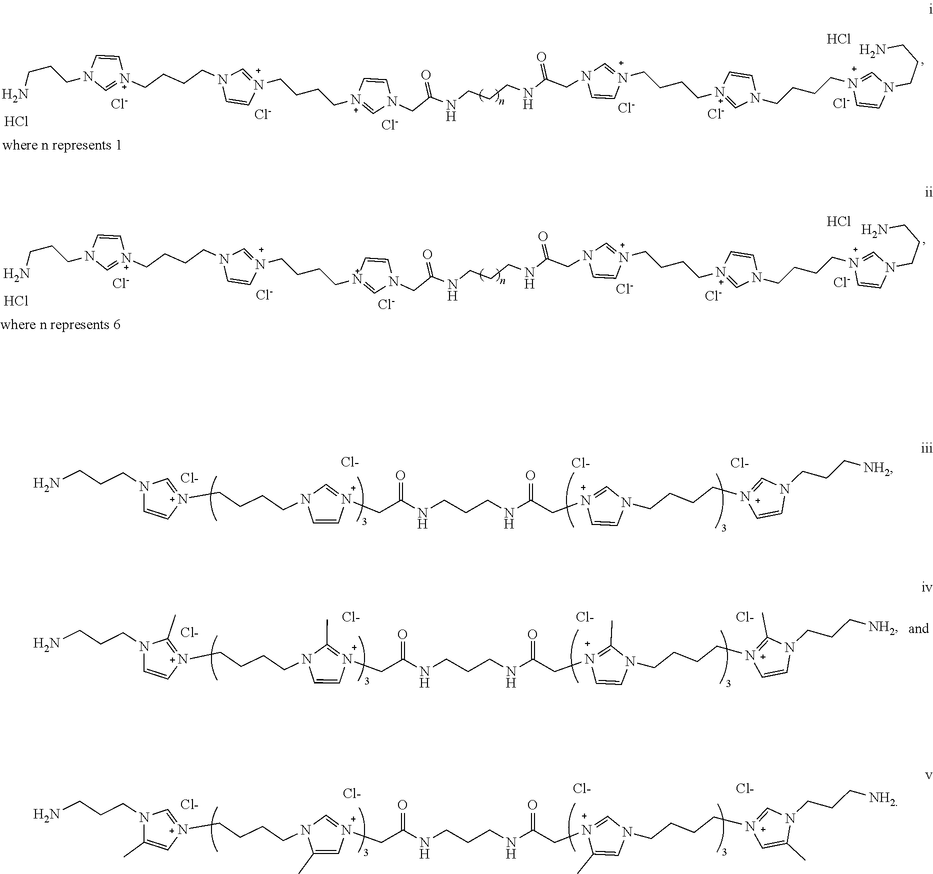

16. A polymer or oligomer or a pharmaceutically acceptable solvate thereof according to any one of Clauses 1 to 9 and/or a molecule or a pharmaceutically acceptable solvate thereof according to any one of Clauses 10 to 15 for use in medicine.

17. Use of a polymer or oligomer or a pharmaceutically acceptable solvate thereof according to any one of Clauses 1 to 9 and/or a molecule or a pharmaceutically acceptable solvate thereof according to any one of Clauses 10 to 15 in the manufacture of a medicament to treat a disease comprising a microbial infection.

18. A polymer or oligomer or a pharmaceutically acceptable solvate thereof according to any one of Clauses 1 to 9 and/or a molecule or a pharmaceutically acceptable solvate thereof according to any one of Clauses 10 to 15 for use in treating a disease comprising a microbial infection.

19. A method of treatment of a disease comprising a microbial infection comprising the step of administering to a subject in need thereof a therapeutically effective amount of a polymer or oligomer or a pharmaceutically acceptable solvate thereof according to any one of Clauses 1 to 9 and/or a therapeutically effective amount of a molecule or a pharmaceutically acceptable solvate thereof according to any one of Clauses 10 to 15.

20. The use of a polymer or oligomer or molecule according to Clause 17, the polymer or oligomer or molecule for use according to Clause 18 and the method according to Clause 19, wherein the microbial infection is an infected wound or cystic fibrosis.

21. An antiseptic formulation comprising a polymer or oligomer or a pharmaceutically acceptable solvate thereof according to any one of Clauses 1 to 9 and/or a molecule or a pharmaceutically acceptable solvate thereof according to any one of Clauses 10 to 15.

22. An article having a surface, wherein the surface is coated with a polymer or oligomer or a pharmaceutically acceptable solvate thereof according to any one of Clauses 1 to 9 and/or a molecule or a pharmaceutically acceptable solvate thereof according to any one of Clauses 10 to 15 to provide said surface of the article with antimicrobial properties, optionally wherein the article is a urinary catheter.

### DRAWINGS

FIG. 1 Chemical structures of polyimidazoliums (PIMs) synthesized and used in the experiments. The number of repeating subunits for each PIM was estimated by gel-permeation chromatography (GPC).

FIG. 2 shows the viability of (A) P. aeruginosa PAO1; and (B) MRSA LAC* treated with PIM1 (0.5-4 times the MIC for each bacterial species) in comparison to the control group with no PIM1 added. Cells were incubated at 37° C. in MHB and sampled at times indicated. Cell numbers were determined as colony-forming units (CFU) per mL by plate counting.

FIG. 3 depicts the propidium iodide (PI) staining of P. aeruginosa PAO1 cells. Fluorescence microscope images of (A) control cells (no antibiotic); (B) cells treated with colistin (1 times the MIC); (C) cells treated with PIM1 (1 times the MIC); and (D) Percent of propidium iodide (Pp-positive cells exposed to PIM1 (blue, left bars) or colistin (orange, right bars) at the concentrations indicated as determined by flow cytometry. Cells were incubated for 1 h in the presence of the antibiotic indicated prior to either microscope examination or flow cytometry.

FIG. 4 depicts the relative level of cell membrane electric potential (Δψ) of P. aeruginosa PAO1 cells exposed to increasing concentrations of PIM1, the ionophore gramicidin or the antibiotic gentamicin. Relative membrane potential was assessed by using the Δψ-sensitive fluorescent membrane probe DiS-C3-(5). An increase in DiS-C3-(5) fluorescence corresponds to a dissipation of Δψ. The ionophore gramicidin is a control agent known to collapse Δψ and the antibiotic gentamicin requires Δψ for uptake but does not dissipate Δψ. The shown relative dye fluorescence values 30 min after addition of test compound was the average of four tests (from 2 runs each with a duplicate) with (small) standard deviations.

FIG. 5 depicts the uptake of PIM1-FTIC conjugate by P. aeruginosa PAO1 and relationship between PIM1 activity and membrane potential. (A) Fluorescence microscope image of control cells (without PIM1) stained with membrane dye FMTM 4-64FX; (B) Fluorescence microscope image of cells treated with PIM1-FITC (1 times the MIC) and stained with FMTM 4-64FX; (C) MIC₉₀ (μg/mL) of PIM1 against P. aeruginosa in MHB with varied pH adjusted; and (D) MIC₉₀ (μg/mL) of PIM1 against P. aeruginosa PAO1 in the presence of valinomycin (left bars) or nigericin (right bars).

FIG. 6 shows the influence of metabolic status on P. aeruginosa PAO1 killing by PIM1. (A) Survival of stationary phase (Sta) bacteria and logarithmic phase (Log) bacteria after a 4-h exposure to PIM1, CST, or GEN; (B) Influence of fumarate (15 mM) on survival of stationary-phase bacteria. The same results for Sta-PIM1, Sta-CST, and Sta-GEN were used in A and B.

FIG. 7 shows the evolution of antibiotic resistance in (A) P. aeruginosa PAO1; and (B) MRSA LAC*. P. aeruginosa was grown in MHB and MRSA in TSB containing different concentrations of either PIM1 or ciprofloxacin. Bacteria showing visible growth at the highest concentration of antibiotic were transferred daily. Data are reported as the highest antibiotic concentration at which growth was observed and given as the fold increase in concentration relative to the MIC₉₀ on day 1.

FIG. 8 shows PIM1 treatment of a skin wound infection. Wounds were infected with the pan-antibiotic-resistant P. aeruginosa PAER and treated with 5 mg/kg imipenem (P. aeruginosa PAER is imipenem-resistant), or 0.1, 1, 5 or 10 mg/kg PIM1 4 h after infection. Bacterial numbers were determined by plate counting, and data for each individual mouse was reported. The horizontal lines indicate mean values and the bars±SD. *P<0.05, **P<0.01, and ns indicates P>0.05.

FIG. 9 shows that PIM1, but not PIM1D, has apparent toxicity. (A) Weight of mice treated with either a single 6 mg per kg dose of PIM1 (day 0) or daily doses of 15 mg per kg PIM1D for one week (days 0-6) via intraperitoneal (IP) injections. There were five mice in each group.

(B) alanine aminotransferase (ALT); (C) aspartate amino transferase (AST); and (D) blood nitrogen urea (BUN) levels in blood from mice treated with 15 mg per kg PIM1D daily for 7 days. Blood for mice given mock injections of saline solution was drawn just prior to initial injections and 1 day later. Blood for PIM1D-treated mice was drawn at 1, 3 and 7 days after administration of the first injection. There were five mice in each group and data for individual mice are shown as well as means and standard deviations.

FIG. 10 shows a schematic illustration of amide-incorporated degradable PIM1D synthesis: (A) Synthetic scheme of the degradable diamine A; and (B) Synthetic scheme of the amide-incorporated degradable PIM1D (n is the actual number-average degree of polymerization, and x is the mole fraction of the degradable repeat unit. n is about 10 and x is 20-30%).

FIG. 11 shows that PIM1D is effective in IP sepsis model induced by P. aeruginosa PAO1, MDR P. aeruginosa (PAER), MDR A. baumannii and methicillin-resistant S. aureus MRSA USA300. Colony forming unit (CFU) counting of liver in septicaemia model induced by (A) P. aeruginosa PAO1; (B) MDR P. aeruginosa (PAER); (C) MDR A. baumannii (AB-1); and (D) methicillin-resistant S. aureus MRSA USA300. Kaplan-Meier curve representing mice survival rate in septicaemia model induced by (E) PAO1; (F) PAER; (G) AB-1; and (H) MRSA USA300. Geomean±s.d., n=5. One-way ANOVA; ns, not significant, * P<0.05, ** P≤0.01, *** P≤0.001.

FIG. 12 shows CFUs counting of kidney, spleen and IP fluid in septicaemia model induced by (A-C) P. aeruginosa PAO1; (D-F) MDR P. aeruginosa (PAER); (G-I) MDR A. baumannii (AB-1); and (J-L) methicillin-resistant S. aureus MRSA USA300. CFU counting of (A) kidney; (B) spleen; and (C) IP fluid in septicaemia model induced by PAO1. CFU counting of (D) kidney; (E) spleen; and (F) IP fluid in septicaemia model induced by PAER. CFU counting of (G) kidney; (H) spleen; and (I) IP fluid in septicaemia model induced by AB-1. CFU counting of (J) kidney; (K) spleen; and (L) IP fluid in septicaemia model induced by MRSA USA300. * P<0.05, ** P≤1.01, and ns is not significant (two-tailed Student's t-test).

FIG. 13 depicts blood biochemistry analysis at day 1, day 3 and day 7 in which mice had received one dosing, three consecutive dosings and seven consecutive dosings, respectively, of PIM1D (15 mg/kg) through IP injection. (A) Alanine Aminotransferase (ALT); (B) Aspartate

Aminotransferase (AST); (C) Blood urea nitrogen (BUN); (D) Creatinine (CRE); (E) Total bilirubin (TBIL); (F) total protein (TP); (G) globulin (GLO); and (H) glucose (GLU). Blood biochemical parameters from each mouse are shown as individual points with error bars representing the deviation of each experimental group.

FIG. 14 shows that PIM1D is effective in neutropenic lung model using methicillin-resistant S. aureus MRSA USA300 and K. pneumoniae ATCC 13883. CFU counting of Lung in neutropenic lung model induced by (A) MRSA USA300; and (B) K. pneumoniae. Kaplan-Meier curve representing mice survival rate in neutropenic lung model induced by (C) MRSA USA300; and (D) K. pneumoniae. Geomean±s.d. One-way ANOVA, ns, not significant, * P<0.05, ** P<0.01.

FIG. 15 depicts the synthesis of PIM1 bromide monomer.

FIG. 16 depicts the synthesis of PIM1-Br.

FIG. 17 depicts the general synthesis of nondegradable main-chain cationic PIMs.

FIG. 18 depicts the synthesis of the TFA salt of diamide diamine (n=4, 6, 8, 10 and 12) monomer.

FIG. 19 depicts the general synthesis of degradable main-chain cationic PIMs by (a) copolymerization of degradable and nondegradable diamines; and (b) homopolymerization of degradable diamine.

FIG. 20 depicts the chemical structures of a series of PIMs (P1-P6).

FIG. 21 depicts the antibiofilm properties of PIMs and benzalkonium chloride (BAC, reference) measured by minimum biofilm eradication concentration (MBEC) assay. Viable bacterial counts of MRSA BAA39 on each microtiter plate peg after 4 h of treatments with PIMs or BAC.

FIG. 22 depicts the antibiofilm properties of PIMs and BAC (reference) measured by MBEC assay. Viable bacterial counts of PAO1 on each microtiter plate peg after 4 h of treatments with PIMs or BAC.

FIG. 23 depicts the synthesis of 2+2 carbonate monomer.

FIG. 24 depicts the synthetic scheme of (a) carbonate monomer; and (b) carbonate-incorporated biodegradable PIM D2.

FIG. 25 depicts the synthetic scheme of OIM1D-3C-6 and OIM1D-3C-8.

FIG. 26 depicts the efficacy of OIM1D-30-8 in neutropenic lung infection model induced by (A) multidrug-resistant K. pneumonia; (B) methicillin-resistant S. aureus; (C) mice weight change after addition of 20 mg/kg compounds via intranasal delivery; and (D) efficacy of OIM1D-3C-8/OIM1D-3C-6 (2:1 wt. % and 1:1 wt. %) in neutropenic lung infection model induced by multidrug resistant K. pneumonia.

FIG. 27 depicts a schematic illustration of varied diamines with degradable linker for biodegradable PIMs synthesis (p=1-12, q=0-10).

### DESCRIPTION

Disclosed herein are a series of poly(alkylated imidazolium) (PIM) salts, which contain one or more degradable moieties in the alkyl chains. Surprisingly, these PIM salts exhibit excellent broad-spectrum antimicrobial properties against a range of clinically important ESKAPE (Enterococcus faecium, Staphylococcus aureus, Klebsiella pneumoniae, Acinetobacter baumannii, Pseudomonas aeruginosa, and Enterobacter) bacteria species, while having low toxicity to mammalian cells. Indeed, PIMs with a higher molar fraction of degradable linker moieties displayed higher biocompatibility. In addition, the polymers disclosed herein have been found to be active against both Gram-positive and Gram-negative bacteria.

The term “Gram-positive bacteria” refers to bacteria having cell walls with high amounts of peptidoglycan. Gram-positive bacteria are identified by their tendency to retain crystal violet and stain dark blue or violet in the Gram staining protocol.

The term “Gram-negative bacteria” refers to bacteria having thinner peptidoglycan layers which do not retain the crystal violet stain in the Gram staining protocol and instead retain the counterstain, typically safranin. Gram-negative bacteria stain red or pink in the Gram staining protocol.

Thus, in a first aspect of the invention, there is disclosed a polymer or oligomer or a pharmaceutically acceptable solvate thereof comprising a first repeating unit comprising an imidazolium group and a biodegradable chain connected to an adjacent repeating unit.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “a first repeating unit” includes a plurality of said repeating units and does not exclude the possibility of further (different) repeating units also being present, and the like.

When used herein, the term “biodegradeable chain” refers to a linking group that connects one imidazolium group to another. This biodegradeable chain may comprise one or more biodegradable functional groups.

Any suitable biodegradable functional group may be used herein. When used herein, the term biodegradable functional group is intended to refer to a functional group that can be cleaved in the environment and/or in vivo either by chemical or biological materials present in the ambient environment in which an oligomer, polymer or molecule of the current invention may find itself in. Non-limiting examples of biodegradable functional groups that may be mentioned herein include urea, carbamate, acetal, amide, ester, carbonate ester, urethane, disulfide, anhydride, and hydrazone. Said functional groups may be susceptible to cleavage by chemicals or biological materials in the ambient environment (e.g. esters may be cleaved due to acidic or basic conditions of the environment, or due to the presence of enzymes). This cleavage may take place in vivo or ex vivo, depending on the way that the materials disclosed herein are used and/or disposed of. Examples of functional groups that may not be biodegradable include ether linkages.

In embodiments mentioned of the polymer or oligomer of the first aspect of the invention, the only repeating unit may be the first repeating unit. However, in alternative embodiments of the first aspect of the invention, the polymer or oligomer may further comprise a second repeating unit comprising an imidazolium group and a non-biodegradable alkyl chain or a further biodegradable alkyl chain connected to an adjacent repeating unit. In certain embodiments, where the polymer or oligomer may further comprise a second repeating unit, one or more of the following may apply:

(a) the polymer or oligomer may comprise from 1 to 75 mol %, such as from 5 to 60 mol %, such as from 10 to 50 mol %, such as from 20 to 30 mol % of the first repeating unit; and

(b) the repeating units of the polymer or oligomer may be randomly distributed or the repeating units may be formed as blocks, more particularly the repeating units of the polymer or oligomer may be randomly distributed. In particular embodiments of the above, the second repeating unit may be a second repeating unit comprising an imidazolium group and a non-biodegradable alkyl chain.

In embodiments of the first aspect of the invention that may be mentioned herein, the biodegradable chain in the first repeating unit comprises one or more biodegradable functional groups, where the one or more biodegradable functional groups are selected from one or more of the group consisting of urea, carbamate, acetal, amide, ester, carbonate ester, urethane, disulfide, anhydride, and hydrazone, optionally wherein:

(ai) the one or more biodegradable functional groups may be selected from one or more of the group consisting of amide, ester, carbonate ester, urethane, disulfide, anhydride, and hydrazone;

(aii) the one or more biodegradable functional groups may be selected from one or more of the group consisting of carbamate or, more particularly, amide, ester and carbonate ester; or

(aiii) the one or more biodegradable functional groups may be amide.

In embodiments of the first aspect of the invention that may be mentioned herein, the number average molecular weight may be from 800 to 10,000 Daltons, such as from 900 to 5,000 Daltons, such as from 1,000 to 3,000 Daltons, such as from 1,000 to 2,000 Daltons.

In particular embodiments of the first aspect of the invention that may be mentioned herein, the polymer or oligomer may have the formula I:

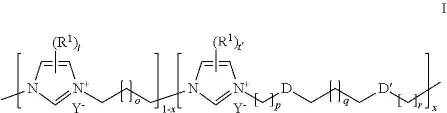

wherein:

x is from 0.01 to 1.0;

Y⁻ is a counterion;

o is from 0 to 10 (e.g. 0 to 6, such as from 1 to 5);

p is from 1 to 12;

q is from 0 to 14 (e.g. from 0 to 6);

r is from 0 to 12;

D is a biodegradable functional group;

D′ is a biodegradable functional group or a bond;

each R¹ is a branched or unbranched C₁₋₃ alkyl or a derivative thereof;

each t is 0, 1 or 2;

each t′ is 0, 1 or 2;

each R² is a branched or unbranched C₁₋₃ alkyl or a derivative thereof;

or a pharmaceutically acceptable solvate thereof.

When used herein, the term “C₁₋₃ alkyl” may refer to, for example, ethyl, propyl, (e.g. n-propyl or isopropyl), or more preferably, methyl. Derivatives of C₁₋₃ alkyl may refer to substituted C₁₋₃ alkyl groups. Examples of substituted C₁₋₃ alkyl groups that may be mentioned herein include, but are not limited to halo (e.g. Br, Cl or, more particularly F). A particular derivative that may be mentioned herein is CF₃.

In embodiments of the invention relating to the polymer or oligomer according to formula I, one or more of the following apply:

(bi) each D may be selected from urea, carbamate, acetal, amide, ester, carbonate ester, urethane, disulfide, anhydride, and hydrazone, optionally wherein:
(aa) each D may be selected from one or more of the group consisting of amide, ester, carbonate ester, urethane, disulfide, anhydride, and hydrazone;
(ab) each D may be selected from one or more of the group consisting of carbamate or, more particularly, amide, ester and carbonate ester; or
(ac) each D may be selected from one or more of the group consisting of carbonate ester and amide (e.g. each D is an amide);

(bii) each D′ may be selected from a bond, urea, carbamate, acetal, amide, ester, carbonate ester, urethane, disulfide, anhydride, and hydrazone, optionally wherein:
(ad) each D′ may be selected from one or more of the group consisting of a bond, amide, ester, carbonate ester, urethane, disulfide, anhydride, and hydrazone;
(ae) each D′ may be selected from one or more of the group consisting of a bond, amide, ester, carbamate and carbonate ester;
(af) each D′ may be selected from one or more of the group consisting of a bond and amide;
(ag) each D′ may be selected from one or more of the group consisting of amide, ester, carbonate ester, urethane, disulfide, anhydride, and hydrazone;
(ah) each D′ may be selected from one or more of the group consisting of amide, ester, carbamate and carbonate ester;
(ai) each D′ may be an amide;

(biii) Y⁻ may be selected from one or more of the group consisting of halo, acetate, phosphate, sulfonate, and bis((trisfluoromethyl)sulfonyl)imide (N(Tf)₂⁻), optionally wherein Y⁻ may be selected from one or more of the group consisting of chloro, acetate, phosphate, sulfonate, and bis((trisfluoromethyl)sulfonyl)imide (N(Tf)₂⁻);

(biv) x may be from 0.01 to 1.0, such as from 0.025 to 0.75, such as from 0.05 to 0.6, such as from 0.1 to 0.5, such as from 0.2 to 0.3;

(bv) t and t′ may be 0;

(bvi) p may be from 1 to 6; and

(bvii) r may be from 1 to 6.

As will be appreciated, any combination of the variables described above is envisaged.

It will be appreciated that D and D′ may be the same or different. In particular embodiments of the invention, D′ may be a biodegradable functional group, such that the biodegradable chain has two biodegradable functional groups. However, in other embodiments (e.g. when D is carbamate), D′ may be a bond.

Embodiments of the invention that may be mentioned include those in which the polymer or oligomer of the first aspect of the invention (such as a polymer or oligomer of formula I) is a compound selected from the list:

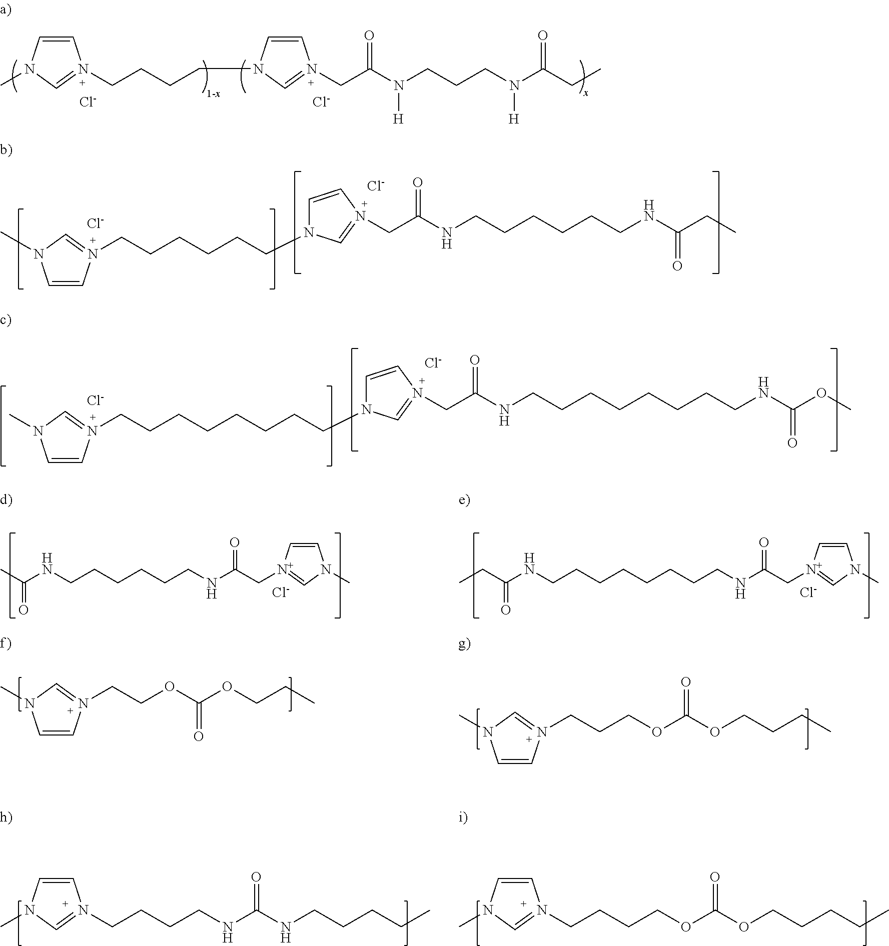

When the polymer or oligomer contains two repeating units, the amount of the repeating unit that contains the one or more biodegradable functional groups may be from 1 to 99 mol %, such as from 5 to 95 mol %, such as from 10 to 90 mol %, such as from 20 to 80 mol %, such as from 25 to 75 mol %, such as 50 mol %. In specific embodiments that may be mentioned herein, the amount of the repeating unit that contains the one or more biodegradable functional groups may be from 20 to 30 mol %.

For the avoidance of doubt, it is explicitly contemplated that where a number of numerical ranges related to the same feature are cited herein, the end points for each range are intended to be combined in any order to provide further contemplated (and implicitly disclosed) ranges.

Thus, for the ranges listed above (and for the first repeating unit in general), the following ranges are contemplated:

from 1 to 5 mol %, from 1 to 10 mol %, from 1 to 20 mol %, from 1 to 25 mol %, from 1 to 30 mol %, from 1 to 50 mol %, from 1 to 60 mol %, from 1 to 75 mol %, from 1 to 80 mol %, from 1 to 95 mol %, from 1 to 99 mol %;

from 5 to 10 mol %, from 5 to 20 mol %, from 5 to 25 mol %, from 5 to 30 mol %, from 5 to 50 mol %, from 5 to 60 mol %, from 5 to 75 mol %, from 5 to 80 mol %, from 5 to 95 mol %, from 5 to 99 mol %;

from 10 to 20 mol %, from 10 to 25 mol %, from 10 to 30 mol %, from 10 to 50 mol %, from 10 to 60 mol %, from 10 to 75 mol %, from 10 to 80 mol %, from 10 to 95 mol %, from 10 to 99 mol %;

from 20 to 25 mol %, from 20 to 30 mol %, from 20 to 50 mol %, from 20 to 60 mol %, from 20 to 75 mol %, from 20 to 80 mol %, from 20 to 95 mol %, from 20 to 99 mol %;

from 25 to 30 mol %, from 25 to 50 mol %, from 25 to 60 mol %, from 25 to 75 mol %, from 25 to 80 mol %, from 25 to 95 mol %, from 25 to 99 mol %;

from 30 to 50 mol %, from 30 to 60 mol %, from 30 to 75 mol %, from 30 to 80 mol %, from 30 to 95 mol %, from 30 to 99 mol %;

from 50 to 60 mol %, from 50 to 75 mol %, from 50 to 80 mol %, from 50 to 95 mol %, from 50 to 99 mol %;

from 60 to 75 mol %, from 60 to 80 mol %, from 60 to 95 mol %, from 60 to 99 mol %;

from 75 to 80 mol %, from 75 to 95 mol %, from 75 to 99 mol %;

from 80 to 95 mol %, from 80 to 99 mol %; and

from 95 to 99 mol %.

In particular embodiments of (b) and (c) in the table above, the repeating unit that contains the one or more biodegradable functional groups may be present in an amount of 50 mol %.

In embodiments of the invention that may be mentioned herein, the polymers and oligomers in the table above may have a number average molecular weight of from 960 to 3,000 Daltons, such as from 966 to 2,800 Daltons.

In a second aspect of the invention, there is disclosed a molecule or a pharmaceutically acceptable solvate thereof comprising:
a first block of oligomeric repeating units, where each repeating unit comprises an imidazolium group and a non-biodegradable alkyl chain connected to an adjacent repeating unit;
a second block of oligomeric repeating units, where each repeating unit comprises an imidazolium group and a non-biodegradable alkyl chain connected to an adjacent repeating unit; and
a linking group connecting the first block and the second block together, wherein the linking group comprises one or more biodegradable functional groups.

In embodiments of the second aspect of the invention that may be mentioned herein, the one or more biodegradable functional groups may be selected from one or more of the group consisting of urea, carbamate, acetal, amide, ester, carbonate ester, urethane, disulfide, anhydride, and hydrazone, optionally wherein:

(ci) the one or more biodegradable functional groups may be selected from one or more of the group consisting of amide, ester, carbonate ester, urethane, disulfide, anhydride, and hydrazone;

(cii) the one or more biodegradable functional groups may be selected from one or more of the group consisting of carbamate or, more particularly, amide, ester and carbonate ester;

(ciii) the one or more biodegradable functional groups may be selected from one or both of amide and carbonate ester; or

(civ) the one or more biodegradable functional groups may be amide.

In embodiments of the second aspect of the invention, the molecular weight of the molecule may be from 1,000 Daltons to 5,000 Daltons, optionally wherein the molecular weight is from 1,000 Daltons to 4,000 Daltons.

In particular embodiments of the second aspect of the invention that may be mentioned herein, the molecule may have the formula II:

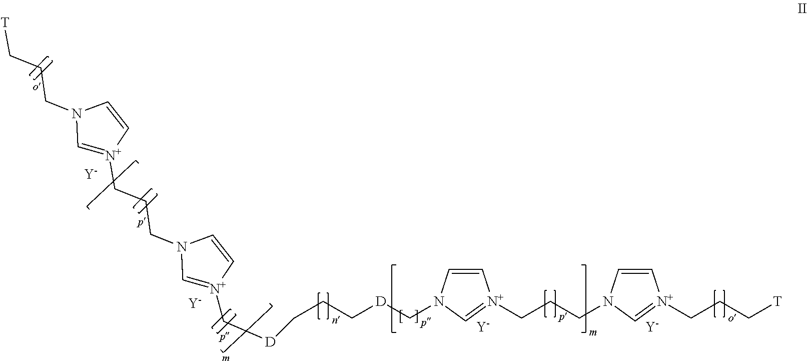

wherein:

each m is independently from 1 to 8 (e.g. from 1 to 6);

each Y⁻ is a counterion;

n′ is from 0 to 12;

each o′ is independently selected from 0 to 20;

each p′ is independently selected from 0 to 12 (e.g. from 0 to 6);

each p″ is independently selected from 0 to 12 (e.g. from 0 to 6);

each T is independently a terminal functional group selected from amine, ammonium, guanidinium, bisguanidinium, alkyl, and aryl;

each D is a biodegradable functional group, or a pharmaceutically acceptable solvate thereof.

In embodiments of the invention relating to the polymer or oligomer according to formula II, one or more of the following may apply:

(di) each D may be independently selected from urea, carbamate, acetal, amide, ester, carbonate ester, urethane, disulfide, anhydride, and hydrazone, optionally wherein:
(ba) each D may be independently selected from one or more of the group consisting of amide, ester, carbonate ester, urethane, disulfide, anhydride, and hydrazone;
(bb) each D may be independently selected from one or more of the group consisting of carbamate or, more particularly, amide, ester and carbonate ester; or
(bc) each D may be amide;

(dii) Y⁻ may be selected from one or more of the group consisting of halo, acetate, phosphate, sulfonate, and bis((trisfluoromethyl)sulfonyl)imide (N(Tf)₂⁻), optionally wherein Y⁻ is selected from one or more of the group consisting of chloro, acetate, phosphate, sulfonate, and bis((trisfluoromethyl)sulfonyl)imide (N(Tf)₂⁻); and

(dii) p″ may be 0 to 6 (e.g. p″ is 0).

Embodiments of the invention that may be mentioned include those in which the molecule of the second aspect of the invention is selected from the list:

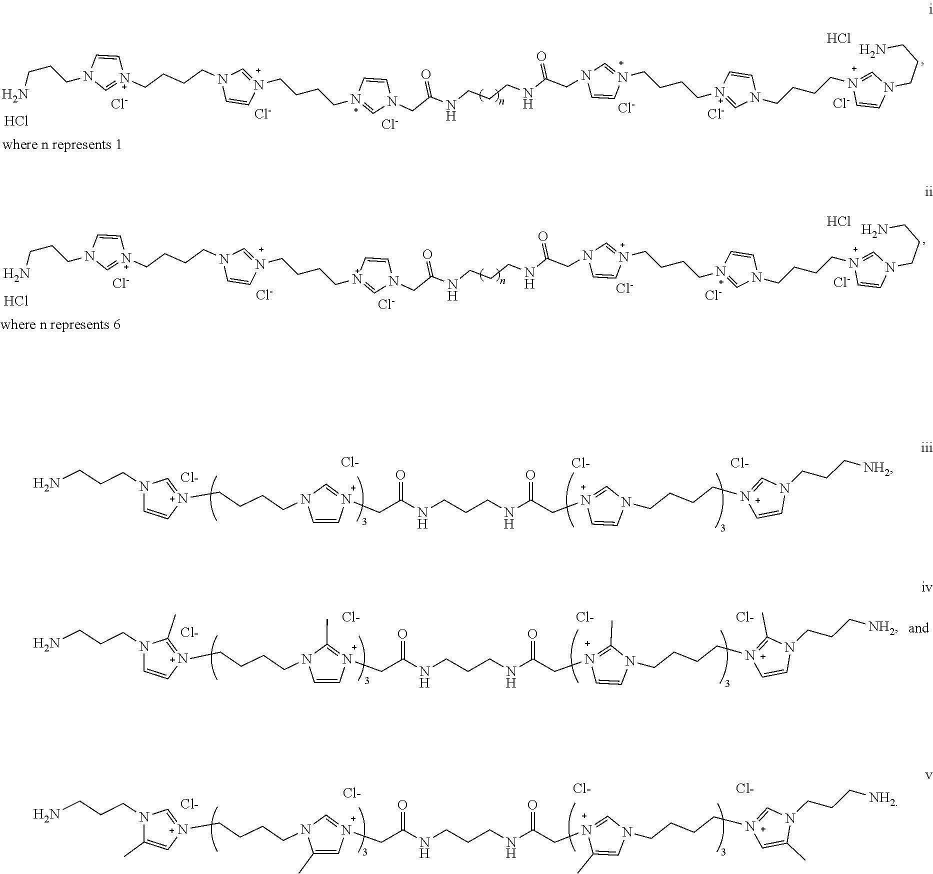

References herein (in any aspect or embodiment of the invention) to the polymers, oligomers and molecules herein (including the polymers or oligomers of formula I or the molecules of formula II) include references to such compounds per se, to tautomers of such compounds, as well as to pharmaceutically acceptable salts or solvates, or pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula I or formula II with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I or formula II in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g.(+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As will be appreciated, the polymers, oligomers and molecules described herein may already include a counterion, but this counterion may be swapped for a different one should that be desired. For example, the polymers, oligomers and molecules described herein may be subjected to an ion-exchange column so as to replace one counterion for a different one.

As mentioned above, also encompassed by polymers, oligomers and molecules described herein are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

“Pharmaceutically functional derivatives” of polymers, oligomers and molecules described herein as defined herein includes ester derivatives and/or derivatives that have, or provide for, the same biological function and/or activity as any relevant compound of the invention. Thus, for the purposes of this invention, the term also includes prodrugs of polymers, oligomers and molecules described herein.

The term “prodrug” of a relevant polymer, oligomer or molecule described herein includes any compound that, following oral or parenteral administration, is metabolised in vivo to form the active agent in an experimentally-detectable amount, and within a predetermined time (e.g. within a dosing interval of between 6 and 24 hours (i.e. once to four times daily)).

Prodrug polymers, oligomers and molecules described herein may be prepared by modifying functional groups present on the compound in such a way that the modifications are cleaved in vivo when such prodrug is administered to a mammalian subject. The modifications typically are achieved by synthesizing the parent compound with a prodrug substituent. Prodrugs include polymers, oligomers and molecules described herein wherein a hydroxyl, amino, sulfhydryl, carboxyl or carbonyl group in a compound of formula I or formula II is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. I-92, Elsevier, New York-Oxford (1985).

The polymers, oligomers and molecules described herein may contain double bonds and may thus exist as E (entgegen) and Z (zusammen) geometric isomers about each individual double bond. All such isomers and mixtures thereof are included within the scope of the invention.

The polymers, oligomers and molecules described herein may exist as regioisomers and may also exhibit tautomerism. All tautomeric forms and mixtures thereof are included within the scope of the invention.

The polymers, oligomers and molecules described herein may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound which confers a therapeutic effect on the treated patient (e.g. sufficient to treat or prevent the disease). The effect may be objective (i.e. measurable by some test or marker) or subjective (i.e. the subject gives an indication of or feels an effect).

The term “halo”, when used herein, includes references to fluoro, chloro, bromo and iodo.

Unless otherwise stated, the term “aryl” when used herein includes C₆₋₁₄ (such as C₆₋₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

Unless otherwise stated, the term “alkyl” refers to an unbranched or branched, acyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl.

Further embodiments of the invention that may be mentioned include those in which the polymers, oligomers and molecules described herein is isotopically labelled. However, other particular embodiments of the invention that may be mentioned include those in which the polymers, oligomers and molecules described herein is not isotopically labelled.

The term “isotopically labelled”, when used herein includes references to polymers, oligomers and molecules described herein in which there is a non-natural isotope (or a non-natural distribution of isotopes) at one or more positions in the compound. References herein to “one or more positions in the compound” will be understood by those skilled in the art to refer to one or more of the atoms of the polymers, oligomers and molecules described herein. Thus, the term “isotopically labelled” includes references to polymers, oligomers and molecules described herein that are isotopically enriched at one or more positions in the compound.

The isotopic labelling or enrichment of the polymers, oligomers and molecules described herein may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the polymers, oligomers and molecules described herein is labelled or enriched with a radioactive or nonradioactive isotope, polymers, oligomers and molecules described herein that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

The compounds disclosed herein may be particularly useful in the treatment of microbial infections. Thus, in a third aspect of the invention, there is provided a polymer or oligomer or a pharmaceutically acceptable solvate thereof according to the first aspect of the invention and any technically sensible combination of its embodiments and/or a molecule or a pharmaceutically acceptable solvate thereof according to the second aspect of the invention and any technically sensible combination of its embodiments for use in medicine.

Further, in a fourth aspect of the invention, there is provided:

(AAA) use of a polymer or oligomer or a pharmaceutically acceptable solvate thereof according to the first aspect of the invention and any technically sensible combination of its embodiments and/or a molecule or a pharmaceutically acceptable solvate thereof according to the second aspect of the invention and any technically sensible combination of its embodiments in the manufacture of a medicament to treat a disease comprising a microbial infection;

(AAB) a polymer or oligomer or a pharmaceutically acceptable solvate thereof according to the first aspect of the invention and any technically sensible combination of its embodiments and/or a molecule or a pharmaceutically acceptable solvate thereof according to the second aspect of the invention and any technically sensible combination of its embodiments for use in treating a disease comprising a microbial infection; and

(AAC) a method of treatment of a disease comprising a microbial infection comprising the step of administering to a subject in need thereof a therapeutically effective amount of a polymer or oligomer or a pharmaceutically acceptable solvate thereof according to the first aspect of the invention and any technically sensible combination of its embodiments and/or a therapeutically effective amount of a molecule or a pharmaceutically acceptable solvate thereof according to the second aspect of the invention and any technically sensible combination of its embodiments.

In an embodiment of the fourth aspect of the invention, the microbial infection may relate to an infected wound or cystic fibrosis.

The term “microbial infection” covers any disease or condition caused by a microbial organism in or on a subject. Examples of microbial infections include, but are not limited to, tuberculosis caused by mycobacteria, burn wound infections caused by pseudomonas etc., skin infections caused by S. aureus, wound infections caused by pseudomonas and A. baumannii, and Sepsis. The term “fungal infection” covers any disease or condition caused by a microbial organism in or on a subject. Examples of microbial infections include, but are not limited to, athlete's foot, ringworm, yeast infections, and jock itch.

A non-limiting list of bacteria that may be susceptible to the polymers and copolymers of the invention include: Acidothermus cellulyticus, Actinomyces odontolyticus, Alkaliphilus metalliredigens, Alkaliphilus oremlandii, Arthrobacter aurescens, Bacillus amyloliquefaciens, Bacillus clausii, Bacillus halodurans, Bacillus licheniformis, Bacillus pumilus, Bacillus subtilis, Bifidobacterium adolescentis, Bifidiobacterium longum, Caldicellulosiruptor saccharolyticus, Carboxydothermus hydrogenoformans, Clostridium acetobutylicum, Clostridium beijerinckii, Clostridium botulinum, Clostridium cellulolyticum, Clostridium difficile, Clostridium kluyveri, Clostridium leptum, Clostridium novyi, Clostridium perfringens, Clostridium tetani, Clostridium thermocellum, Corynebacterium diphtheriae, Corynebacterium efficiens, Corynebacterium glutamicum, Corynebacterium jeikeium, Corynebacterium urealyticum, Desulfitobacterium hafniense, Desulfotomaculum reducens, Eubacterium ventriosum, Exiguobacterium sibiricum, Fingoldia magna, Geobacillus kaustophilus, Geobacillus the rmodenitrificans, Janibacter sp., Kineococcus radiotolerans, Lactobacillus fermentum, Listeria monocytogenes, Listeria innocua, Listeria welshimeri, Moorella thermoacetica, Mycobacterium avium, Mycobacterium bovis, Mycobacterium gilvum, Mycobacterium leprae, Mycobacterium paratuberculosis, Mycobacterium smegmatis, Mycobacterium tuberculosis, Mycobacterium ulcerans, Mycobacterium vanbaalenii, Nocardioides sp., Nocardia farcinica, Oceanobacillus iheyensis, Pelotomaculum thermopropionicum, Rhodococcus sp., Saccharopolyspora erythraea, coagulase-negative Staphylococcus species, Staphylococcus aureus, methicillin resistant Staphylococcus aureus (MRSA), Staphylococcus epidermidis, methicillin resistant Staphylococcus epidermidis (MRSE), Streptococcus agalactiae, Streptococcus gordonii, Streptococcus mitis, Streptococcus oralis, Streptococcus pneumoniae, Streptococcus sanguinis, Streptococcus suis, Streptomyces avermitilis, Streptomyces coelicolor, Thermoanaerobacter ethanolicus, Thermoanaerobacter tengcongensis, and combinations thereof.

As noted above, the polymers, oligomers and molecules of the invention may be used in the treatment of microbial and fungal infections. Thus, there is also provided a pharmaceutical composition comprising a polymer, oligomer or molecule of the invention and a pharmaceutically acceptable carrier.

Polymers, oligomers or molecules of the invention may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

When used herein, reference to polymers and oligomers of the invention relates to the polymers and oligomers of the first aspect of the invention (and any technically sensible combination of its embodiments), while reference to molecules of the invention relates to the polymers and oligomers of the second aspect of the invention (and any technically sensible combination of its embodiments).

Polymers, oligomers or molecules of the invention will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of the polymer, oligomer or molecule of the invention in any pharmaceutical formulation used in accordance with the present invention will depend on various factors, such as the severity of the condition to be treated, the particular patient to be treated, as well as the compound(s) which is/are employed. In any event, the amount of polymer, oligomer or molecule of the invention in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, polymers, oligomers or molecules of the invention may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a polymer or copolymer of the invention.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The aspects of the invention described herein (e.g. the above-mentioned polymers, oligomers and molecules, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

Polymers, oligomers or molecules of the invention may be prepared in accordance with techniques that are well known to those skilled in the art, for example as described hereinafter in the examples section.

Polymers, oligomers or molecules of the invention may be isolated from their reaction mixtures using conventional techniques (e.g. recrystallisation, column chromatography, preparative HPLC, etc.).

In a fifth aspect of the invention, there is provided an antiseptic formulation comprising a polymer or oligomer or a pharmaceutically acceptable solvate thereof according to the first aspect of the invention and any technically sensible combination of its embodiments and/or a molecule or a pharmaceutically acceptable solvate thereof according to the second aspect of the invention and any technically sensible combination of its embodiments.

Given the above, the polymers, oligomers or molecules of the invention may be used as antimicrobial active ingredients in personal care preparations, for example antiseptics, shampoos, bath additives, hair-care products, liquid and solid soaps (based on synthetic surfactants and salts of saturated and/or unsaturated fatty acids), lotions and creams and other aqueous or alcoholic solutions, e.g. cleansing solutions for the skin. Thus, the antiseptic formulation referred to above may refer to any of the formulations listed in this paragraph.

When used as a simple antiseptic composition (i.e. directed to use as an antiseptic only), the antispectic formulation composition may comprise from 0.01 to 20% by weight, such as from 0.5 to 10% by weight of a polymer, oligomer or molecule of the invention. It will be appreciated that more than one polymer, oligomer or molecule of the invention may form part of the antiseptic composition.

The polymers, oligomers or molecules of the invention exhibit a pronounced antimicrobial action, especially against pathogenic gram-positive and gram-negative bacteria and so may also act against bacteria of skin flora, e.g. Corynebacterium xerosis (bacteria that cause body odour), and also against yeasts and moulds. They are therefore also suitable in the disinfection of the skin and mucosa and also of integumentary appendages (hair), and so may also be suitable in the disinfection of the hands and of wounds.

Thus, there is also provided an antimicrobial and/or antifungal detergent composition comprising a polymer, oligomer or molecule of the invention and a surfactant. It will be appreciated that the composition may also contain additional cosmetically tolerable carriers and/or adjuvants. Said composition may in particular be in the form of a shampoo or in the form of a solid or liquid soap, though other compositions as described hereinabove are also contemplated (e.g. other hair-care products, lotions and creams etc.).

The detergent composition may comprise from 0.01 to 15% by weight, such as from 0.5 to 10% by weight of a polymer or copolymer of the invention. It will be appreciated that more than one polymer and copolymer of the invention may form part of the detergent composition.

Depending upon the form of the detergent composition, it will comprise, in addition to the polymer or copolymer of the invention, further constituents, for example sequestering agents, colourings, perfume oils, thickening or solidifying (consistency regulator) agents, emollients, UV absorbers, skin-protective agents, antioxidants, additives that improve mechanical properties, such as dicarboxylic acids and/or Al, Zn, Ca and Mg salts of C₁₄-C₂₂ fatty acids, and optionally preservatives.

The detergent composition may be formulated as a water-in-oil or oil-in-water emulsion, as an alcoholic or alcohol-containing formulation, as a vesicular dispersion of an ionic or non-ionic amphiphilic lipid, as a gel, a solid stick or as an aerosol formulation.

As a water-in-oil or oil-in-water emulsion, the detergent composition may comprise from 5 to 50 wt % of an oily phase, from 5 to 20 wt % of an emulsifier and from 30 to 90 wt % water. The oily phase may contain any oil suitable for cosmetic formulations, e.g. one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Detergent compositions may be provided in a wide variety of preparations. Examples of suitable compositions include, but are not limited to skin-care preparations (e.g. skin-washing and cleansing preparations in the form of tablet-form or liquid soaps, soapless detergents or washing pastes), bath preparations, (e.g. liquid compositions such as foam baths, milks, shower preparations or solid bath preparations), shaving preparations (e.g. shaving soap, foaming shaving creams, non-foaming shaving creams, foams and gels, preshave preparations for dry shaving, aftershaves or after-shave lotions), cosmetic hair-treatment preparations (e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, foams, hairsprays, bleaching preparations; e.g. hydrogen peroxide solutions, lightening shampoos, bleaching creams, bleaching powders, bleaching pastes or oils, temporary, semi-permanent or permanent hair colourants, preparations containing self-oxidising dyes, or natural hair colourants, such as henna or camomile).

An antimicrobial soap may have, for example, the following composition:

0.01 to 5% by weight of a polymer, oligomer or molecule of the invention;

0.3 to 1% by weight titanium dioxide;

1 to 10% by weight stearic acid; and

the remainder being a soap base, e.g. the sodium salts of tallow fatty acid and coconut fatty acid or glycerol.

A shampoo may have, for example, the following composition:

0.01 to 5% by weight of a polymer, oligomer or molecule of the invention;

12.0% by weight sodium laureth-2-sulfate;

4.0% by weight cocamidopropyl betaine;

3.0% by weight NaCl; and

water to 100 wt %.

In a sixth aspect of the invention, there is provided an article having a surface, wherein the surface is coated with a polymer or oligomer or pharmaceutically acceptable solvate thereof according to the first aspect of the invention and any technically sensible combination of its embodiments and/or a molecule or pharmaceutically acceptable solvate thereof according to the second aspect of the invention and any technically sensible combination of its embodiments to provide said surface of the article with antimicrobial properties, optionally wherein the article is a urinary catheter.

For example, an article according to the current invention may be a urinary catheter where the surface has been coated by a polymer or oligomer or pharmaceutically acceptable solvate thereof first aspect of the invention and any technically sensible combination of its embodiments and/or a molecule or pharmaceutically acceptable solvate thereof according to the second aspect of the invention and any technically sensible combination of its embodiments. A urinary infection may be caused by a pathogenic bacteria, like E. coli and if this is left untreated, the infection may develop into a systemic infection that may even cause death. By coating the compounds disclosed herein onto a urinary catheter, it can prevent bacterial infection. As will be appreciated, additional components may be added to the coating to provide additional properties (e.g. an anti-inflammatory agent may be coated onto the surface to prevent inflammation etc). As will be appreciated, the antimicrobial compounds disclosed herein can be coated onto other medical devices as well.

Further aspects and embodiments of the invention are described in the following numbered statements.
1. A random copolymer with the following general structure;

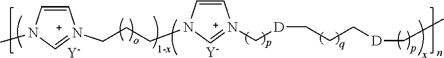

wherein D is a biodegradable fragment which may be amide, ester, carbonate, urethane, disulfide, anhydride, hydrazone;

Y⁻ is a counterion which may be chloride, acetate, phosphate, sulfonate, bis((trifluoromethyl)sulfonyl)imide (N(Tf)₂⁻); and

0≤o≤6;

1≤p≤6;

0≤q≤6.
2. The random copolymer according to Statement 1, wherein xis between 0.10 to 0.50.
3. The random copolymer according to Statement 1 or 2, wherein the random copolymer has a molecular weight distribution of 1 KDa to 5 KDa.
4. A molecule with the following general structure

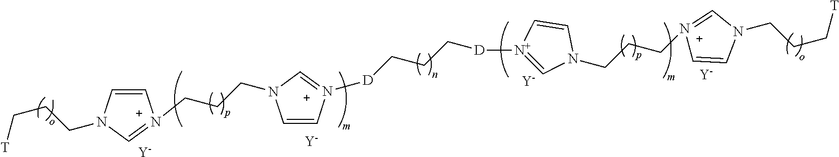

wherein D is a biodegradable fragment which may be amide, ester, carbonate, urethane, disulfide, anhydride, hydrazone;

Y⁻ is a counterion which may be chloride, acetate, phosphate, sulfonate, bis((trifluoromethyl)sulfonyl)imide (N(Tf)₂⁻);

T is a terminal group which may be amine, ammonium, guanidium, bisguanidinium, alkyl, aryl; and

1≤m≤6;

0≤n≤12;

0≤o≤20;

0≤p≤6.
5. Use of a random copolymer as described in any one of Statements 1 to 3, or a molecule as described in Statement 4, in medicine.
6. Use of a random copolymer as described in any one of Statements 1 to 3, or a molecule as described in Statement 4, for use in the treatment of a microbial infection
7. Use of a random copolymer as described in any one of Statements 1 to 3, or a molecule as described in Statement 4, in the manufacture of a medicament to treat a microbial infection in a subject in need thereof.
8. A method of treating a subject suffering from a microbial infection comprising the steps of administering to the subject a therapeutically effective amount of a random copolymer as described in any one of Statements 1 to 3, or a molecule as described in Statement 4, such that the microbial infection is treated.

The antibacterial biodegradable poly- and oligo-imidazoles (plus defined molecules), collectively discussed here as the compounds of the invention, demonstrate good antibacterial activity against both Gram-positive and Gram-negative bacteria in vitro (e.g. the polymer PIM1D and the oligomers OIM1D-mC-6 (m=3, 8)—see experimental section below for further details). Further the compounds of the invention show good in vivo biocompatibility. For example, single intraperitoneal injection of the polymer PIM1D can rescue mice in murine septicaemia model induced by MDR P. aeruginosa and A. baumannii, while accumulative intraperitoneal injection of PIM1D over 7 days caused negligible toxicity. These findings identify the degradable compounds of the invention as promising antibacterial candidates to address the current emerging drug resistance crisis.

Emerging multidrug-resistant bacterial pathogens severely threaten human public health. Antimicrobial polymers were widely explored as alternative therapeutic agents but mostly failed due to their poor biocompatibility and high MIC values. Surprisingly, the compounds of the invention retain high antimicrobial activity while also being biodegradable in vivo, thereby reducing or eliminating problems associated with toxicity of non-degradable compounds in the body, which problems are caused or exacerbated by the prolonged stay of these non-degradable compounds in vivo. For example, the polymer PIM1D showed high antibacterial activity against even multidrug-resistant P. aeruginosa, A. baumannii and K. pneumonia, which are in the WHO's top critical pathogens list. It was also effective against multidrug-resistant Gram-positive bacteria and mycobacterium that's inert to colistin treatment, demonstrating its broad antibacterial efficacy. This, together with its good biocompatibility, make PIM1D a superior antibacterial candidate. Similar properties were found for other compounds of the invention.

Bacterial septicaemia is extremely lethal if not treated. The involvement of multidrug-resistant bacterial pathogens makes treatment even more problematic as they are not treatable by most antibiotics. As noted above and below in the examples, a single injection of PIM1D demonstrated excellent potency in rescuing mice suffering from septicaemia caused by MDR P. aeruginosa PAER and MDR A. baumannii AB-1. PIM1D was also effective in treating murine septicaemia caused by methicillin-resistant S. aureus. Distal lung infection is difficult to treat and usually used to evaluate efficacy of antimicrobial agents before moving to clinical studies. PIM1D showed good efficacy in treating lung infection caused by K. pneumoniae and methicillin-resistant S. aureus. Moreover, only negligible toxicity was observed after 7 consecutive intraperitoneal injections of PIM1D at treatment dose 15 mg/kg, with an accumulative dose of 105 mg/kg. The above highlights the potential of PIM1D (and other compounds of the invention) in antimicrobial applications.

Further aspects and embodiments of the invention will now be described by reference to the following non-limiting examples.

### EXAMPLES

Materials

All chemicals used in the synthesis were purchased from Sigma-Aldrich Co. LLC. (St. Louis, USA) and directly used for reaction, unless otherwise specified. Commercial AR grade solvents were used as received from Merck without further distillation. For column chromatography, technical grade solvents were used as received from SG Labware Pte Ltd (Singapore) without any distillation. 1,4-diaminobutane (Diamine B), mucin, silica gel (35-70 mesh), silica gel 60 (100-200 mesh), Amberlyst® A-26 OH resin, and Cation Adjusted Mueller Hinton Broth (CAMHB) were purchased from Merck & Co., USA. L-lysine and 3,3′-Dipropylthiadicarbocyanine Iodide (DiS-C3-(5)) were purchased from Combi-Blocks, Inc. (San Diego, Calif., USA). N-(3-dimethylaminopropyl)-N′-ethylcarbodiimide hydrochloride (EDC.HCl) and 1-Hydroxybenzotriazole (HOBt) were purchased from GL Biochem Ltd. (Shanghai, China). Cyclophosphamide was purchased from MedChemExpress LLC (Shanghai, China). Propidium iodide (PI) staining kit, Dulbecco's Modified Eagle's Medium (DMEM), fetal bovine serum (FBS), penicillin, streptomycin, N-(2-Hydroxyethyl)piperazine-N′-(2-ethanesulfonic acid) (HEPES) and FM™ 4-64FX were purchased from Thermo Fisher Scientific (MA, USA). 3-(4,5-Dimethylthiazol-2-yl)-2,5-Diphenyltetrazolium Bromide (MTT) was purchased from Alfa Aesar (MA, USA). Fluorescein isothiocyanate (FITC) was purchased from Biotium, Inc. (CA, USA). Pullulan standards were purchased from Polymer Standards Service (PA, USA). 1K Dalton cut-off Spectra/Por®6 dialysis membrane was purchased from Repligen, USA. Muller Hinton Broth (MHB), Trypticase Soy Broth (TSB), Lysogeny Broth (LB) and Agar (LB agar) were purchased from Becton Dickson, USA. Vancomycin and colistin were purchased from Chem-Impex International Inc., USA). Middlebrook 7H9 broth medium was purchased from BD Difco. Bovine serum albumin fraction V was purchased from Roche.

Bacteria and Growth Conditions

Pseudomonas aeruginosa PAO1 was provided by Scott Rice, Nanyang Technology University. Enterococcus faecalis VRE583, and Escherichia coli EC958 were obtained from the Singapore Center for Environmental and Life Sciences (SCELSE). The pan-resistant P. aeruginosa PAER, the pan-sensitive Acinetobacter baumannii ACBAS, the multi-drug resistant A. baumannii AB-1, the pan-sensitive Klebsiella pneumonia KPNS, the carbapenem-resistant K. pneumoniae KPNR, the pan-sensitive E. coli ECOS, the MDR E. coli ECOR, the pan-sensitive Enterobacter cloacae ECLOS, and the carbapenem-resistant E. cloacae CRE were obtained from Tan Tock Seng Hospital (TTSH) Singapore. MRSA USA300 LAC, the LAC derivative S. aureus LAC* and the LAC menD mutant have been described previously (Pader, V. et al., Infect. Immun. 2014, 82, 4337-4347). K. pneumoniae SGH10, K. pneumoniae BAK085, K. pneumoniae M7, K. pneumoniae SGH4, multi-drug resistant A. baumannii X26, extensively drug-resistant A. baumannii X39, A. baumannii X40 were provided by Dr Yunn-Hwen Gan, National University of Singapore. The colistin-resistant P. aeruginosa (PAK pmrB12) and Burkholderia thailandensis 700388 were provided by Samuel I. Miller, University of Washington School of Medicine. Mycobacterium abscessus (rough and smooth) and M. smegmatis mc²155 were cultured and tested in Kevin Pethe's lab in Lee Kong Chian school of medicine. M. bovis bacillus Calmette-Guérin is from our collection. All other bacteria were purchased from the American Type Culture Collection. Unless otherwise specified, bacteria were grown in Mueller Hinton Broth (MHB) (Wiegand, I. et al., Nat. Protoc. 2008, 3, 163-175) at 37° C. with shaking. Staphylococcus aureus was grown on Trypticase Soy Broth (TSB). Mycobacteria were grown in Middlebrook 7H9 broth medium supplemented with 0.2% glycerol, 0.05% Tween 80, and 10% ADS supplement (which was made via dissolving bovine serum albumin fraction V (25 g), D-dextrose (10 g), and sodium chloride (4.05 g) in water (500 mL)). Glycerol was not supplemented for Mycobacterium growth inhibition assay. For plating, we solidified Lysogeny Broth (LB) with 1.5% agar (LB agar) and the plates were incubated at 37° C.

Analytical Techniques

All samples were dissolved in deuterated solvents CDCl₃, D₂O, MeOD or DMSO-d₆ prior to ¹H nuclear magnetic resonance (NMR), ¹H-¹H homonuclear correlation spectroscopy (COSY), heteronuclear multiple quantum coherence (HMQC), ¹³C NMR and distortionless enhancement by polarization transfer (DEPT-135) analysis using a Bruker Avance DPX 300 MHz NMR instrument. In the ¹H NMR spectra, the chemical shift (δ) for solvent residual peaks were set to 4.79 for D₂O, 2.50 for DMSO-d₆, and in proton-decoupled ¹³C NMR spectra, the middle peak for the solvent residual peak of DMSO-d₆ was set to 39.52. Mass analysis was recorded on a MALDI-ToF ABI 4800. Molecular weight and number average molecular weight distribution (M_{w}/Mₙ) were determined by GPC equipped with two ultrahydrogel columns in series and an RI detector using a water/methanol (MeOH)/0.5 M acetic acid (AcOH) mixture (54/23/23 v/v) containing 0.5 M sodium acetate as eluent (pH=4.5, flow rate=0.5 mL/min). All samples were dissolved in acetate buffer at approximately 1 mg/mL and filtered through a 0.22 μm microfilter prior to sample analysis. Merck silica gel 60 (100-200 mesh) was used as the stationary phase for column chromatography separation of crude mixtures. Thin-layer chromatography (TLC) was performed using Merck 60 F254 pre-coated silica gel plates, and the plates were visualized using a UV lamp, chemical staining with ninhydrin, or basic KMnO₄ solution.

Preparation of Acidified Water for Dialysis

Acidified water for dialysis was prepared by adding 1 M hydrochloric acid (HCl, 3 mL) to Millipore water (5 L).

Procedure for Column Loaded with Chloride Anions

A 10% aqueous HCl solution was passed through a glass column packed with Amberlyst® A-26 (OH— form) until the pH of the eluates was the same as the original solution. Then, the resin was washed with water until neutral pH. The process was carried out at room temperature, using gravity as driving force.

Procedure for Gel Filtration Chromatography (GFC)

Sephadex™-G25 powder was dissolved in deionized (DI) water and submerged overnight, where the powder expanded into a slurry. The Sephadex™ slurry was packed into a glass column using deionized (DI) water as the eluent, and gravitational elution.

### Comparative Example 1

### Synthesis of Main-Chain Alkylated Polyimidazolium (PIM) Chloride Salts, PIMO-5

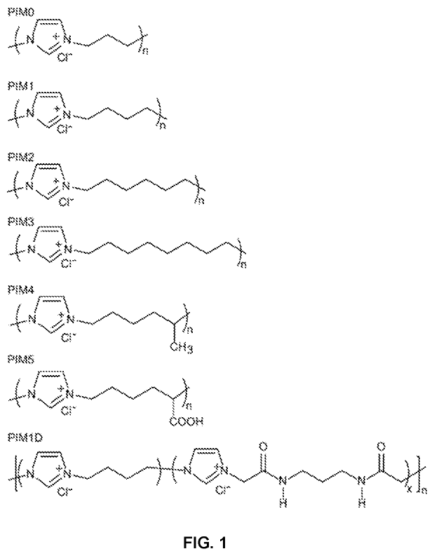

An aqueous acidic solution of a diamine (100 mmol total) selected from the list below was prepared by adding the diamine into water (25 mL) and cooling the reaction mixture in an ice-water bath. After that, 37% HCl solution (200 mmol) was added to the reaction mixture to give the acidic diamine solution. The aqueous acidic solution of the diamine was maintained in an ice water bath for 30 min. After that, a mixture of formaldehyde (8.12 g, 100 mmol) and glyoxal (14.51 g, 100 mmol) was added dropwise to the reaction mixture. The reaction mixture was refluxed for 4.5 h at 80° C. During reflux, the solution changed from colorless to yellowish. The solvent and unreacted monomers were removed by rotary evaporation to give a yellow viscous oil. The oil was diluted with water and dialyzed against acidified water, pH 3-4 (1-KDa-cutoff Spectra/Por®6 dialysis membrane, Repligen, USA) for one day, with the acidified water changed 3 times, to give water-soluble PIMO-5 (FIG. 1).

List of Diamines Used to Synthesize PIMO-5

1,3-diaminopropane—PIMO

1,4-diaminobutane (Diamine B)—PIM1

1,6-diaminohexane—PIM2

1,8-diaminooctane—PIM3

1,5-diamino-2-methylpentane—PIM4

L-lysine—PIM5

PIMO

¹H NMR (300 MHz, D₂O, 25° C. [ppm]): δ 8.98 (s, 1H, imidazole-H), 7.61 (s, 2H, imidazole-H), 4.36 (t, 4H, —CH₂—), 2.54 (m, 2H, —CH₂—).

PIM1

¹H NMR (300 MHz, D₂O, 25° C. [ppm]): δ 8.84 (s, 1H, imidazole-H), 7.51 (s, 2H, imidazole-H), 4.24 (t, 4H, —CH₂—), 1.91 (m, 4H, —CH₂—).

PIM2

¹H NMR (300 MHz, D₂O, 25° C. [ppm]): δ 8.77 (s, 1H, imidazole-H), 7.47 (s, 2H, imidazole-H), 4.16 (t, 4H, —CH₂—), 1.85 (m, 4H, —CH₂—), 1.33 (m, 4H, —CH₂—).

PIM3

¹H NMR (300 MHz, D₂O, 25° C. [ppm]): δ 8.76 (s, 1H, imidazole-H), 7.47 (s, 2H, imidazole-H), 4.16 (t, 4H, —CH₂—), 1.84 (m, 4H, —CH₂—), 1.29 (m, 8H, —CH₂—).

PIM4

¹H NMR (300 MHz, D₂O, 25° C. [ppm]): δ 8.82 (s, 1H, imidazole-H), 7.50 (s, 2H, imidazole-H), 4.19 (t, 2H, —CH₂—), 3.98 (m, 1H, —CH—), 2.15-1.78 (m, 4H, —CH₂—), 1,46-1.15 (m, 2H, —CH₂—), 0.84 (s, 3H, —CH₃).

PIM5

¹H NMR (300 MHz, D₂O, 25° C. [ppm]): δ 9.12-8.78 (m, 1H, imidazole-H), 7.65-7.47 (m, 2H, imidazole-H), 5.13 (m, 1H, —N—CH—), 4.21 (m, 2H, —CH₂—), 2.33-2.18 (m, 2H, —CH₂—), 1.95 (m, 2H, —CH₂—); 1.26 (m, 2H, —CH₂—).

### Comparative Example 2

### Synthesis of PIM1-Fluorescein Isothiocyanate (FITC) Conjugate (FITC-Conjugated PIM1)

PIM1 (1 equiv.) was dissolved in 0.1 M sodium bicarbonate (NaHCO₃) in water (1 mL), and the reaction mixture was stirred for 30 min. After that, FITC (1 equiv.) was added to the reaction mixture and stirred overnight in the dark. The PIM1-FITC conjugate was then dialyzed against acidified water (500-1000 Da cut-off dialysis membrane) for 2 days to remove salts and unreacted dye, with the acidified water changed 3 times per day. The resulting conjugate was lyophilized to obtain the final PIM1-FITC conjugate. The absorbance of PIM1-FITC at 493 nm in PBS was used to establish the calibration curve and from the obtained results, the molar ratio of FITC to PIM1 was estimated to be about 15%.

### Comparative Example 3

### In Vitro Antibacterial and Cytotoxic Effects of PIMO-5

Bacterial Growth-Inhibition and Killing Assays

Minimum inhibitory concentrations (MICs) were determined by a slight modification of the broth microdilution method (Wiegand, I. et al., Nat. Protoc. 2008, 3, 163). Overnight cultures of bacterial strains were subcultured, and grown to mid-Logarithm (Log) phase, followed by optical density (OD) check, then diluted to 1×10⁶ colony-forming unit (CFU) per mL for use as inocula. The test compounds were prepared at a final concentration of 10.24 mg/mL in DI water, and diluted to 1.024 mg/mL in fresh MHB. A two-fold dilution series of the test compound was prepared in MHB media in a 96-well plate (final volume 50 μL per well), achieving a concentration gradient from 512 μg/mL to 1 μg/mL, and incubated at 37° C. with shaking for 10 min (orbital shaker at 225 revolutions per minute (rpm)) prior to inoculation of each well with 50 μL of bacterial suspension, with positive control (MHB media and bacteria suspension without polymer), and sterilized control (only MHB media). The plate was mixed in a shaker incubator for 10 min before incubation at 37° C. for 18 h statically. After which, the OD at 600 nm (OD₆₀₀) was measured. For assays involving Mycobacteria, the compounds were serially diluted in two-fold steps, and 2 μL of this dilution series was spotted in 96-well plates, to which 200 μL of Log-phase bacteria at OD₆₀₀ of 0.005 (about 5×10⁵ CFU/mL) were added. These plates were incubated for 48 h at 37° C. for M. smegmatis, and incubated for 5 days at 37° C. for M. bovis bacillus Calmette-Guérin. The MICs were reported as the concentration of the compound that inhibited bacterial growth by at least 90% (MIC₉₀). Agar plating was done to confirm the seeding bacteria concentration. Three independent experiments were conducted for each compound, and each bacterial strain tested, and the range of MIC values was reported for each compound.

Mammalian Cell Cytotoxicity Assays

Mouse embryonic fibroblast 3T3 cell line was used to test the toxicity of PIMO-5. Cytotoxicity was measured by using a standard method (International Organization for Standardization (2009) ISO 10993-5: Biological evaluation of medical devices-Part 5: Tests for in vitro cytotoxicity. (ISO Geneva), 1-34). 3T3 cells were first cultured in medium containing 89% DMEM, 10% FBS and 1% antibiotics (penicillin/streptomycin). When 80% confluence in the culture flask was observed by microscopy, the cells were treated with trypsin, concentrated and counted using a hemocytometer. 1×10⁴ cells were seeded on each well of a 96-well plate. After incubation of the 96-well plate for 24 h, test compounds with concentration ranging from 128 μg/mL to 4 μg/mL were added to each well of the 96-well plate. After a further 24-h incubation, cell viability was qualitatively evaluated with microscopy and quantified via MTT assay. Cell viability was assessed by comparing the absorbance of formazan in wells with added antimicrobial agents to the absorbance of formazan in wells with untreated cells. ICso values were reported as the test compound level that reduced viable cell number by 50%. The data presented are means of triplicate measurements, and the standard deviations are 10% or less.

LB Agar Plate Counting

The bacterial solution was serial diluted in PBS in a 10-fold manner. The diluted solution was dropped on solidified Agar plate of 5 μL each per drop. After drying in a biosafety hood, the plate was incubated in 37° C. incubator for 18 h, followed by counting of the bacterial colonies and recording of the respective dilution factor. Finally, the bacterial concentration was back-calculated.

Results and Discussion

Table 1 shows the physical and biological properties of varied batches of PIM1 in P. aeruginosa PAER, A. baumannii AB-1 (MDR), and S. aureus USA 300 (MRSA). All of the PIM chloride salts showed significant antimicrobial activity except PIM5 (Table 2). This could be because the carboxylated alkyl chain of PIM5 makes it the least hydrophobic of the series, and that PIM5 is zwitterionic rather than cationic. PIM0 showed reduced activity due to its short alkyl chain being less hydrophobic than PIM1.

TABLE 1
Physical and biological properties of varied batches
of PIM1 in P. aeruginosa PAER, A. baumannii AB-1
(MDR), and S. aureus USA 300 (MRSA).

| | | | MIC₉₀ (μg/ml) | | |
|---|---|---|---|---|---|
| | | | | | S. aureus |
| | Physical property | | P. aeruginosa | A. baumannii | USA300 |
| PIM1 | Mn | PDI | PAER | AB-1 (MDR) | (MRSA) |
| Batch-1 | 1703 | 1.19 | 1 | 2 | 2 |
| Batch-2 | 1592 | 1.16 | 2 | 4 | 2 |
| Batch-3 | 1654 | 1.20 | 1 | 2 | 2 |
| Batch-4 | 1723 | 1.20 | 1 | 2 | 1 |
| Batch-5 | 1510 | 1.15 | 2 | 4 | 2 |

TABLE 2
Antibacterial and cytotoxic effects of PIM0-5.
MIC₉₀ or IC₅₀ (mg per ml) ¹

| | PIM0 | PIM1 | PIM2 | PIM3 | PIM4 | PIM5 |
|---|---|---|---|---|---|---|
| Bacteria² | | | | | | |
| Staphylococcus aureus | 4 | 1-2 | 1-2 | 2 | 4-8 | >256 |
| Enterococcus faecium | 32 | 1-2 | 2-4 | 4 | 8 | >256 |
| Klebsiella pneumoniae | 16 | 2-4 | 1-2 | 2 | 4 | >256 |
| Acinetobacter | 8-16 | 2 | 2 | 4 | 4 | >256 |
| baumannii | | | | | | |
| Pseudomonas | 16 | 2 | 2-8 | 4-8 | 8-16 | >256 |
| aeruginosa | | | | | | |
| Escherichia coli | 32 | 4-8 | 4 | 4 | 8-16 | >256 |
| Enterobacter cloacae | 16 | 2 | 2 | 2-4 | 8 | >256 |
| Mouse cells³ | 155 | >1024 | 206 | 20 | 503 | 525 |

¹The minimum PIM concentration required to inhibit bacterial growth by at least 90% (MIC₉₀) or the half-maximal inhibition (IC₅₀) of 3T3 cell viability. Values are the ranges of three independent experiments.
²The Gram-positive bacterial strains were S. aureus ATCC 29213, E. faecium ATCC 19434, and the Gram-negative bacterial strains were K. pneumoniae ATCC 13883, A. baumannii ATCC 19606, P. aeruginosa PAO1, E. coli ATCC 8739, and E. cloacae ATCC 13047.
³Mouse fibroblast 3T3 cells.

Unlike PIM2 and PIM3, PIM1 did not exhibit toxicity against 3T3 cells (Table 2). This could be due to PIM2 and PIM3 having alkyl chains two or four carbons longer than PIM1, respectively. These results show that small differences in the alkyl chain can affect mammalian cell toxicity dramatically.

Therefore, PIM1 was chosen for further studies due to its potent antibacterial activity across a spectrum of pathogenic bacteria and the fact that it showed no measurable acute mammalian cell toxicity in our screen of the PIMs (Table 2).

### Comparative Example 4

### In Vitro Antibacterial Activity and Cytotoxicity of PIM1

PIM1 was taken to screen for antibacterial activity in a wider variety of bacterial pathogens by following the protocol in Comparative Example 3. The cytotoxicity of PIM1 in HEK293, HepG2 and A549 cells were also determined as described in Comparative Example 3, except that DMEM supplemented with 15% FBS was used for the culturing of HepG2, HEK293 and A549 cells. Additionally, the antibacterial activity of PIM1 was compared to commercial antibiotics, colistin and polymyxin B.

Results and Discussion

We found that PIM1 showed potent antibacterial activity against a variety of pan-antibiotic-resistant Gram-positive and Gram-negative bacteria including colistin-resistant Burkholderia thailandensis and P. aeruginosa mutant. We noted that PIM1 was also a potent anti-Mycobacterium compound. By comparison, PIM1 had a broader activity spectrum than colistin and polymyxin B, which are not particularly effective antibiotics for Gram-positive bacteria (Table 3). These findings suggest that PIM1 has a mode of action distinct from that of colistin. Finally, toxicity was not evident even at the highest PIM1 levels when tested in four different mammalian cell lines (Table 4).

TABLE 3
Antibacterial effect of PIM1 compared to the activity
of colistin on a panel of pan-resistant bacteria
and naturally antibiotic resistant bacteria.

| | MIC₉₀ (mg per mL)¹ | | |
|---|---|---|---|
| Bacteria | PIM1 | Colistin | Polymyxin B |
| S. aureus USA300 (MRSA²) | 2 | >128 | 64-128 |
| S. aureus BAA40 (MRSA) | 2-4 | >128 | 64-128 |
| E. faecalis 583 (VRE²) | 4-8 | >128 | >128 |
| E. coli 958 (MDR) | 4 | 2 | 2 |
| P. aeruginosa PAER | 1 | 1 | 2 |
| P. aeruginosa PAK pmrB12² | 2 | 16 | 32 |
| A. baumanii AB-1 (MDR) | 2-4 | 2 | 2 |
| A. baumanii X26 (MDR) | 2-4 | 2 | 4 |
| A. baumanii X39 (XDR²) | 8 | 4-8 | 8 |
| Burkholderia thailandensis 700388² | 4 | >128 | >128 |
| K. pneumoniae KPNR (MDR) | 2 | 2 | 4 |
| E. cloacae CRE (MDR) | 4 | 2 | 4 |
| Salmonella enterica 13076 | 1 | ND³ | ND |
| Mycobacterium abscessus (rough) | 8-16 | ND | ND |
| Mycobacterium abscessus (smooth) | 8-16 | ND | ND |
| Mycobacterium smegmatis | 2 | ND | ND |
| Mycobacterium bovis bacillus Calmette- | 1 | 128 | 64 |
| Guérin | | | |

¹The concentration of antimicrobial inhibiting bacterial growth by at least 90%. Values are the ranges of three independent experiments.
²MRSA, methicillin-resistant S. aureus; VRE, vancomycin-resistant Enterococcus; MDR, multi-drug resistant; P. aeruginosa PAK pmrB-12 is a colistin-resistant mutant derived from P. aeruginosa PAK (Moskowitz, S. M. et al., J. Bacteriol. 2004, 186, 575-579); XDR, extensive drug resistant (Magiorakos, A. P. et al., Clin. Microbiol. Infect. 2012, 18, 268-281); B. thailandensis 700388 is a naturally colistin-resistant close relative of the emerging pathogen Burkholderia pseudomallei (B. pseudomallei is also colistin resistant) (Olaitan, A. O. et al., Front. Microbiol. 2014, 5, 643).
³ND, not done.

TABLE 4
Comparison of PIM1, colistin and polymyxin B cytotoxicity.

IC₅₀ (μg per mL)¹

| | PIM1 | Colistin | Polymyxin B |
|---|---|---|---|
| Human kidney (HEK293) | >1024 | 64 | 240 |
| Human liver (HepG2) | >1024 | >1024 | 765 |
| Mouse fibroblast (3T3) | >1024 | >1024 | 920 |
| Human epithelial (A549) | 870 | >1024 | 879 |

¹The concentration of antimicrobial that induced the half-maximal inhibition of mammalian cell viability. Values are the averages of triplicates with less than 10% standard deviations.
²ND, not done.

### Comparative Example 5

### Bactericidal Properties of PIM1

To determine whether PIM1 was bactericidal or bacteriostatic, we inoculated MHB with a model Gram-negative pathogen P. aeruginosa PAO1 or TSB with a Gram-positive pathogen methicillin-resistant MRSA S. aureus LAC* from Log-phase cultures, and determined the total CFU in the samples over time in the presence of different concentrations of PIM1 after inoculation was determined by plate counting on LB agar. Two independent experiments were performed, and the results are the means±SD.

Results and Discussion

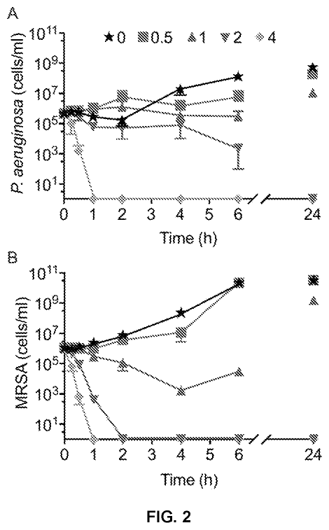

Bacterial growth was evident in the absence of PIM1 or in the presence of PIM1 at a level one-half of the MIC (FIG. 2). At twice the MIC, both P. aeruginosa and S. aureus were killed by PIM1. From these experiments, we concluded that PIM1 is bactericidal.

### Comparative Example 6

### Novel Mode of Action of Antibacterial PIM1

Propidium Iodide (PI) Staining

P. aeruginosa PAO1 was used in the PI experiments. Cells grown in MHB were harvested in mid-Log phase and resuspended in fresh MHB. PIM1 or colistin (positive control) was added at the indicated concentrations. After 1-hour incubation with the antimicrobials, the cell suspensions were sampled to determine cells numbers by plate counting. The remaining cells were washed with PBS and stained with PI (15 μg/mL) according to the manufacturer's protocol. Attune N×T Flow cytometry (Thermo Fisher Scientific, USA) was used to determine the percent of cells that had taken up PI (dead cells). A Zeiss LSM800 confocal microscope was used to image cells on a polylysine-coated petri-dish (MatTek Corporation, USA).

Monitoring Membrane Electric Potential

A membrane potential-sensitive dye, 3,3′-dipropylthiadicarbocyanine iodide (DiS-C3-(5)), was used to monitor membrane electrical potential (Δψ) in P. aeruginosa by using a previously reported procedure (Zhang, L. et al., Antimicrob. Agents Chemother. 2000, 44, 3317-3321). P. aeruginosa PAO1 cells were harvested from mid-Log phase cultures by centrifugation and suspension in 5 mM HEPES buffer containing 100 mM KCl and 0.2 mM EDTA to permeabilize the outer membrane for DiS-C3-(5) entry. The bacterial suspension was then adjusted to an OD₆₀₀ of 0.02 and DiS-C3-(5) was added (final concentration 1 μM). The cell suspension (180 μL) was then added to each well of a 96-well plate, and the test compounds were added to the wells as indicated to bring the final mixture to 200 μL. Fluorescence was measured in each well every 2 min in a Spark 10M microtiter plate reader (Tecan, Switzerland) with excitation at 622 nm and emission 670 nm. Data were collected at 30 min after the addition of the test compound. Two independent experiments were conducted, and the data here are mean values±SD.

Cellular Uptake Protocol

Cellular uptake of PIM1-FITC was monitored as described elsewhere (Radlinski, L. C. et al., Cell Chem. Biol. 2019, 26, 1355-1364) with slight modifications. Briefly, cells grown in MHB were harvested in mid-Log phase, and suspended in fresh MHB containing PIM1-FITC at 1 MIC (the MIC of PIM1-FITC was the same as PIM1) for 30 min. Cells were then harvested by centrifugation, washed once with PBS, and then fixed with 4% paraformaldehyde in PBS for 15 min. Fixed cells were washed twice with PBS, and then incubated with 5 μg/mL FM™ 4-64FX (Invitrogen™, Thermo Fisher Scientific, USA) for 10 min on ice. The cells were washed twice with PBS again, then sealed in slides using Fluoromount™ aqueous mounting medium (Merck & Co., USA), and subsequently imaged using a Zeiss Super Resolution System ELYRA PS.1 with an LSM 800 system.

Results and Discussion

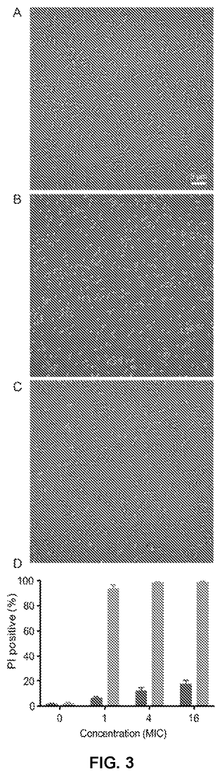

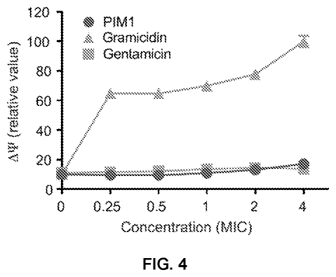

The PIMs were designed to have moderately hydrophobic alkyl chains with cationic imidazolium moieties. Therefore, like antimicrobial peptides (Velkov, T. et al., J. Med. Chem. 2010, 53, 1898-1916), the activity of PIMs could possibly involve permeabilizing cell membranes. Moreover, as seen in Comparative Example 6, PIM1 has a mode of action distinct from that of colistin. To test this hypothesis, the uptake of fluorescent dye PI into PIM1− and colistin-treated P. aeruginosa was compared. Viable cells with intact cell membranes exclude PI. If the membrane is permeabilized, PI can enter cells. As expected, almost all cells treated with colistin were stained but most cells treated even with high concentrations of PIM1 excluded PI (FIG. 3). These results support the view that PIM1 activity does not involve membrane disruption as does colistin. In further support of this view, we used the lipophilic fluorescent dye DiS-C3-(5) to monitor the ALP in P. aeruginosa. While treatment with the proton ionophore gramicidin resulted in a dramatic increase in DiS-C3-(5) fluorescence, indicative of Δψ dissipation, PIM1 did not show such an effect (FIG. 4).

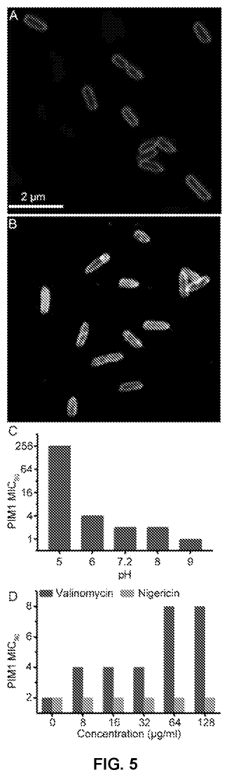

Since PIM1 does not disrupt membranes and does not dissipate Δψ, we speculated that PIM1 might be taken up by cells. Hence, the cellular uptake of a fluorescent derivative of PIM1, PIM1-FITC, was synthesized in Comparative Example 2, and taken to treat P. aeruginosa. As shown in FIG. 5A-B, PIM1-FITC entered cells. We hypothesized that, as is true of cationic antibiotics (for example gentamicin (GEN)), association with cells and antimicrobial activity of PIM1 might depend on Δψ. If so, activity should be high when P. aeruginosa is in alkaline environments, and reduced in acidic environments. In bacteria like P. aeruginosa, the proton motive force (PMF) remains relatively constant over a range of external pH values as does the cytoplasmic pH (mildly basic). The total PMF consists of the Δψ and the pH gradient across the cell membrane (ΔpH). Therefore, in mildly alkaline environments, the cytoplasmic and external pH values are similar, and PMF is primarily in the form of a Δψ. In acidic environments, the outside pH is lower than the cytoplasmic pH, and PMF is primarily in the form of a ΔpH. In fact, PIM1's MIC was dependent on external pH, and PIM1 showed poor antimicrobial activity at pH 5 (FIG. 5C). These findings suggest that PIM 1 uptake is Δψ-dependent.

### Comparative Example 7

### Influence of Valinomycin and Nigericin on MIC of PIM1 Against P. aeruginosa

Valinomycin, nigericin and PIM1 were dissolved in MHB. Stock solutions were added to wells in a microtiter plate to give a volume of 50 μL to which 50 μL of a Log-phase P. aeruginosa culture was added. The MIC₉₀ were determined as described in Comparative Example 3.

Results and Discussion

To gain further insights of the mode of action of PIM1, the influence of potassium ionophore (valinomycin) and sodium-potassium exchanger (nigericin) on PIM1 activity was investigated. At neutral pH, valinomycin reduces Δψ and nigericin collapses ΔpH (Farha, M. A. et al., Chem. Biol. 2013, 20, 1168-1178). The results obtained were consistent with our hypothesis. The MIC of PIM1 for P. aeruginosa was increased by valinomycin treatment but was not affected greatly by nigericin (FIG. 5D). Combining the results obtained here and in Comparative Example 6, we conclude that PIM1 is taken up by cells in a Δψ-dependent manner, but we cannot discern whether it is exerting its antimicrobial effects at the cellular membrane or in the cytoplasm.

### Comparative Example 8

### Influence of Metabolic Status on the Killing of P. aeruginosa PAO1 by PIM1

We used a previously reported method (S. Meylan et al., Cell Chem. Biol. 2017, 24, 195-206) to determine the effect of PIM1 and other antibiotics on the survival of stationary phase P. aeruginosa PAO1, except the stationary-phase cells were obtained by overnight growth in MHB, and we compared PIM1 with GEN. The results were compared to those with P. aeruginosa PAO1 harvested from MHB culture at the mid-Log growth phase. In addition, we tested the ability of an energy source to potentiate PIM1 killing of stationary-phase P. aeruginosa by addition of fumarate (15 mM) to the stationary-phase cells.

Results and Discussion

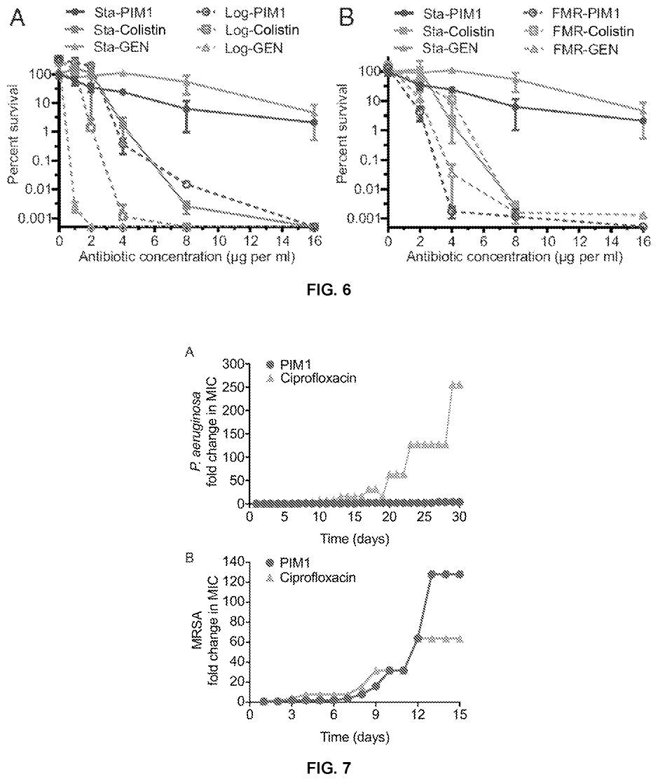

In general, antibiotics have limited activity against nongrowing bacteria. For P. aeruginosa, this is evident when comparing the bactericidal activity of antibiotics, such as GEN on stationary phase cells incubated in the presence and absence of an energy source (S. Meylan et al., Cell Chem. Biol. 2017, 24, 195-206; and K. R. Allison et al., Nature 2011, 473, 216-220). Based on our findings that PIM1 does not appear to disrupt membrane integrity and similar to GEN, it requires Δψ for activity, we hypothesized that its bactericidal activity on nutrient-deprived bacteria might be limited. In fact, stationary phase cells were much less susceptible to PIM1 killing (or GEN killing as a control) than they were to killing by colistin (FIG. 6A). Bactericidal activity of both PIM1 and GEN was restored when fumarate was supplied to the stationary-phase cells as an energy source (FIG. 6B). We conclude that, as is true of GEN and many other antibiotics, PIM1 will have limited utility as a bactericide against nongrowing bacteria. We also note that these experiments are consistent with our conclusion that PIM1 does not act by disrupting cell membranes and that it requires Δψ for activity.

### Comparative Example 9

### Laboratory Evolution of PIM1 Resistance

Laboratory Evolution Mutation Assay

The experiments on the evolution of spontaneous PIM1-resistance and ciprofloxacin-resistance involved sequential passage as described elsewhere (Ling, L. L. et al., Nature 2015, 517, 455-459). We used either P. aeruginosa PAO1 grown in MHB or MRSA LAC* grown in TSB. The inoculum for the initial transfer was 10⁷ cells/mL with varying amounts of antibiotics in 1 mL or 100 μL using 2 mL test tubes and 96-well plates for P. aeruginosa and MRSA, respectively. The larger volumes for experiments with P. aeruginosa were to increase the cell number because resistance did not emerge with smaller culture volumes of this species. The bacteria growth was monitored at 24 h intervals. Transfers were daily, and the inocula for transfers (100-fold dilution) were from the cultures with the highest level of antibiotics that allowed growth to an OD₆₀₀ of at least 0.2. For P. aeruginosa, the experiment was for 30 days. For MRSA LAC*, the experiment was terminated at 15 days. Isolates of MRSA LAC* were obtained from the last transfer, and stored as glycerol stocks at −80° C. for use in further studies.

Whole Genome Sequencing

Standard procedures were used to isolate genomic DNA from PIM1-resistant S. aureus mutants, and the DNA was prepared for sequencing by using an Illumina Nextera DNA Library Preparation Kit. DNA was sequenced on an Illumina MiSeq instrument (paired end sequencing). Sequences were mapped onto the genome of the parent strain MRSA LAC* (Bowman, L. et al., J. Biol. Chem. 2016, 291, 26970-26986), and CLC Genomics Workbench software was used to identify single nucleotide variations, small deletions and insertions. Large deletions were identified by manual sequence comparison. The DNA sequences have been deposited in European Nucleotide Archive (ENA) and the accession number is PRJEB37791.

Results and Eiscussion

To evaluate the potential of designer PIMs as therapeutics and perhaps gain further insights into the PIM mechanism of action, we performed repetitive passaging experiments with escalating concentrations of PIM1 or ciprofloxacin (control) on P. aeruginosa and MRSA. With P. aeruginosa, ciprofloxacin-resistant mutants emerged but PIM1-resistant mutants did not (FIG. 7). PIM1-resistant MRSA emerged at a rate similar to the emergence of ciprofloxacin-resistant mutants.

To gain insights into the nature of the PIM resistance phenotype in our evolved MRSA populations, we isolated the bacteria from the final passage. Out of the 21 isolates characterized, all of them showed a small-colony variant (SCV) phenotype, 15 had PIM1 MICs more than 128 times that of the initial strain, and the other 6 had PIM1 MICs 64-128 times that of the parent strain. We sequenced the genomes of the 15 isolates showing MICs>128 times than the unevolved strain (Shi, Z. et al., Polymer resistant Staphylococcus aureus strains. European Nucleotide Archive. Deposited 14 Apr. 2020.). All but one had a mutation in a gene required for menaquinone biosynthesis (either a gene in the menA-F operon or ispD). Several isolates also had mutations in genes known to confer resistance to cationic peptides, specifically, vraG or vraF, graR or graS, or fmtC (Falord, M. et al., PloS One 2011, 6, e21323; Joo, H.-S. et al., Biochim. Biophys. Acta 2015, 1848, 3055-3061; and Yang, S.-J. et al., Infect. Immun. 2012, 80, 74-81) (Table 5). The genes coding for menaquinone synthesis were of particular interest because a relationship between menaquinones and PIM1 activity might provide some clue about the mode of PIM1 action. Therefore, we compared PIM1 susceptibility of a menD deletion mutant to its parent.

This menD mutant cannot make menaquinone (Lannergård, J. et al., Antimicrob. Agents Chemother. 2008, 52, 4017) and is growth-restricted to fermentation. Like our evolved PIM1-resistant isolates, this mutant has a SCV phenotype. This is a characteristic phenotype of menaquinone synthesis mutants (Von Eiff, C. et al., J. Bacteriol. 2006, 188, 687). The menD mutant showed an 8-fold increase in PIM1 resistance over its parent (MIC of 16 μg/mL vs 2 μg/mL for the parent). Thus, we believe that menaquinones or a functional electron transport system are involved in the susceptibility of MRSA to PIM1, but other factors must also be involved in the very high PIM1 resistance of our evolved isolates. We reasoned that either PIM1 directly interferes with the electron transport chain that leads to generation of toxic reactive oxygen species, or that during fermentative growth, PIM1 uptake is hindered and thus its antimicrobial activity is diminished.

TABLE 5
List of common relevant mutations in laboratory-
evolved PIM1-resistant S. aureus LAC* mutants.

| Mutant | Genes and mutations¹ |
|---|---|
| 4945 | ispD +1 A at A145 |
| 4946 | ispD +1 A at A145, graR T37G |
| 4947 | menE ΔC101, graR T37G |
| 4948 | menE ΔC101, ispD G47A |
| 4949 | ispD G47A and +1 A at A145, menB G365A |
| 4950 | ispD G47A and +1 A at A145, menB G365A |
| 4951 | menE C941T, graR A31G |
| 5103 | menF G665T |
| 5104 | menD C860A, fmtC C287T |
| 5105 | menD ΔA1573, fmtC C148T |
| 5106 | menE C176T, fmtC G182A |
| 5111 | menE G615A, graR A551T, fmtC C148T |
| 5112 | ispD C619T, fmtC C884T, vraF T583C |
| 5113 | menA C55A |
| 5114² | rpoF G172T, cobI T356C, tarL G935A |

¹All single base substitutions were non-synonymous mutations coding for either an amino acid substitution or a stop codon.
²Mutant 5114 is the only PIM1-resistant mutant for which we did not identify a mutation in a menaquinone biosynthesis gene.

### Comparative Example 10

### Efficacy of PIM1 Treatment in an Animal Infection

Mice were housed for one week in a 12-h light-dark cycle at room temperature prior to infection. Our skin infection model was as follows: wounds (diameter about 5 mm) on the shaved dorsal skin of female C57B/6 mice (8-9 weeks of age) were created by punch biopsy, and Log-phase cells of P. aeruginosa PAER were introduced into the wound (about 10⁶ CFU in 10 μL PBS) by pipetting. The infected wounds were immediately covered with Tegaderm (3M, USA). At 4 h post-infection, antimicrobial (PIM1 and Imipenem (Imp)) treatment was initiated by injection through the Tegaderm. After that, another layer of Tegaderm was applied. After a further 24 h, we removed a 1-cm² square tissue sample from the center of a wound, homogenized the sample and determined cell numbers by plate counting. Our protocol was approved by the Institutional Care and Use Committee of Nanyang Technological University (NTU IACUC, protocol A0362).

Results and Discussion

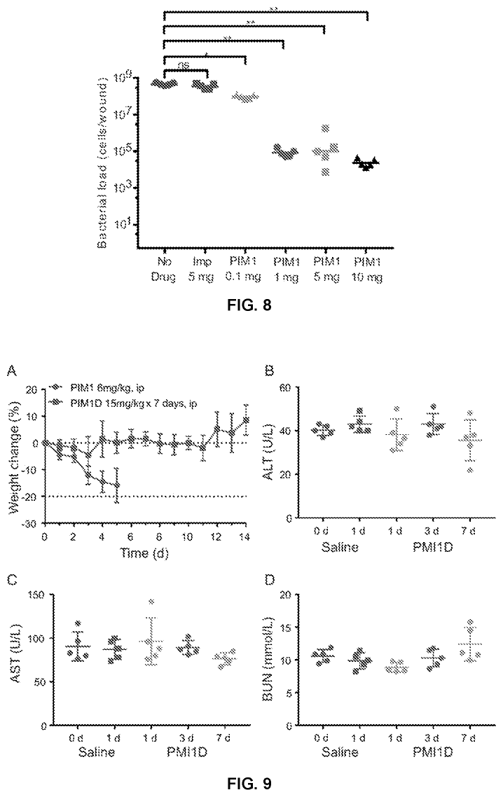

The ability of PIM1 to control a carbapenem-resistant P. aeruginosa murine wound infection was evaluated. As expected, the Imp-resistant strain of P. aeruginosa increased in numbers over the next 24 h in untreated or Imp-treated wounds. In comparison to untreated or Imp-treated wounds, P. aeruginosa numbers were slightly reduced when treated once with PIM1 at 0.1 mg/kg, and were substantially reduced by about four logarithms when treated once with PIM1 at a dose of 1 mg/kg or above (FIG. 8).

### Comparative Example 11

### Toxicity of PIM1 Treatment in an Animal Infection

For the systemic infection model, we first assessed toxicity of PIM1 (IP injection, 6 mg/kg) in female BALB/c mice (8-9 weeks of age) by following weight over a period of 14 days. Weights were recorded daily for 5 days.

Results and Discussion

The safety of PIM1 when delivered to mice by IP injection was tested, and evidence of acute toxicity was found. We observed a decrease in body weight over a period of 5 days after administration of a single dose (FIG. 9A).

### Example 1

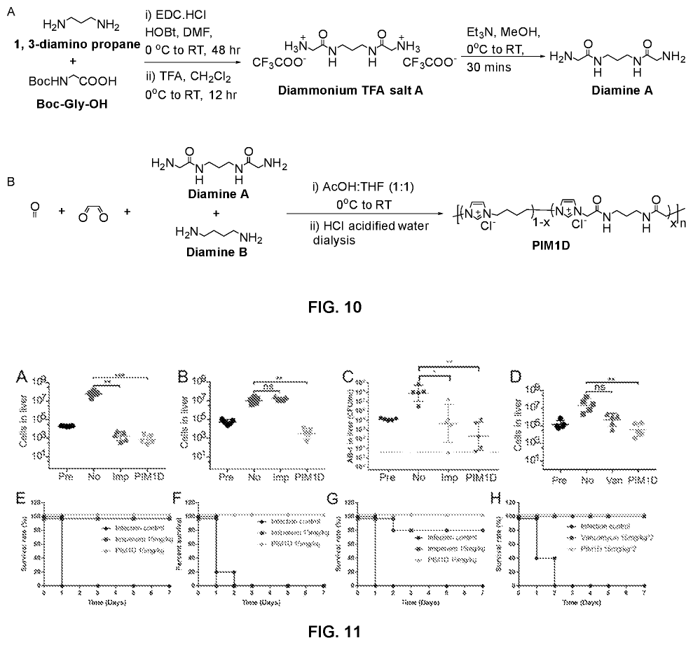

### Synthesis of Precursor of Degradable PIM1D, (N, N′-(propane-1,3-diyl)bis(2-aminoacetamide)) (Diamine A) (FIG. 10A)

EDC.HCl (14.58 g, 76.1 mmol) and HOBt (10.70 g, 79.14 mmol) were added to a solution of Boc-Gly-OH (8.0 g, 45.66 mmol) in anhydrous DMF (25 mL), at 0° C. (ice water) with stirring over 30 min. 1,3-diaminopropane (1.28 mL, 15.22 mmol) kept at room temperature was added dropwise to the reaction mixture kept at 0° C. (ice water) over 10 min. Then, the reaction mixture was brought to room temperature and continuously stirred for 48 h. Water (50 mL) was then added and the product was extracted thrice with ethyl acetate (EtOAc) or DCM (150 mL) The extracts were washed with water (50 mL) thrice, and then washed once with brine (50 mL). The EtOAc or DCM layer was dried with anhydrous Na₂SO₄ (approx. 50 g). Na₂SO₄ was then filtered away and the filtrate was concentrated by rotary evaporation (20 min at 50° C., 120 rpm). The residue was dried under vacuum overnight at room temperature. The dried residue was dissolved in anhydrous DCM (30 mL) then kept at 0° C. (ice water) and trifluoroacetic acid (TFA, 8 mL) was added dropwise over 10 min. After that, the reaction mixture was stirred at room temperature for 12 h. The crude product was concentrated by rotary evaporation at 50° C. for 10 min at 120 rpm. Then, toluene (50 mL) was added and the solution was subjected to further rotary evaporation at 50° C. for 30 min at 120 rpm. The residue was purified by silica gel 60 column chromatography with successive eluents of (i) 30% methanol (MeOH) in dichloromethane (DCM, 500 mL) to remove impurities, followed by (ii) 2% TFA in MeOH (1000 mL), to yield the degradable diammonium TFA salt A (3.0 g, 7.20 mmol).

¹H NMR (300 MHz, DMSO-d₆, 25° C. [ppm]): δ 8.55 (t, J=5.4 Hz, 2H), 8.18 (brs, 6H), 3.53 (s, 4H), 3.14 (q, J=6.3 Hz, 4H), 1.54-1.63 (m, 2H). ¹³C NMR (75 MHz, DMSO-d₆, 25° C. [ppm]): δ 166.14, 159.58 (—CO—CF₃), 159.16 (—CO—CF₃), 158.74 (—CO—CF₃), 158.32 (—CO—CF₃), 123.24 (—CF₃), 119.29 (—CF₃), 115.33 (—CF3), 111.38 (—CF₃), 40.26, 36.73, 28.86.

### Example 2

### Synthesis of Degradable PIM1D

To obtain diamine A, Et₃N (1 mL) was added to a stirred solution of diammonium TFA salt A (400 mg, 0.96 mmol) in MeOH (4 ml) maintained at 0° C. (ice water). After stirring the reaction mixture for 30 min at room temperature, the volatiles were evaporated under rotary evaporator, and dried under vacuum at room temperature for 20 min to afford the degradable diamine, A. The obtained diamine A was immediately used for poly-Radziszewski reaction with diamine B to form biodegradable PIM 1D.

The synthesis of PIM1D was carried out as depicted in FIG. 10B. A first mixture of glyoxal (40 wt %, 349 mg, 2.4 mmol) and formaldehyde (37 wt %, 195 mg, 2.4 mmol) in glacial AcOH and tetrahydrofuran (THF) (3:1.25 mL) at 0° C. (ice water) was prepared. A second solution comprising of degradable Diamine A (181 mg, 0.96 mmol) and nondegradable Diamine B (127 mg, 1.44 mmol) in AcOH and THF (3:1.25 mL) at 0° C. (ice water) was also prepared. The first mixture was added dropwise to the second mixture over 10 min at 0° C. (ice water). Then, the reaction mixture (which was yellowish in color) was allowed to warm to room temperature, and the reaction mixture turned to a brown colour. After allowing the reaction mixture to sit for 24 h at room temperature, the final reaction mixture (around 10 mL) was directly transferred into a 1K Dalton cut-off Spectra/Por®6 dialysis membrane (Repligen, USA), and dialyzed against 5 L of acidified water (pH=3-4), and the acidified water was replaced 3 times over a 24 h duration. The polymer solution in the dialysis bag was transferred to a round bottomed flask, and water was evaporated with a rotary evaporator (70° C., 1 h, 120 rpm) to give a solid PIM1D in the round bottomed flask. To transfer the PIM1D for freeze-drying, water (5 mL) was added to polymer solution and the concentrated PIM1D solution was decanted into a small falcon tube (15 mL), then freeze-dried at −80° C. to afford pure PIM1D. To confirm the molecular weight and chemical structure of PIM1D, GPC and NMR characterization were performed.

Characterization

GPC showed narrow distribution of the final PIM1D compound. Chemical shifts at 9.62 ppm and 7.81 ppm in ¹H NMR spectrum in DMSO-d₆ confirmed the formation of imidazolium ring, while the signals at 1.59 to 5.06 ppm correspond to the alkyl chain in PIM1D. ¹³C NMR spectrum in DMSO-d₆ further confirmed the peak assignments: signals from 121.06 to 136.53 ppm indicate the imidazolium ring formation, signals from 25.78 to 52.77 ppm indicate the presence of alkyl chain, and signals at 164.99 and 167.05 ppm indicate the presence of amide carbonyls. These assignments were further confirmed via DEPT-135, COSY and HMQC analyses. In DEPT spectra, the amides' carbonyl group signals, which appeared at 167.05 and 164.99 ppm in ¹³C NMR, disappeared, and the signals corresponding to the C2-H, C4-H and C5-H protons of the imidazolium ring showed positive phase while the other signals for the CH₂ groups of the polymer chain showed negative phase. In COSY spectra, correlation of alkyl chain in polymer chain was observed, indicating their adjacent positions. However, correlations did not appear for the signals at 5.07 ppm and 4.57 ppm, confirming that the two nonequivalent —CH₂—CO— groups have no adjacent protons, indicating —CH₂— carbon of these groups is attached to N-atom of imidazolium ring. The HMQC spectrum further corroborates the assignments by showing correlation of protons and carbons in both imidazolium rings and alkyl chains of PIM1D.

### Example 3

### Optimization of the Reaction Conditions for PIM1D Synthesis

To optimize the effect of reaction conditions on the biological profile of PIM1D, specific reaction parameters in Example 2 were changed, including feeding ratio of diamine A to diamine B, temperature of reaction, reaction time, and etc. The biological profile of PIM1D was determined by its antibacterial activity and cell viability as described in Comparative Example 3.

Results and Discussion

The molar ratios of diamine A to diamine B (Table 6, entries 1-3) was varied to optimize the percentage of the degradable portion (diamine A). The results show that entry 3 (with 2:3 molar ratio of diamine A to diamine B in the feed) produced an optimized PIM1D with good antibacterial efficacy and lowest mammalian cell toxicity (Tables 7-8, entry 3). Table 6, Entries 1-2 (and corresponding entries 1-2, Tables 7-8), with lower degradable diamine feed ratio, resulted in PIM1Ds that were much more toxic but showed good antibacterial efficacy.

TABLE 6
Reaction conditions optimization for PIM1D synthesis from diamine A and diamine B.

| | Molar | | | | | | | |
|---|---|---|---|---|---|---|---|---|
| | ratios of | Reaction and dialysis condition | | | | | | |
| | diamine | | | | Mol. Wt | | | ¹H NMR |
| | A and | Solvent | | | Cut-off | | | Data |
| | diamine | ratio | Temperature | Reaction | Dialysis | Dialysis | Counter | “a” and |
| Entry | B | AcOH:THF | of reaction | time | membrane | time | ion | “b” ratios |
| 1 | 1:4 | 3:1.25 | RT | 24 hr | 1K | 24 hr | Cl⁻ | 10.1:89.9 |
| 2 | 1.5:3.5 | 3:1.25 | RT | 24 hr | 1K | 24 hr | Cl⁻ | 14.7:85.3 |
| 3 | 2:3 | 3:1.25 | RT | 24 hr | 1K | 24 hr | Cl⁻ | 21.5:78.5 |
| 4 | 2:3 | 3:1.25 | RT | 28 hr | 1K | 24 hr^{a} | Cl⁻ | 22.8:77.2 |
| 5 | 2:3 | 1:1 | RT | 1 hr | 0.5-1K | 44 hr | Cl⁻ | 20.6:79.4 |
| 6 | 2:3 | 1:1 | RT | 2 hr | 0.5-1K | 40 hr | Cl⁻ | 18.7:81.3 |
| 7 | 2:3 | 1:1 | RT | 18 hr | 0.5-1K | 48 hr | Cl⁻ | 18.0:82.0 |
| 8 | 2:3 | 1:1 | RT | 24 hr | 0.5-1K | 48 hr | Cl⁻ | 17.3:82.7 |
| 9 | 2:3 | 3:1.25 | 35° C. | 24 hr | 1K | 24 hr | Cl⁻ | 20:80 |
| 10 | 2:3 | 1:0 | RT | 24 hr | 0.5-1K | 48 hr | AcO⁻ ^{b,c} | 22.3:77.7 |
| 11 | 2:3 | 3:1.33 | RT | 24 hr | 0.5-1K | 48 hr | AcO⁻ ^{c} | 25.4:74.6 |
| 12 | 2:3 | 3:1.33 | RT | 24 hr | 0.5-1K | 48 hr | Cl⁻ | 23.0:77.0 |
| 13 | 2:3 | 3:1 | 80° C. | 16 hr | 1K | 24 hr | Cl⁻ | 23.6:76.4 |
| 14 | 2:3 | 3:1 | RT | 24 hr | 1K | 20 hr | Cl⁻ | 25.3:74.7 |
| 15 | 2:3 | 3:1 | RT | 48 hr | 1K | 20 hr | Cl⁻ | 27.0:73.0 |
| 16 | 2:3 | 3:1 | 60° C. | 24 hr | 1K | 24 hr | Cl⁻ | 25.3:74.7 |
| 17 | 2:3 | 3:1 | RT | 24 hr | 1K | 22 hr | Cl⁻ | 24.2:75.8 |

*The reactions and purification under different conditions (as detailed in Table 1) were performed according to the typical experimental procedure given for PIM1D synthesis.
^{a}Reaction performed at bigger scale (4.8 mmol aldehydes scale).
^{b}Reaction was performed with high dilution (AcOH (10 mL) for 2.4 mmol of aldehyde).
^{c}Polymers containing acetate counter ions were obtained without using HCl during dialysis.

TABLE 7
Antibacterial activity of PIM1D synthesized under varying reaction conditions.

| | GPC | | | | MIC₉₀ (μg/ml) | | | |
|---|---|---|---|---|---|---|---|---|
| Entry | Mn | Mw | PDI | Counter ion | PAO1 | MDR PAER | MDR AB-1 | MRSA USA300 |
| 1 | 3777 | 5360 | 1.41 | Cl⁻ | 2 | 4 | 8 | 4 |
| 2 | 2278 | 3235 | 1.42 | Cl⁻ | 2 | 4 | 8 | 4 |
| 3 | 1918 | 2620 | 1.36 | Cl⁻ | 2 | 4 | 16 | 8 |
| 4 | 2706 | 3330 | 1.23 | Cl⁻ | 8 | 8 | 16 | ND |
| 5 | 1010 | 1399 | 1.38 | Cl⁻ | 8 | 8 | 4 | 16 |
| 6 | 1061 | 1482 | 1.39 | Cl⁻ | 8 | 8 | 8 | 8 |
| 7 | 1077 | 1544 | 1.43 | Cl⁻ | 8 | 8 | 8 | 16 |
| 8 | 1133 | 1622 | 1.43 | Cl⁻ | 8 | 8 | 8 | 16 |
| 9 | 1689 | 2273 | 1.34 | Cl⁻ | 8 | 8 | 8 | 8 |
| 10 | 1287 | 1969 | 1.52 | AcO⁻ | 8 | 8 | 8 | 8 |
| 11 | 1365 | 2177 | 1.59 | AcO⁻ | 8 | 16 | 16 | 8 |
| 12 | 1468 | 2340 | 1.59 | Cl⁻ | 4 | 8 | 8 | 8 |
| 13 | 1919 | 2730 | 1.42 | Cl⁻ | 8 | 8 | 32 | 8 |
| 14 | 1673 | 2210 | 1.32 | Cl⁻ | 8 | 8 | 16 | 8 |
| 15 | 1643 | 2155 | 1.31 | Cl⁻ | 8 | 8 | 16 | 8 |
| 16 | 1767 | 2291 | 1.59 | Cl⁻ | 8 | 8 | 8 | 8 |
| 17 | 1856 | 2411 | 1.29 | Cl⁻ | 8 | 8 | 8 | 8 |

ND, not determined.

TABLE 8
Cell viability of PIM1D synthesized under varying reaction conditions.

| | | | | | Cell viability (%) | | | | | | | |
|---|---|---|---|---|---|---|---|---|---|---|---|---|
| | | | | | 3T3 cells | | | | HepG2 cells | | | |
| | GPC | | | Counter | 1024 | 512 | 256 | 128 | 1024 | 512 | 256 | 128 |
| Entry | Mn | Mw | PDI | ion | μg/ml | μg/ml | μg/ml | μg/ml | μg/ml | μg/ml | μg/ml | μg/ml |
| 1 | 3777 | 5360 | 1.41 | Cl⁻ | 3.90 | 4.17 | 5.16 | 9.96 | ND | ND | ND | ND |
| 2 | 2278 | 3235 | 1.42 | Cl⁻ | 13.69 | 29.73 | 48.78 | 86.73 | ND | ND | ND | ND |
| 3 | 1918 | 2620 | 1.36 | Cl⁻ | 70.81 | 90.30 | 91.84 | 98.96 | ND | ND | ND | ND |
| 4 | 2706 | 3330 | 1.23 | Cl⁻ | ND | 68.26 | 78.64 | 92.76 | ND | ND | ND | ND |
| 5 | 1010 | 1399 | 1.38 | Cl⁻ | 60.17 | 67.09 | 73.33 | 81.90 | 51.97 | 62.81 | 70.54 | 87.35 |
| 6 | 1061 | 1482 | 1.39 | Cl⁻ | 68.51 | 81.77 | 90.75 | 94.16 | 75.65 | 88.61 | 94.59 | 98.49 |
| 7 | 1077 | 1544 | 1.43 | Cl⁻ | 94.95 | 95.23 | 97.77 | 99.60 | 88.75 | 98.43 | 99.45 | 101.60 |
| 8 | 1133 | 1622 | 1.43 | Cl⁻ | 86.96 | 92.87 | 98.25 | 98.30 | 97.58 | 99.42 | 99.69 | 101.66 |
| 9 | 1689 | 2273 | 1.34 | Cl⁻ | 65.55 | 80.57 | 92.69 | 100.69 | 74.62 | 81.98 | 87.69 | 94.31 |
| 10 | 1287 | 1969 | 1.52 | AcO⁻ | 66.94 | 90.62 | 98.27 | 101.53 | 20.73 | 83.74 | 98.18 | 98.61 |
| 11 | 1365 | 2177 | 1.59 | AcO⁻ | 89.70 | 97.10 | 99.11 | 100.40 | 96.46 | 99.20 | 99.12 | 99.55 |
| 12 | 1468 | 2340 | 1.59 | Cl⁻ | 65.05 | 93.99 | 100.96 | 101.04 | 21.91 | 91.46 | 97.77 | 98.53 |
| 13 | 1919 | 2730 | 1.42 | Cl⁻ | 96.71 | 97.10 | 98.02 | 98.99 | 19.65 | 89.42 | 97.93 | 99.19 |
| 14 | 1673 | 2210 | 1.32 | Cl⁻ | 90.61 | 94.48 | 98.83 | 100.21 | 93.96 | 98.62 | 98.81 | 99.70 |
| 15 | 1643 | 2155 | 1.31 | Cl⁻ | 53.01 | 66.93 | 85.93 | 99.20 | 74.51 | 84.57 | 92.48 | 99.21 |
| 16 | 1767 | 2291 | 1.59 | Cl⁻ | 94.37 | 94.49 | 94.73 | 96.55 | 65.67 | 89.81 | 93.27 | 94.65 |
| 17 | 1856 | 2411 | 1.29 | Cl⁻ | 82.08 | 94.35 | 94.25 | 97.32 | 92.81 | 95.54 | 96.47 | 97.93 |

ND, not determined.

Further reaction conditions optimization was conducted by varying solvent ratios, temperature, time of the polymerization reaction, dialysis membrane and dialysis time (Table 6, entries 4-17). The obtained compounds showed Mₙ in the range of 1 KDa to 2 KDa with narrow molecular weight distribution, and the final percentage of degradable diamine A (in the products) was in the range of 17% to 30% (Table 6, entries 4-17). All these compounds showed good antibacterial activity, with MIC₉₀ mostly in the range of 4-16 μg/mL against both MDR P. aeruginosa and methicillin-resistant S. aureus (Table 7, entries 4-17). The biocompatibility was tested using 3T3 fibroblast cells and liver HepG2 cells, and the tested compounds (Table 8, entry 4-17) showed cell viability above 50% at all four concentrations (128 μg/mL to 1024 μg/mL). These results indicate that slight modifications of the reaction conditions in PIM1D synthesis do not greatly affect its biological properties (Tables 6-8, entries 4-17), and the biological profile of PIM 1D is not sensitive to molecular weight in the range of 1 KDa to 2 KDa. This tolerance to variations in reaction conditions would make the development of the compound into commercial products easier, thus it has great potential in varied antimicrobial agent applications.

### Example 4

### In Vitro Antibacterial Activity and Biocompatibility of PIM1D

PIM1D and colistin were tested against a larger panel of MDR Gram-positive and Gram-negative bacteria by following the protocol in Comparative Example 3. In vitro biocompatibility of PIM1D and colistin were evaluated via MTT tests using 3T3, HEK293, HepG2 and A549 cells by following the protocol in Comparative Example 3.

Results and Discussion

Table 9 shows the physical and biological properties of varied batches of PIM1D in P. aeruginosa PAER, A. baumannii AB-1 (MDR), and S. aureus USA 300 (MRSA). Surprisingly, PIM1D showed potent antibacterial activity towards a larger panel of MDR Gram-positive and Gram-negative bacteria including intrinsically colistin-resistant B. Thailandensis 700388 (Table 10), MDR A. baummannii, P. aeruginosa and K. pneumoniae that are top critical pathogens for which WHO demands new antibiotics (World Health Organization (WHO). Global priority list of antibiotic-resistant bacteria to guide research, discovery, and development of new antibiotics. 2017). We note that PIM1D is a potent anti-Mycobacterium compound as well. Overall, we demonstrated that PIM1D is an effective antibacterial agent and has broader spectrum activity than colistin.

TABLE 9
Physical and biological properties of varied batches
of PIM1D in P. aeruginosa PAER, A. baumannii AB-1
(MDR), and S. aureus USA 300 (MRSA).

| | | | MIC₉₀ (μg/ml) | | |
|---|---|---|---|---|---|
| | | | | | S. aureus |
| | Physical property | | P. aeruginosa | A. baumannii | USA300 |
| PIM1D | Mn | PDI | PAER | AB-1 (MDR) | (MRSA) |
| Batch-1 | 1689 | 1.34 | 8 | 8 | 8 |
| Batch-2 | 1673 | 1.32 | 8 | 16 | 8 |
| Batch-3 | 1643 | 1.31 | 8 | 16 | 8 |
| Batch-4 | 1767 | 1.59 | 8 | 8 | 8 |
| Batch-5 | 1856 | 1.29 | 8 | 8 | 8 |

TABLE 10
MIC₉₀ and cytotoxicity of PIM1D against pathogens,
mycobacteria and human cell lines.

| | Concentration (μg/ml) | |
|---|---|---|
| Organism and genotype | PIM1D | Colistin |

Pathogenic bacteria (MIC₉₀)

| | | |
|---|---|---|
| S. aureus USA300 (MRSA) | 4 | >128 |
| S. aureus BAA40 (MRSA) | 2 | >128 |
| E. faecalis 583 (VRE) | 8 | >128 |
| E. coli 958 (MDR) | 16 | 2 |
| P. aeruginosa PAO1 | 8 | 2 |
| P. aeruginosa PAER (MDR) | 4 | 1 |
| P. aeruginosa PAES (clinical) | 4 | 2 |
| P. aeruginosa PAK pmrB12 | 4 | 16 |
| A. baumannii AB-1 (MDR) | 4 | 2 |
| A. baumannii X26 (EDR) | 8-16 | 2 |
| A. baumannii X39 (EDR) | 8 | 4-8 |
| A. baumannii X40 (EDR) | 8 | 8 |
| B. Thailandensis 700388 | 16 | >128 |
| K. pneumoniae SGH10 (clinical strain) | 8 | 2 |
| K. pneumoniae KPNR (MDR) | 8 | 2 |
| K. pneumoniae BAK085 (MDR) | 8 | 2 |
| K. pneumoniae M7 (MDR) | 8 | 16-32 |
| E. cloacae ECLOS (MDR) | 4 | 16 |
| E. cloacae CRE (MDR) | 8 | 2 |
| Salmonella enterica 13076 | 1 | ND |

Mycobacteria (MIC₉₀)

| | | |
|---|---|---|
| Mycobacterium abscessus (rough) | 32-64 | ND |
| Mycobacterium abscessus (smooth) | 32-64 | ND |
| Mycobacterium smegmatis | 2 | ND |
| Mycobacterium bovis bacillus Calmette-Guérin | 0.5 | 128 |

Human cell line (IC₅₀)

| | | |
|---|---|---|
| Human fibroblast (3T3) | >1024 | >1024 |
| Human liver (HepG2) | >1024 | >1024 |
| Human kidney (HEK293) | 716 | 64 |
| Human epithelial (A549) | 870 | >1,024 |

ND, not determined, no data.
MRSA, Methicillin-resistant staphylococcus aureus.
MDR, multidrug-resistant.
EDR, extensive drug-resistant.

PIM1D showed IC₅₀ values larger than 1024 μg/mL which are in a similar range as the antibiotics control, colistin (Table 10). Considering that the MIC₉₀ value of PIM1D against most bacterial strains was in the range of 8-16 μg/mL, it would have a large therapeutic window of more than 50. Therefore, PIM1D has potential to be developed into an antimicrobial agent.

### Example 5

### In Vivo Toxicity and Antimicrobial Efficacy of Degradable PIM1D

In Vivo Toxicity Study

The in vivo toxicity of PIM1D was assessed by monitoring mice weight and blood biomarkers over a period of 14 days. The BALB/c female mice (8-9 weeks of age) were randomly grouped into two groups: saline control and PIM1D-treated group. Each mouse in the PIM1D-treated group received PIM1D (15 mg/kg) daily for seven consecutive days via IP injection (accumulative dose of 105 mg/kg). The same volume of saline was intraperitoneally injected in saline control group. At day 1, day 3 and day 7, mice blood was withdrawn from submandibular vein to perform blood biochemistry assay using a Pointcare V3 Blood Chemistry Analyzer (MNCHIP, Tianjin, China) according to manufacturer's protocol (Zhang, K. et al., Nat. Commun. 2019, 10, 4792). Similarly, mice blood in saline control group was collected and quantified for comparison. The mice condition were monitored closely till 14 days after first injection. The protocol was approved by Animal Ethics and Welfare Committee (AEWC, protocol AEWC-2018-07) of Ningbo University.

In Vivo Efficacy Study

Murine septicemia model was used to evaluate the in vivo efficacy of PIM1D. The experiment for mice septicemia infection model of MDR P. aeruginosa PAER and MDR A. baumannfi AB-1 was conducted within guidance of approved protocol by the Institutional Care and Use Committee of Nanyang Technological University (NTU IACUC). The experiments for murine septicemia infection model of wild type P. aeruginosa PAO1 and methicillin-resistant S. aureus MRSA USA300 were conducted following protocol reviewed and approved by Animal Ethics and Welfare Committee (AEWC) of Ningbo University. BALB/c female mice (8-9 weeks of age) were used to test the septic shock protection efficacy for all murine infection models. Exponential phase bacteria were washed twice with saline, and re-suspended into the same volume of saline. 300 μL bacterial suspension with varied concentration in 5% mucin was introduced into each mouse via IP injection to first determine the lethal bacterial dosage, and the determined concentration was then used in the following study. The use of mucin is to endow mice immunocompromised, as similar to hospitalized patients. At 2 h post-infection, mice (5 per group) were treated with a single dose of the test compound. Positive and negative control groups of mice were injected with, respectively, the same dose of antibiotics and the same amount of saline, at the same time point. Mouse survival was monitored over 7 days. In a separate set of mice, all mice were euthanized at 26 h post-infection. Peritoneal washes were then performed by injecting PBS (2.0 mL) into the IP cavity, followed by 1 min of abdomen massage. Then, around 0.5 mL of peritoneal fluid was recovered for CFU analysis. Bacterial loads were also evaluated in the spleen, liver and kidney of the animal. To check whether bacterial infection is established 2 h post-infection, mice which received the same bacterial inoculum were sacrificed, and the IP fluid as well as all organs (including kidney, liver and spleen) were harvested to determine CFU. Experiments on septicemia caused by MRSA were similar to those of P. aeruginosa except the mice were immunosuppressed by intraperitoneally injecting 150 mg/kg and 100 mg/kg cyclophosphamide at day 4 and day 1 (Chin, W. et al., Nat. Commun. 2018, 9, 917). Treatments were given twice, at 2 h and 26 h after infection. Bacteria loads were from organs in mice that were sacrificed 50 h post-infection. For the non-treatment group, the mice were sacrificed 26 h or 50 h post-infection, whichever is closer to their death time. For the pre-treatment group, mice were sacrificed 2 h after infection. The bacterial levels were analyzed with one-way classification analysis of variance (ANOVA) and two-tailed student's t-test (Graphpad Prism for Windows, version 7).

Results and Discussion

Mice treated with PIM1D daily for seven days did not show a significant weight loss (FIG. 9A), and no sign of distress was observed. To gain further information on the potential for PIM 1D toxicity when delivered by IP injection, we analyzed blood chemistry and found that a number of markers sensitive to drug toxicity were unchanged by the initial dosing or even after the last dose of PIM 1D was delivered (FIG. 9B-D). This is a significant improvement over PIM1, where the animals showed marked weight loss and toxic effects following administration of said compound. Therefore, the retention of broad spectrum activity, coupled to a reduced toxicity makes PIM1D a promising antimicrobial compound.

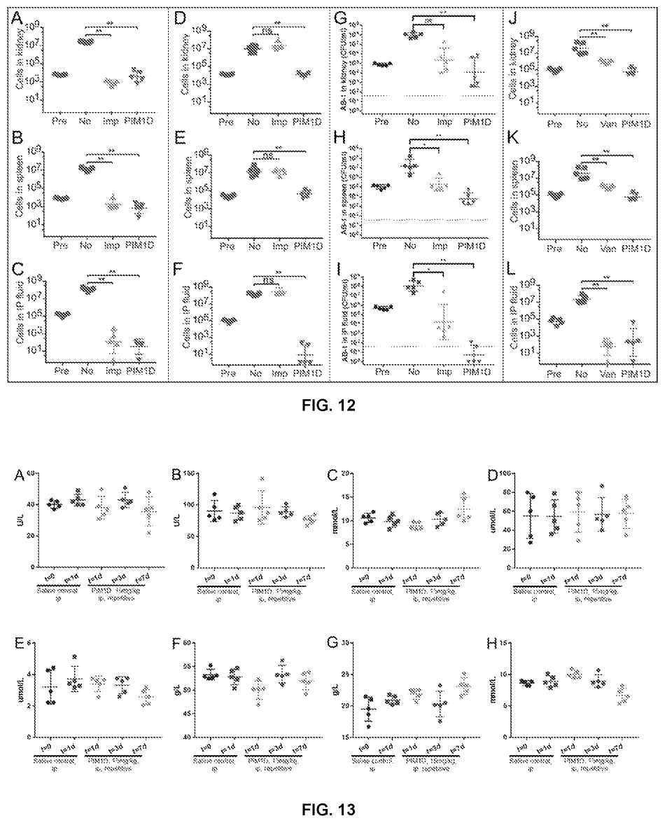

In all septicemia models with different bacterial strains, bacterial cells spread into all organs including kidney, liver and spleen 2 h post-infection when treatment was initiated (see bacterial CFU count in “before treatment” group in FIG. 11A-D. For P. aeruginosa PAO1-induced sepsis shock, PIM1D treatment reduced bacterial burden by more than 3 Log orders in all organs harvested (kidney, liver and spleen) as compared to untreated control (FIG. 12A and FIG. 12A-C), and nearly complete bacterial clearance was observed in peritoneal space, exhibiting similar in vivo efficacy as Imp antibiotic control (FIG. 12C). Moreover, all mice treated with either Imp or PIM1D survived with no signs of distress during the 7 days of monitoring period, while mice without treatment all died (FIG. 11E).

Next, the in vivo efficacy of PIM1D in MDR P. aeruginosa (PAER)-induced peritoneal shock was evaluated. Mice which received a single dose treatment of PIM1D (15mg/kg) 2 h post-infection survived 100%, as compared to zero survival in untreated control or mice receiving the same dose of Imp treatment (FIG. 11F). Also, more than 99.9% bacterial reduction was found in all harvested organs (including kidney, liver and spleen) as compared to untreated control or Imp control, and almost complete bacterial eradication was shown in peritoneal space (FIG. 11B and FIG. 12D-F).

In sepsis model induced by MDR A. baumannii (AB-1), better bacteria reduction was observed for mice treated with single dose of PIM1D (15 mg/kg) than Imp control (15 mg/kg). Around 99.9% bacterial removal was found in harvested organs of mice treated with PIM1D as compared to untreated control, and more than 99.999% bacteria reduction was observed in peritoneal space (FIG. 11C and FIG. 12G-I). Moreover, mice which received PIM1D treatment showed 100% survival as compared to 80% survival in Imp-treated group, and 0% survival in mice group without treatment (FIG. 11G).

Mice blood collected from submandibular vein at day 1, day 3 and day 7 was analyzed using veterinary chemistry analyzer to evaluate ALT, AST and BUN levels, etc. Mice which received saline daily via IP injection were used as control. No significant change was found for ALT and AST levels, which represent the toxicity of liver, and negligible change was observed for BUN level, which represents kidney toxicity, over 7 days (FIG. 13A-H). These results demonstrate that the introduction of degradable moiety successfully reduced the in vivo toxicity of PIM series while maintaining its antibacterial potency in vivo.

### Example 6

### In Vivo Efficacy of PIM1D in Immunosuppressed Mice

Immunosuppression was induced by IP injection of cyclophosphamide (150 mg/kg) at day 4 and cyclophosphamide (100 mg/kg) at day 1 into BALB/c female mice (8-9 weeks of age) before infection was introduced. The animal study protocols were approved by animal ethics and welfare committee at Ningbo University. The mice were infected with methicillin-resistant S. aureus MRSA USA300 by following the protocol in Example 5. Two separate IP injections of 15 mg/kg of antibiotics (PIM1D and vancomycin) were given 2 h and 26 h post-infection. Mice organ harvesting and peritoneal washing were applied 50 h post-infection to determine bacterial burden.

Results and Discussion

The efficacy of PIM1D in MRSA-induced sepsis in immunosuppressed mice was evaluated to further show the broad-spectrum antibacterial activity of PIM1D. More than 99% bacteria reduction was observed in all harvested organs of mice treated with PIM1D as compared to the untreated control, and showed superior bacterial clearance as compared to vancomycin treatment (FIG. 11D and FIG. 12J-L). In the peritoneal space, more than 99.99% bacterial reduction was exhibited, similar to vancomycin treatment control (FIG. 12L). For mice which received either PIM1D or vancomycin treatment, all mice survived in contrast to 0% mice survival in untreated group (FIG. 11H). Therefore, PIM1D spared immunosuppressed mice infected with MRSA USA300 from illness and lowered bacterial burden in affected organs.

### Example 7

### In Vivo Efficacy of PIM1D in Neutropenic Lung Infection Model

To demonstrate the in vivo efficacy in treating distal infection, PIM1D was used to treat neutropenic lung infection model caused by MRSA USA300 and K. pneumoniae (#13883).

Neutropenic Lung Infection Model

Immunosuppression was induced by IP injection of cyclophosphamide (150 mg/kg) at day 4 and cyclophosphamide (100 mg/kg) at day 1 into BALB/c female mice (8-9 weeks of age) before infection was introduced. Lung infection was established by intratracheal delivery of MRSA USA300 or K. pneumoniae (#13883). The infected mice were treated with 20 mg/kg of PIM1D-CA (mixture of PIM1D and citric acid, 1:1 wt %; citric acid is added to minimize the accompanied toxicity) or antibiotics (vancomycin or colistan) via intratracheal delivery 2 h post-infection, while the mice in non-treated group that only received PBS. The mice were monitored for survival over one week. In a separate experiment, mice lungs were harvested 26 h post-infection and homogenized, followed by plating to check the bacterial burden. The animal study protocols were approved by animal ethics and welfare committee at Ningbo University.

Results and Discussion

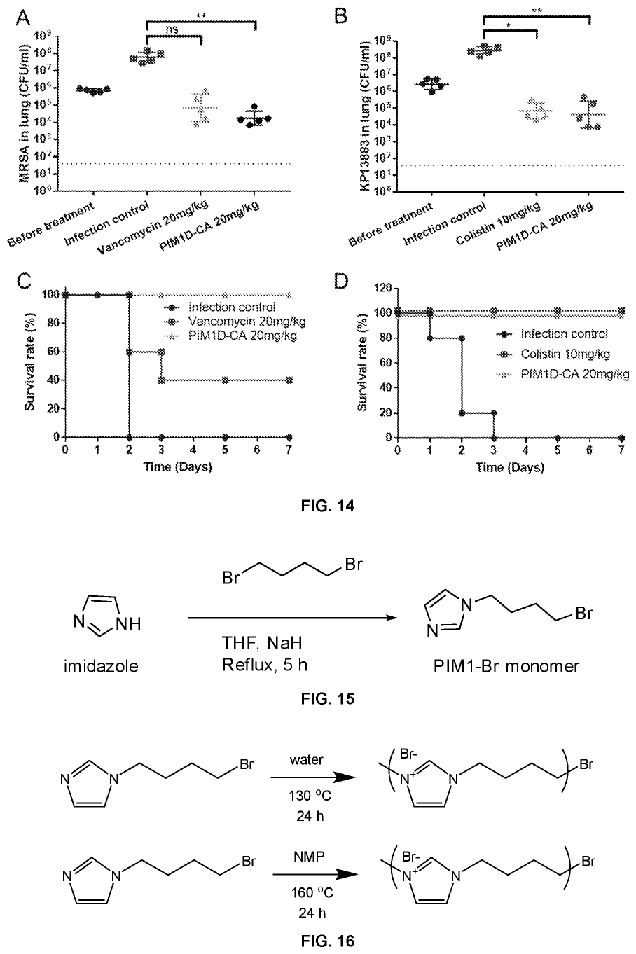

In neutropenic lung infection induced by MRSA, single treatment of 20 mg/kg PIM1D-CA (1:1 wt. % mixture of PIM1D and citric acid) via intratracheal delivery reduced bacterial burden by more than 99.9% efficiency as compared to mice without any treatment (FIG. 14A). In addition, PIM1D-CA treatment was also superior to vancomycin at same treatment dosage. Besides this, infected mice with PIM1D-CA treatment showed 100% survival as compared to zero survival in infection control group and 40% survival for mice which received vancomycin treatment (FIG. 14B), demonstrating PIM1D's excellent activity in treating neutropenic lung infection caused by MRSA.

Considering PIM1D's broad-spectrum antibacterial activity, we also evaluated its efficacy in neutropenic lung infection caused by K. pneumoniae (#13883). Single intratracheal delivery of PIM1D-CA (20 mg/kg) reduced more than 99.9% K. pneumoniae in mice lung compared to infection control (FIG. 14C), as similar in colistin-treated mice. Also, both PIM1D-CA- and colistin-treated mice survived within one monitored week while no mice without treatment survived (FIG. 14D).

Advantages of PIM Over PIM1

The results in Examples 1 to 7 surprisingly demonstrate that PIM1D not only did not show evidence of toxicity but also retained significant antibacterial activity, and showed efficacy in treating murine sepsis infections in vivo. Therefore, together with its good biocompatibility, PIM1D is a superior antibacterial candidate to PIM1.

### Comparative Example 12

### Synthesis of PIM1 Bromide (PIM1-Br) Monomer

Imidazole (10.0 g, 146.9 mmol) was dissolved in THF. NaH (10.6 g, 440.7 mmol) was added portion-wise to the solution at 0° C., and the reaction mixture was allowed to stir for 1 h at room temperature. 1,4-dibromobutane (63.5 g, 294.11 mmol) (2.0 equiv.) was added, and the reaction mixture was heated under reflux (50° C.) for 5 h. (FIG. 15), to afford PIM1-Br monomer as an orange oil (15.1 g, 46%).

¹H NMR (CDCl₃, 300 MHz): δ 3.10-1.23 (m, 4H, —CH₂), 3.43 (t, 2H, —CH₂), 4.06 (t, 2H, —CH₂), 6.90 (s, 2H, imidazole H), 7.04 (s, 2H, imidazole H), 7.49 (s, 1H, imidazole C2-H).

### Comparative Example 13

### Self-Polymerization Route for Preparing PIM1-Br, and the Effect of Reaction Conditions on the Self-Polymerization Reaction

The PIM1-Br monomer prepared in Comparative Example 12 was dissolved in the respective solvent selected from water, NMP and DMF, at a monomer:solvent volume-ratio of 1:3. The polymerization reaction was carried out under vigorous stirring, and heated by immersing the reaction flask in an oil bath. After a predetermined reaction time, the reaction mixture was diluted with DI water, dialyzed (MWCO 1000 Da) in DI water for 3 days, and freeze-dried to obtain the PIM-Br compound (FIG. 16) which was characterized by GPC (Table 11).

Results and Discussion

The effect of different reaction conditions on the self-polymerization reaction of PIM1-Br was investigated using GPC. A summary of the GPC results is provided in Table 11.

TABLE 11
Self-polymerization of PIM1-Br under
different reaction conditions.

| Reaction conditions | |
|---|---|
| Water, 130° C., 24 h | NMP, 150° C., 24 h |

| Sample | Mₙ | M_{w}/Mₙ | Mₙ | M_{w}/Mₙ |
|---|---|---|---|---|
| PIM1-Br | 1779 | 1.21 | 1690 | 1.23 |

### Comparative Example 14

### Antibacterial Efficacy of PIM1-Br

Antibacterial efficacy of PIM1-Br was investigated by following the protocol in Comparative Example 3 to measure the MIC of the compound against different bacteria.

Results and Discussion

TABLE 12
Summary of the antibacterial efficacy of the PIM1-Br.

MIC value (μg/mL)

| | E. coli | S. aureus | P. aeruginosa | MRSA |
|---|---|---|---|---|
| Sample | 8739 | 29213 | PA01 | BAA40 |
| PIM1-Br | 8 | 4 | 4 | 8 |

### Comparative Example 15

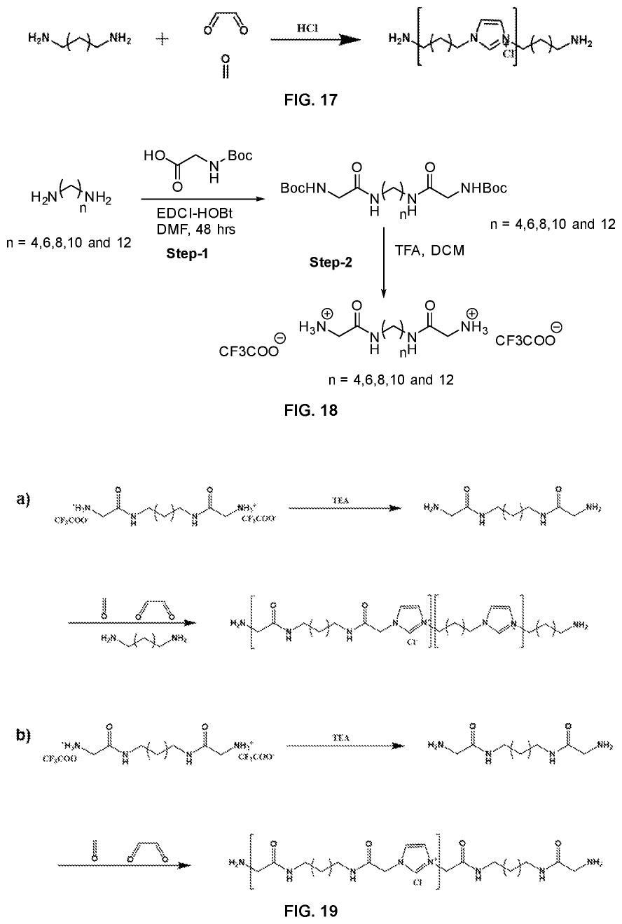

### Synthesis of Nondegradable Main-Chain Cationic PIMs, P(ImC6) and P(ImC8) (FIG. 17)

A compound selected from 1,6-diaminohexane or 1,8-diaminooctane (100 mmol total) in water (30 mL) was introduced into a three-neck flask with a stir bar. HCl (16.7 mL) was slowly added to the reaction mixture. After stirring at room temperature for 30 min, a mixture of 37% formaldehyde (100 mmol) and 40% glyoxal (100 mmol) was introduced. The reaction was refluxed at 100° C. for 12 h, and the color of reaction mixture gradually changed from colorless to yellowish. After removing part of solvent and the unreacted monomers by rotary evaporation, the crude product was dialyzed against acidified water, pH 3-4 (1-KDa-cutoff Spectra/Por®6 dialysis membrane, Repligen, USA) for one day. P(ImC6) and P(ImC8) were characterized by ¹H NMR and GPC analyses (Table 13).

P(ImC6)

¹H NMR (300 MHz, D₂O): δ 8.77 (s, 1H, imidazole-H), 7.48 (s, 2H, imidazole-H), 4.18 (t, 4H), 1.76 (m, 4H), 1.30 (m, 8H).

P(ImC8)

¹H NMR (300 MHz, D₂O): δ 8.77 (s, 1H, imidazole-H), 7.48 (s, 2H, imidazole-H), 4.17 (t, 4H), 1.74 (m, 4H), 1.25 (m, 4H).

List of Abbreviations for Nondegradable PIMs

P(ImC6)—P1

P(ImC8)—P2

### Example 8

### Synthesis of TFA Salt of Diamide Diamine (n =4, 6, 8, 10 and 12) Monomer (FIG. 18)

Diamide Diamine (n=4) TFA Salt

Diamide Diamine (n=4) TFA salt was prepared from diamine B (5.00 g, 56.72 mmol) by following the protocol in Example 1. A while solid was collected and dried to afford a TFA salt of Diamide Diamine (n=4) (48.1%, 11.73 g).

¹H NMR (300 MHz, D₂O): δ 3.24 (s, 4H), 2.68 (s, 4H), 0.96 (s, 4H).

Diamide Diamine (n=6) TFA Salt

Diamide Diamine (n=6) TFA salt was prepared from 1,6-diaminohexane (5.00 g, 43.10 mmol) by following the protocol in Example 1 to afford a TFA salt of Diamide Diamine (n=6) as a white solid (41%, 4.80 g).

¹H NMR (300 MHz, DMSO-D₆): δ 8.35 (t, J=5.4 Hz, 2H), 8.05 (brs, 6H), 3.53 (s, 4H), 3.14 (q, J=6.3 Hz, 4H), 1.54-1.63 (m, 2H).

Diamide Diamine (n=8) TFA Salt

Diamide Diamine (n=8) TFA salt was prepared from 1,8-diaminooctane (2.50 g, 21.55 mmol) by following the protocol in Example 1 to afford a TFA salt of Diamide Diamine (n=8) as a white solid (58.3%, 3.50 g).

¹H NMR (300 MHz, DMSO-D₆): δ 8.34 (t, J=5.4 Hz, 2H), 8.04 (brs, 6H), 3.52 (s, 4H), 3.14 (q, J=6.3 Hz, 4H), 1.42-1.26 (m, 12H).

Diamide Diamine (n=10) TFA Salt

Diamide Diamine (n=10) TFA salt was prepared from 1,10-diaminodecane (2.50 g, 21.55 mmol) by following the protocol in Example 1 to afford a TFA salt of Diamide Diamine (n=10) as an orange solid (46%, 3.80 g).

¹H NMR (300 MHz, DMSO-D₆): δ 8.39 (t, J=5.4 Hz, 2H), 8.12 (brs, 6H), 3.52 (s, 4H), 3.10 (q, J=6.3 Hz, 4H), 1.40-1.24 (m, 16H).

Diamide Diamine (n=12) TFA Salt

Diamide Diamine (n=12) TFA salt was prepared from 1,12-diaminododecane (5.00 g, 43.10 mmol) by following the protocol in Example 1 to afford a TFA salt of Diamide Diamine (n=12) as a white solid (45.3%, 5.50 g).

¹H NMR (300 MHz, DMSO-D₆): δ 8.35 (t, J=5.4 Hz, 2H), 8.05 (brs, 6H), 3.51 (s, 4H), 3.10 (q, J=6.3 Hz, 4H), 1.39-1.23 (m, 22H).

### Example 9

### Synthesis of Degradable Main-Chain Cationic PIMs (P(ImC6-co-ImC6D)-50, P(ImC8-co-ImC8D)-50%), P(ImC6D) and P(ImC8D))

P(ImC6-co-ImC6D)-50% and P(ImC8-co-ImC8D)-50% were synthesized via co-polymerization (FIG. 19a) while P(ImC6D) and P(ImC8D) were synthesized via homopolymerization (FIG. 19b).

P(ImC6-co-ImC6D)-50%

P(ImC6-co-ImC6D)-50% was prepared from Diamide Diamine (n=6) TFA salt and 1,6-diaminohexane by following the protocol in Example 2 with the molar fraction of degradable diamine at 50%, to afford P(ImC6-co-ImC6D)-50%.

¹H NMR (300 MHz, D₂O): δ 8.85 (m, 1H, imidazole-H), 7.50 (m, 2H, imidazole-H), 5.00 (t, 2H), 4.22 (t, 2H), 3.23 (s, 2H), 1.88 (s, 2H), 1.49 (m, 4H).

P(ImC8-co-ImC8D)-50%

P(ImC8-co-ImC8D)-50% was prepared from Diamide Diamine (n=8) TFA salt and 1,8-diaminooctane by following the protocol in Example 2 with the molar fraction of degradable amine at 50%, to afford P(ImC8-co-ImC8D)-50%.

¹H NMR (300 MHz, D₂O): δ 8.85 (m, 1H, imidazole-H), 7.51 (m, 2H, imidazole-H), 5.04 (d, 2H), 4.19 (m, 2H), 3.19 (m, 2H), 1.90 (s, 2H), 1.63-1.39 (m, 8H).

P(ImC6D)

P(ImC6D) was prepared from Diamide Diamine (n=6) TFA salt by following the protocol in Example 2 except no nondegradable amine was added. Following dialysis, P(ImC6D) was obtained.

¹H NMR (300 MHz, D₂O): δ 8.93 (s, 1H, imidazole-H), 7.54 (s, 2H, imidazole-H), 5.08 (s, 4H), 3.24 (s, 4H), 1.56-1.43 (m, 8H).

P(ImC8D)

P(ImC8D) was prepared from Diamide Diamine (n=8) TFA salt following the protocol in Example 2 except no nondegradable amine was added. Following dialysis, P(ImC8D) was obtained.

¹H NMR (300 MHz, D₂O): δ 8.93 (s, 1H, imidazole-H), 7.53 (s, 2H, imidazole-H), 5.00 (s, 4H), 3.24 (s, 4H), 1.51-1.28 (m, 12H).

List of Abbreviations for Degradable PIMs

P(ImC6-co-ImC6D)-50%—P3

P(ImC6D)—P4

P(ImC8-co-ImC8D)-50%—P5

P(ImC8D)—P6

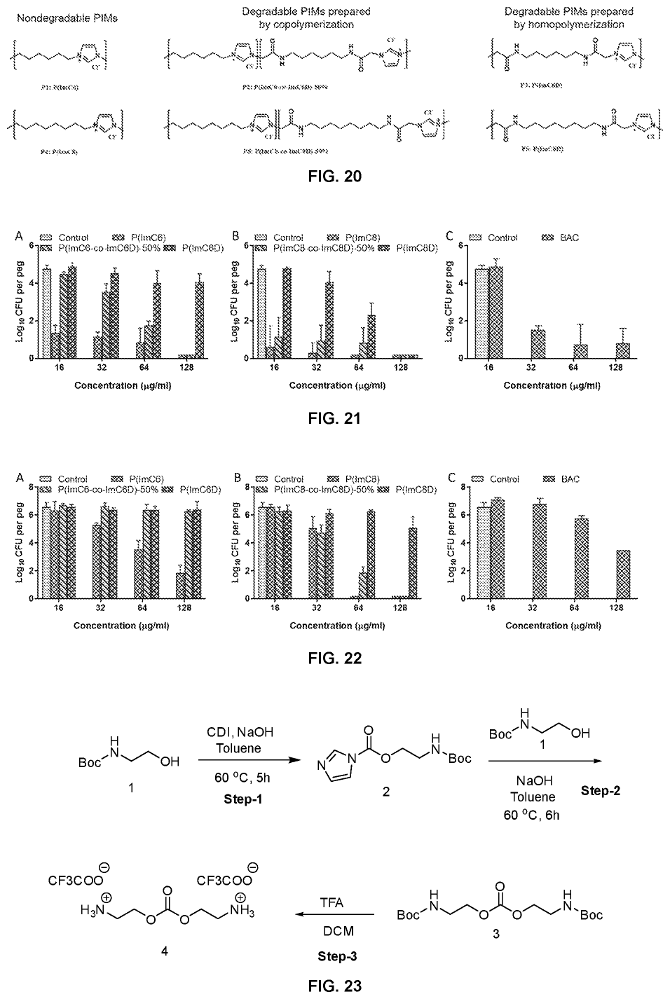

All of the prepared PIMs here and in Comparative Example 15 (FIG. 20) were characterized by ¹H NMR and GPC (Table 13).

TABLE 13
Actual molar fraction of degradable diamine,
Mₙ, M_{w}, and polydispersity (Mₙ/M_{w}) of PIMs.

| | | Actual molar | | | |
|---|---|---|---|---|---|
| | | fraction of | | | |
| | | degradable | | | M_{w}/ |
| Entry | Sample | diamine/% | Mₙ | M_{w} | Mₙ |
| P1 | P(ImC6)-2.9k | 0 | 2900 | 3400 | 1.15 |
| P2 | P(ImC6-co-ImC6D)-50%-2.8k | 58 | 2800 | 3600 | 1.27 |
| P3 | P(ImC6D)-2.1k | 100 | 2100 | 2700 | 1.25 |
| P4 | P(ImC8)-2.7k | 0 | 2700 | 3300 | 1.24 |
| P5 | P(ImC8-co-ImC8D)-50%-2.8k | 52 | 2800 | 3300 | 1.19 |
| P6 | P(ImC8D)-2.5k | 100 | 2500 | 3300 | 1.31 |

### Example 10

### In Vitro Antimicrobial Activity and Cytotoxicity of P1-P6

The antimicrobial activities against planktonic bacteria for the three kinds of PIMs prepared (FIG. 20) were evaluated by following the protocol in Comparative Example 3 to measure their MIC values against Gram-positive bacteria including methicillin-resistant S. aureus BAA39 and S. aureus, and Gram-negative strains, P. aeruginosa O1 and E. coli. Benzalkonium chloride (BAC) was used as a reference. The cytotoxicity of PIMS was tested against mouse embryonic fibroblast 3T3 cells by following the MTT assay protocol in Comparative Example 3.

Results and Discussion

PIMs with higher molar fraction of degradable linker (100%) were less potent than nondegradable PIMs (molar fraction of degradable linker is 0%) in killing bacteria as shown in Table 14. However, this trend was not obvious for PIMs with longer alkyl linker (P4, P5 and P6). Comparing the viability of cells treated with PIMs at different molar fraction of degradable linker (0%, 50%, 100%), we can see an increased trend in biocompatibility with increased fraction, which is an opposite trend to the antimicrobial activities against planktonic bacteria.

TABLE 14
MIC (μg/mL) values of PIMs and BAC
(reference) against a panel of bacteria.

| | | MIC (μg/mL) | | | | |
|---|---|---|---|---|---|---|
| | | MRSA | | | | IC₅₀ |
| Entry | Sample | BAA39 | SA | PAO1 | EC | (μg/mL) |
| P1 | P(ImC6)-2.9k | 1 | 1 | 2 | 4 | 41 |
| P2 | P(ImC6-co-ImC6D)- | 2-4 | 4 | 16 | 8 | 90 |
| | 50%-2.8k | | | | | |
| P3 | P(ImC6D)-2.1k | 8-16 | 8 | 16 | 8 | 128 |
| P4 | P(ImC8)-2.7k | 1-2 | 1-2 | 4 | 2 | 17 |
| P5 | P(ImC8-co-ImC8D)- | 1-2 | 1-2 | 8 | 2 | 17 |
| | 50%-2.8k | | | | | |
| P6 | P(ImC8D)-2.5k | 4 | 4 | 8 | 4 | 46 |
| C3 | Benzalkonium | 1 | 1-2 | 128 | 16 | 10 |
| | chloride | | | | | |

### Example 11

### In Vitro Antibiofilm Activities of P1-P6

MBEC

The MBEC was measured using micro-titre plate-based technology. Briefly, 160 μL of MRSA BAA39 or P. aeruginosa O1 suspension (cell density at ˜10⁷ CFU/mL) was added into the 96-well growth plate covered by a lid containing MBEC pegs. Biofilm were grown on the peg lid after incubation at 37° C. for 24-48 h. After removing planktonic bacteria by washing with PBS twice, the lid with biofilm were transferred into the challenge plate which contained two-fold serial dilutions of one of P1-P6 solution, with total volume of 200 μL in each well. The treatment was performed at room temperature for 4 h. After that, the peg lid was washed again with PBS and transferred to a recovery plate which contained PBS (200 μL) in each well. Biofilm bacteria that survived were dislodged from the peg lid by sonication for 30±5 min, and detached bacteria were then 10-fold serial-diluted in sterile PBS and spread on agar plates. After incubation at 37° C. for 24 h, the colonies were counted.

Results and Discussion

As shown in FIG. 21, the overall antibiofilm efficacy against MRSA BAA39 can be ranked as follows: P(ImC8)>P(ImC8-co-ImC8D)-50%˜P(ImC6)>BAC. Similarly, for P. aeruginosa O1 (FIG. 22), the antibiofilm potency order was: P(ImC8)>P(ImC8-co-ImC8D)-50%>P(ImC6)>BAC.

### Example 12

### Synthesis of Degradable 2+2 Carbonate Monomer (Compound 4)

The synthesis of carbonate monomer (compound 4) involved compounds 1-3 and three steps (FIG. 23). We synthesized imidazole carboxylic esters (compound 2) by reacting CDI with alcohol (compound 1) to obtain compound 2 in good yield. The subsequent carbonyl formation was achieved by treating compound 2 with CDI and compound 1, in the presence of catalytic amount of NaOH to provide the desired boc-protected carbonate (compound 3) in good yield. Boc-deprotection was performed in TFA in DCM to obtain the desired compound 4 in good yield.

### 2-((tert-butoxycarbonyl) amino) ethyl 1H-imidazole-1-carboxylate (Compound 2)

In a 250 mL round-bottom flask fitted with a dry N₂ inlet and magnetic stirrer, dry toluene (150 mL) and 1,1′-carbonyldiimidazole (CDI, 10.0 g, 0.0310 mol) were added, followed by tert-butyl (2-hydroxyethyl)carbamate (compound 1, 5.0 g, 0.0198 mol), and KOH (5.2 mg, 0.003 mol). The mixture was heated at 60° C. with stirring for 4 h. The formation of a clear solution was observed. The reaction mixture was cooled to room temperature. The solution was concentrated in vacuo, dissolved in DCM (200 mL), then washed thrice with water (3×50 mL). The solution was dried with anhydrous Na₂SO₄ and concentrated in vacuo to give compound 2 as a white solid (5.1 g, 62.1%).

¹H NMR (300 MHz, DMSO-D₆): δ 8.15 (s, 1H), 7.44 (s, 1H), 7.07 (s, 1H), 4.91 (brs, 1H), 4.47 (t, J=5.2 Hz, 2H), 3.52 (q, J=6.3 Hz, 2H), 1.44 (s, 9H).

### Di-tert-butyl ((carbonvlbis(oxv)bis(ethane-2,1-diyl) dicarbamate (Compound 3)

In a 250 mL round-bottom flask fitted with a dry N₂ inlet and magnetic stirrer, dry toluene (150 mL) and CDI (6.3 g, 0.0389 mol) were added, followed by compound 2 (5.0 g, 0.019 mol), compound 1 (3.17 g, 0.0195 mol), and KOH (5.17 mg, 0.003 mol) The mixture was heated at 60° C. with stirring for 18 h. The formation of a clear solution was observed. The reaction mixture was cooled to room temperature. The solution was concentrated in vacuo, dissolved in DCM (200 mL), and washed thrice with water (3×50 mL). The solution was dried with anhydrous Na₂SO₄ and concentrated in vacuo. The resulting crude product was purified by column chromatography (EtOAc:Hexanes 3:7) to afford compound 3 as a white solid (4.80 g, 58.8%).

¹H NMR (300 MHz, DMSO-D₆): δ 5.21 (brs, 2H), 4.29 (t, J=5.1 Hz, 4H) 3.33 (s, 4H), 1.25 (s, 18H).

### 2,2′-(carbonylbis(oxy)diethanaminium 2,2,2-trifluoroacetate (Compound 4)

In a 100 mL round-bottom flask fitted with a dry N₂ inlet and magnetic stirrer, compound 3 (4.0 g, 0.0389 mol) was dissolved in dry DCM (50 mL) and TFA (6 mL, excess) was added. The reaction mixture was stirred at room temperature for 18 h. Then, the reaction mixture was concentrated under reduced pressure to give carbonate monomer 4 as a white solid (3.60 g, 75%).

¹H NMR (300 MHz, D₂O): δ 4.34 (t, J=5.1 Hz, 4H), 3.27-3.24 (m, 4H). ¹³C NMR (75 MHz, D₂O): δ 166.14, 159.58 (—CO—CF₃), 159.16 (—CO—CF₃), 158.74 (—CO—CF₃), 158.32 (—CO—CF₃), 154.56 (CO—O), 123.24 (—CF₃), 119.29 (—CF₃), 115.33 (—CF₃), 111.38 (—CF₃), 64.26, 38.23.

### Example 13

### Synthesis of Biodegradable PIM D2 with Carbonate Linker

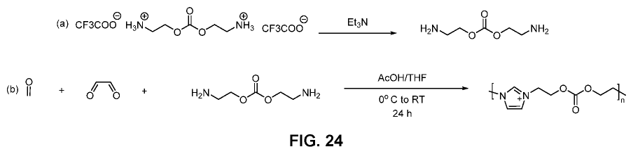

PIM D2-1-8 were prepared from compound 4 (FIG. 24) by following the protocol in Example 2, and controlling the stoichiometric ratios and concentration of starting materials (Table 15).

¹H NMR (300 MHz, D₂O): δ 8.85 (m, 1H, imidazole-H), 7.50 (m, 2H, imidazole-H), 4.47 (s, 4H). ¹³C NMR (75MHz, D₂O): δ 154.29, 136.64, 122.96, 66.25, 48.31.

TABLE 15
Summary of polymerization condition and the molecular weight
of carbonate-incorporated biodegradable PIMs (PIM D2).

| | Molar | | Reaction | | Mₙ, GPC | | MIC | |
|---|---|---|---|---|---|---|---|---|
| Samples | Ratio^{a} | Concentration^{b} | Time | Temperature | (g/mol) | PDI | S. aureus^{c} | PAO1 |
| PIM D2-1 | 1:1 | 0.4 mmol | 24 h | 0-RT | 966 | 1.03 | >256 | >256 |
| PIM D2-3 | 0.9:1 | 0.4 mmol | 24 h | 0-RT | 1249 | 1.04 | >256 | >256 |
| PIM D2-4 | 0.9:1 | 1.2 mmol | 24 h | 0-RT | 1262 | 1.05 | >256 | 256 |
| PIM D2-5 | 0.8:1 | 0.4 mmol | 24 h | 0-RT | 1522 | 1.08 | 32 | 32 |
| PIM D2-6 | 0.7:1 | 0.4 mmol | 24 h | 0-RT | 1426 | 1.09 | 32 | 32 |
| PIM D2-7 | 0.6:1 | 0.4 mmol | 24 h | 0-RT | 1376 | 1.08 | >256 | >256 |
| PIM D2-8 | 0.5:1 | 0.4 mmol | 24 h | 0-RT | 1347 | 1.08 | >256 | >256 |

^{a}Molar Ratio is the ratio of diamine to aldehyde;
^{b}Concentration is the concentration of aldehyde;
^{c}S. aureus is S. aureus 29213.

From Table 15, we can see that the concentration of diamine only had a minor effect on the molecular weight of the polymers but the stoichiometric ratios of diamine to aldehyde showed a significant effect on the molecular weight of the polymers. The highest molecular weight obtained is PIM D2-5 with a molecular weight of 1522 g/mol and a narrow polydispersity of 1.08. The chemical structures of carbonate-incorporated biodegradable PIMs were further verified with both ¹H NMR and ¹³0 NMR spectra.

### Example 14

### Stepwise Synthesis of Degradable Hexaimidazoliums (OIM1D-3C-6 and OIM1D-3C-8)

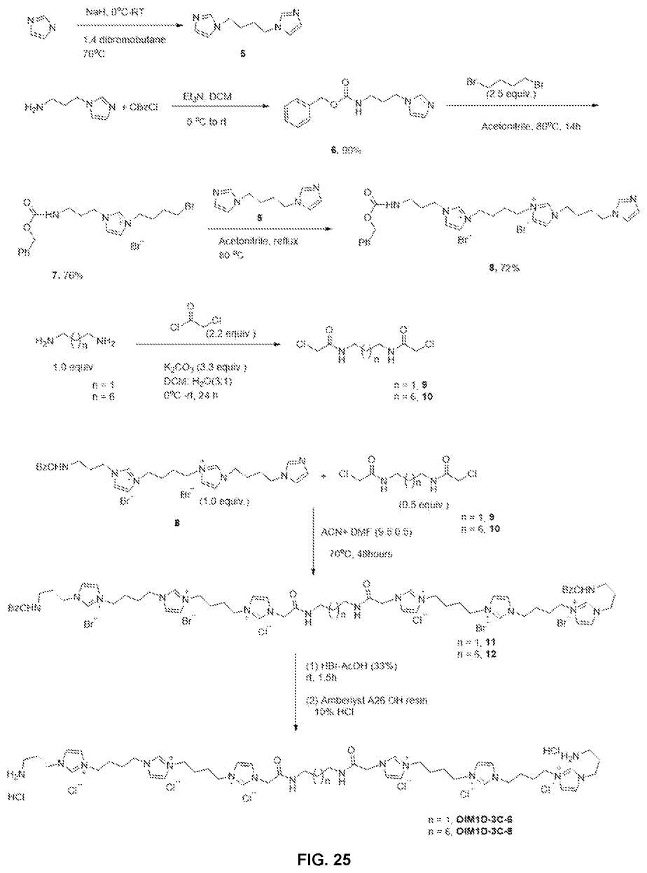

Considering good antibacterial activity and biocompatibility of PIM1D, stepwise synthesis to make oligoimidazoliums with biodegradable amide linker and well-defined molecular weight was explored. Imidazoliums with three repeat units were prepared via a step by step method, which were joined together using a N,N′-(alkane-1,3-diyl)bis(2-chloroacetamide) linker to obtain the final degradable compounds, which are termed OIM1D-30-6 and OIM1D-30-8 for the degradable linker with three carbons and eight carbons in the alkyl chain, respectively. The synthesis was achieved in six steps (FIG. 25) and required eight intermediate compounds (compounds 5-12) to afford the final degradable oligoimidazoliums (OIM1D-30-6 and OIM1D-30-8). The compounds were characterized by NMR and MALDI-TOF, where applicable.

### 1,4-di(1H-imidazol-1-yl)butane (Compound 5)

Compound 5 was prepared from imidazole (4.00 g, 0.058 mol 1 1 equiv.) by following the protocol in Comparative Example 12 except the reaction mixture was heated under reflux (70° C.) overnight, and the product was purified by extraction with MeOH. The MeOH phase was washed thrice with hexane, and white solid crystals of compound 5 was obtained by rotary evaporation (10.2 g, 92%).

¹H NMR (300 MHz, DMSO-d₆) δ 7.61 (s, 2H), 7.14 (brs, 2H), 6.89 (brs, 2H), 3.98-3.73 (m, 4H), 1.64-1.59 (m, 4H). MALDI-TOF (CHCA matrix, Reflector mode) C₁₀H₁₄N₄: calc. 190.1218 (M); found 191.1296 (M+H).

Compound 6

Triethylamine (Et₃N) (1.2 equiv., 10.6 g, 0.105 mol) was added to a stirred solution of aminopropyl imidazole (1.0 equiv., 11.0 g, 0.088 mol) in DCM (110 mL) at 0° C. CBzCI (1.1 equiv., 16.5 g, 0.096 mol) was added slowly via a syringe over a period of 10 min. The reaction mixture was allowed to stir and warm to room temperature overnight. The reaction was transferred to a separatory funnel, and the organic layer was extracted with 0.2 M HCl (100 mL), followed by four consecutive extractions with water (100 mL). The organic layer was dried over anhydrous Na₂SO₄, concentrated via rotary evaporation and subjected to silica gel chromatography to afford compound 6 (20.5 g, 90%).

¹H NMR (300 MHz, DMSO-d₆) δ 7.63 (s, 1H), 7.50-7.24 (m, 6H), 7.17 (s, 1H), 6.90 (s, 1H), 5.04 (s, 2H), 3.97 (t, J=6.9 Hz, 2H), 2.98 (q, J=6.3 Hz, 2H), 1.84 (p, J=6.7 Hz, 2H). ¹³C NMR (75 MHz, DMSO-d₆) δ 156.1, 137.2, 137.1, 128.3, 127.7, 119.3, 65.3, 43.4, 37.4, 31.0.

Compound 7

1,4-dibromobutane (4.5 mL, 0.0375 mol, 2.5 equiv.) was added to a stirred solution of compound 6 (3.00 g, 0.0115 mol, 1.0 equiv) in dry ACN (10 mL) under argon atmosphere. The reaction mixture was heated at 70° C. for 14 h, then cooled to room temperature. The solvent was removed by rotary evaporation under vacuum and subjected to silica gel chromatography by eluting EtOAc to 15% MeOH/EtOAc to afford compound 7 as a white syrup (4.10 g, 76%).

¹H NMR (300 MHz, DMSO-d₆) δ 9.39 (s, 1H), 7.88 (d, J=3.4 Hz, 2H), 7.58-7.21 (m, 6H), 5.02 (s, 2H), 4.24 (q, J=7.2 Hz, 4H), 3.56 (t, J=6.4 Hz, 2H), 3.02 (q, J=6.0 Hz, 2H), 2.05-1.86 (m, 4H), 1.86-1.72 (m, 2H). ¹³C NMR (75 MHz, DMSO-d₆) δ 156.2, 137.0, 136.2, 128.3, 127.76, 127.70, 122.45, 122.40, 65.3, 47.9, 46.5, 36.9, 34.1, 29.7, 28.7, 28.1. MALDI-TOF (CHCA matrix, Reflector mode) C₁₈H₂₅Br₂N₃O₂: calc. 473.0314 (M); found 394.1405 (M-Br).

Compound 8

Compound 5 (1.80 g, 0.009 mol, 1.5 equiv.) was added to a stirred solution of compound 7 (3.0 g, 0.006 mol, 1.0 equiv.) in dry ACN (10 mL), and the resulting mixture was heated at 70° C. under argon atmosphere overnight. After completion of reaction monitored by TLC, the solvent was removed under vacuum, and the obtained mixture was subjected to flash silica gel (100-200 mesh) column chromatography (mobile phase EtOAc to MeOH; 10 to 50%) to obtain compound 8 as a hygroscopic white solid (3.00 g, 72%).

¹H NMR (300 MHz, DMSO-d₆) δ 9.35 (t, J=24.3 Hz, 2H), 7.88-7.78 (m, 4H), 7.72 (s, 1H), 7.48-7.28 (m, 6H), 7.20 (s, 1H), 6.92 (s, 1H), 5.02 (s, 2H), 4.20 (t, J=6.9 Hz, 8H), 4.02 (t, J=6.5 Hz, 2H), 3.05-2.99 (m, 2H), 2.02-1.88 (m, 2H), 1.80-1.72 (m, 8H). ¹³C NMR (75 MHz, DMSO-d₆) δ 155.0, 136.0, 135.8, 135.1, 134.7, 127.2, 127.0, 126.68, 126.60, 121.3, 121.2, 118.1, 64.0, 47.0, 46.9, 45.4, 44.0, 43.8, 35.7, 28.5, 26.0, 25.3, 24.8. MALDI-TOF (CHCA matrix, Reflector mode) C₂₈H₃₉Br₂N₇O₂calc. 663.1532 (M); found (M-2Br-H) 504.3814.

Compound 9

To a solution of K₂CO₃ (33 mmol, 3.3 equiv.) in water/DCM (1:3, 18 mL) at 0° C., 1,3-diaminopropane (10.0 g, 1 equiv.) was added. The resulting mixture was allowed to cool down before chloroacetyl chloride (22 mmol, 2.2 equiv.) was added dropwise over a 1-h period at 0° C. After complete addition, the ice bath was removed and the mixture was allowed to stir at room temperature overnight. The desired product was extracted thrice with DCM. Subsequently, the organic layer was washed with brine, dried over Na₂SO₄, filtered and concentrated under reduced pressure to afford compound 9 (82%, 24.5 g).

¹H NMR (DMSO-d₆) δ 8.59 (s, 2H), 4.05 (s, 4H), 3.09 (t, 4H), 1.55-1.62 (m, 2H).

Compound 10

Compound 10 was prepared from 1,8-diaminooctane (1 equiv.) based on the protocol for compound 9.

Compound 11

To a stirred solution of compound 8 (1.0 equiv.) in ACN:DMF (9:1) at room temperature, compound 9 (0.5 equiv.) was added, then heated at 80° C. for 48 h. The reaction mixture was cooled down to room temperature, and the obtained precipitate was filtered and collected as a hygroscopic gummy compound which was further washed with ACN thrice, and freeze dried to afford a crude mixture of compound 11 and impurities.

¹H NMR δ (D₂O) 8.79 (s, 2H), 8.72 (s, 2H), 8.62 (s, 2H), 7.46-7.31 (m, 24H), 4.96 (s, 8H), 4.24-4.11 (m, 20H), 3.08-3.05 (m, H), 3.05-3.03 (m, 4H), 1.82-1.64 (m, 22H). ¹³C NMR (75 MHz, DMSO-d₆) δ 165.5, 156.8, 132.5, 131.9, 127.1, 126.3, 122.4, 121.15, 121.12, 65.4, 48.1, 47.4, 46.8, 35.7, 27.5, 26.4, 2.8.

Compound 12

Compound 12 was prepared from compound 8 and 10 based on the protocol for compound 11.

¹H NMR δ (DMSO-d₆) 9.56 (s, 2H), 9.47 (s, 2H), 9.37 (s, 2H), 8.65 (s, 2H), 7.88-7.84 (brs, 12H), 7.49 (s, 2H), 7.10-7.32 (m, 10H), 5.05 (s, 4H), 5.02 (s, H), 4.08-4.06 (m, 20H), 3.04-3.01 (m, 8H), 1.97-1.82 (m, 20H), 1.26-1.15 (m, 14H).

OIM1D-3C-6

Compound 11 was dissolved in a solution of HBr in AcOH (33%), and the resulting mixture was stirred at room temperature for 3 h. The addition of EtOAc (2 mL) caused the amine salt to precipitate. The solvent was drawn off, and the resulting residue was retained. The resulting compound was dissolved in water (50-60 mM), and was passed through a column containing chloride-loaded Amberlyst® A-26 (OH— form). The column was further washed with water until complete isolation of the compound, which was then concentrated under vacuum. The obtained material was diluted with water and was dialysed (Mw-CO 500-1000 D) against acidified water (1 mL) for 1 day, with the acidified water changed 6-7 times. The solution in the dialysis bag was decanted into Falcon tube and freeze-dried to afford OIM1D-3C-6 (approximately 30%).

¹H NMR δ (D₂O) 8.79 (s, 4H), 8.74 (s, 2H), 7.46-7.41 (m, 12H), 4.96 (s, 4H), 4.19-4.15 (m, 20H), 3.17 (s, 4H), 2.90 (s, 4H), 2.18 (m, 4H), 1.88-1.65 (m, 18H).

OIM1D-30-8OIM1D-3C-8 was prepared from compound 12 based on the protocol for OIM1D-3C-6.

¹H NMR δ (D₂O) 8.80 (s, 4H), 8.75 (s, 2H), 7.47-7.40 (m, 12H), 4.94 (s, 4H), 4.24-4.16 (m, 20H), 3.03 (t, 4H), 2.90 (t, 4H), 2.19-2.16 (m, 4H), 1.83 (brs, 16H), 1.42-1.40 (m, 4H), 1.48-1.46 (m, 8H).

### Example 15

### In Vitro Biological Profile of Degradable OIMID-3C-6 and OIMID-3C-8

The in vitro biological profile of OIM1D-30-6 and OIM1D-30-8 was evaluated using MIC and MTT experiments described in Comparative Example 3.

Results and Discussion

OIM1D-3C-6 and OIM1D-30-8 showed good antibacterial activity towards both S. aureus and methicillin-resistant S. aureus, as well as E. coli, with MIC₉₀ in the range of 2-16 μg/mL (Table 16). OIM1D-3C-6 showed decreased antibacterial potency against P. aeruginosa PAO1, with MIC₉₀ of 128 μg/mL. Both OIM1D-30-6 and showed good biocompatibility with IC₅₀ more than 1024 μg/mL, as determined by MTT test using 3T3 fibroblast cells.

TABLE 16
MIC₉₀ of OIM1D-3C-6 and OMI1D-3C-8 against
pathogens and human cell lines.

Concentration (μg/mL)

| | | |
|---|---|---|
| Organism and genotype | OIM1D-3C-6 | OIM1D-3C-8 |

Pathogenic bacteria (MIC₉₀)

| | | |
|---|---|---|
| S. aureus 29213 | 2 | 2 |
| S. aureus BAA40 (MRSA) | 4 | 8 |
| E. coli 8739 | 16 | 16 |
| P. aeruginosa PAO1 | 128 | ND |

Human cell line (IC₅₀)

| | | |
|---|---|---|
| Human fibroblast (3T3) | >1024 | >1024 |

ND, not determined, no data.
MRSA, Methicillin-resistant staphylococcus aureus.

Therefore, by tuning the degradable linker chain, degradable functional group, imidazolium repeating units and end group, a library of biodegradable oligoimidazoliums with versatile functions could be built. This would be a good candidate for mechanistic studies, degradation rate studies, pharmacokinetic and pharmacodynamic studies in animal models.

### Example 16

### In Vivo Study of Degradable OIM1D-3C-6 and OIM1D-3C-8

The in vivo efficacy of OIM1D-3C-6 and OIM1D-3C-8 was evaluated using the neutropenic lung infection model described in Example 7, while their in vivo intranasal toxicity was determined as described below.

In Vivo Intranasal Toxicity

20 mg/kg of OIM1D-3C-8 and OIM1D-3C-8/OIM1D-3C-6 mixture were intranasally delivered into randomly grouped mice (ICR, female). The mice weight and condition were monitored daily till 7 days post-compound delivery.

Results and Discussion

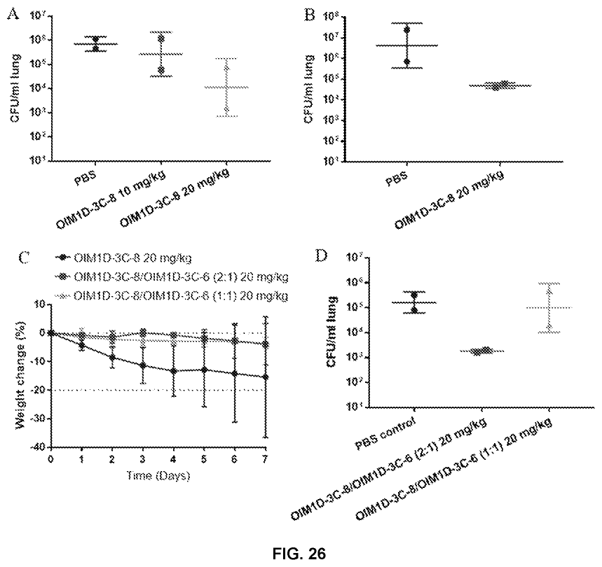

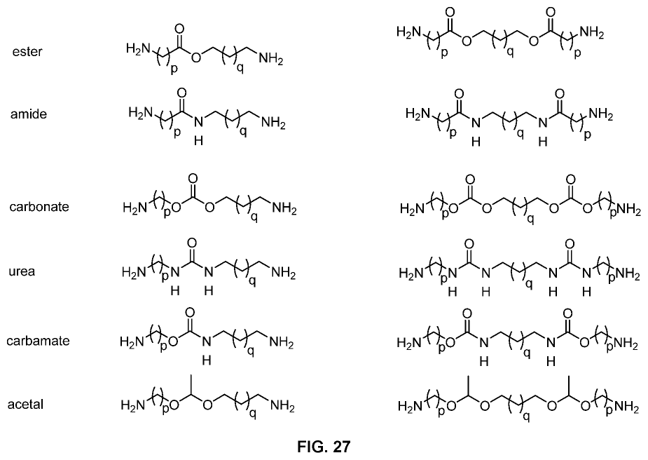

The results showed that with 10 mg/kg of OIM1D-3C-8, the bacteria burden was reduced by 60%, while 20 mg/kg OIM1D-3C-8 reduced bacterial loads by about 2 Log order (FIG. 26A), demonstrating the efficacy of OIM1D-3C-8 in reducing bacterial burden in lung infection model. In the neutropenic lung infection induced by methicillin-resistant S. aureus, about 2 Log order reduction in bacterial loads was also observed (FIG. 26B), demonstrating the efficacy of OIM1D-3C-8 in fighting against Gram-positive bacterial infection. Then, we investigated the toxicity of OIM1D-3C-8 by intranasal delivery of OIM1D-3C-8 (20 mg/kg), followed by weight monitoring. The results showed that this led to a gradual weight decrease over time (FIG. 26C).

To reduce the in vivo toxicity of OIM1D-3C-8, we mixed OIM1D-3C-8 with OIM1D-3C-6 at 2:1 and 1:1 weight ratio. With this mixture, the toxicity was successfully reduced, and negligible weight loss was observed over time (FIG. 26C). Then, we evaluated the efficacy of these two mixtures, and found that for the OIM1D-3C-8/OIM1D-3C-6 (2:1 wt. %) mixture, about two Log order reduction in bacterial loads was observed in lung infection induced by MDR K. pneumonia (FIG. 26D), which is similar to OIM1D-3C-8. Taken together, we had demonstrated that the OIM1D-3C-8/OIM1D-3C-6 (2:1 wt. %) mixture had good in vivo efficacy with limited toxicity, suggesting its potential as a therapeutic agent in MDR bacterial infection.

1. A polymer or oligomer or a pharmaceutically acceptable solvate thereof comprising a first repeating unit comprising an imidazolium group and a biodegradable chain connected to an adjacent repeating unit.

2. The polymer or oligomer according to claim 1, wherein the only repeating unit is the first repeating unit.

3. The polymer or oligomer according to claim 1, wherein the polymer or oligomer further comprises a second repeating unit comprising an imidazolium group and a non-biodegradable alkyl chain or a further biodegradable alkyl chain connected to an adjacent repeating unit.

4. The polymer or oligomer according to claim 3, wherein one or more of the following apply:
(a) the polymer or oligomer comprises from 1 to 75 mol % of the first repeating unit; and
(b) the repeating units of the polymer or oligomer are randomly distributed or the repeating units are formed as blocks.

5. The polymer or oligomer according to claim 1, wherein the biodegradable chain in the first repeating unit comprises one or more biodegradable functional groups, where the one or more biodegradable functional groups are selected from one or more of the group consisting of urea, carbamate, acetal, amide, ester, carbonate ester, urethane, disulfide, anhydride, and hydrazone.

6. The polymer or oligomer according to claim 1, wherein the number average molecular weight is from 800 to 10,000 Daltons.

7. The polymer or oligomer according to claim 1, wherein the polymer or oligomer has the formula I: 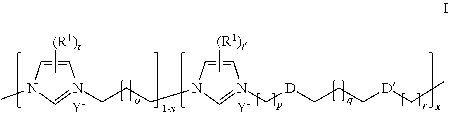
wherein:
x is from 0.01 to 1.0;
Y⁻ is a counterion;
o is from 0 to 10;
p is from 1 to 12;
q is from 0 to 14;
r is from 0 to 12;
D is a biodegradable functional group;
D′ is a biodegradable functional group or a bond;
each R¹ is a branched or unbranched C₁₋₃ alkyl or derivatives thereof;
each t is 0, 1 or 2;
each t′ is 0, 1 or 2;
each R² is a branched or unbranched C₁₋₃ alkyl or derivatives thereof;
or a pharmaceutically acceptable solvate thereof.

8. The polymer or oligomer according to claim 7, wherein one or more of the following apply:
(bi) each D is selected from urea, carbamate, acetal, amide, ester, carbonate ester, urethane, disulfide, anhydride, and hydrazone;
(bii) each D′ is selected from a bond, urea, carbamate, acetal, amide, ester, carbonate ester, urethane, disulfide, anhydride, and hydrazone,
(biii) Y⁻ is selected from one or more of the group consisting of halo, acetate, phosphate, sulfonate, and bis((trisfluoromethyl)sulfonyl)imide (N(Tf)₂⁻), optionally wherein Y⁻ is selected from one or more of the group consisting of chloro, acetate, phosphate, sulfonate, and bis((trisfluoromethyl)sulfonyl)imide (N(Tf)₂⁻);
(biv) x is from 0.01 to 1.0, such as from 0.025 to 0.75, such as from 0.05 to 0.6, such as from 0.1 to 0.5, such as from 0.2 to 0.3;
(bv) t and t′ are 0;
(bvi) p is from 1 to 6; and
(bvii) r is from 1 to 6.

9. The polymer or oligomer according to claim 1, wherein the polymer is selected from the group consisting of:

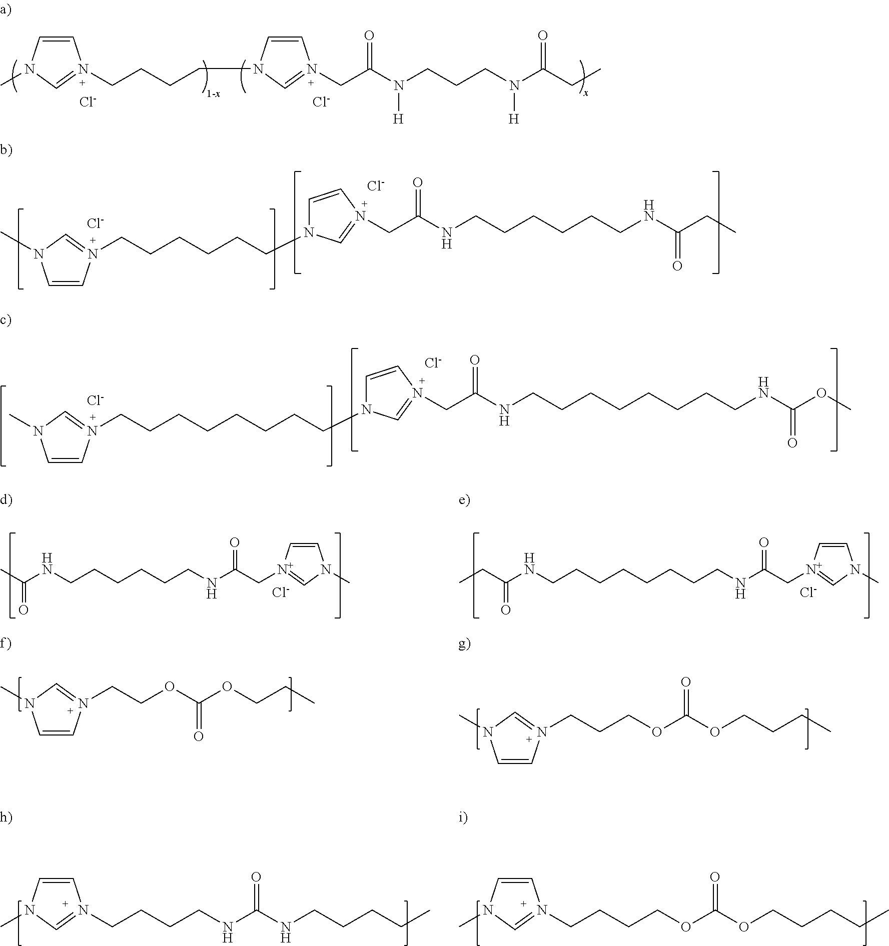

10. A molecule or a pharmaceutically acceptable solvate thereof comprising:
a first block of oligomeric repeating units, where each repeating unit comprises an imidazolium group and a non-biodegradable alkyl chain connected to an adjacent repeating unit;
a second block of oligomeric repeating units, where each repeating unit comprises an imidazolium group and a non-biodegradable alkyl chain connected to an adjacent repeating unit; and
a linking group connecting the first block and the second block together, wherein the linking group comprises one or more biodegradable functional groups.

11. The molecule according to claim 10, wherein the one or more biodegradable functional groups are selected from one or more of the group consisting of urea, carbamate, acetal, amide, ester, carbonate ester, urethane, disulfide, anhydride, and hydrazone.

12. The molecule according to claim 10 wherein, the molecular weight is from 1,000 Daltons to 5,000 Daltons, optionally wherein the molecular wcight is from 1,000 Daltons to 1,000 Daltons.

13. The molecule according to claim 10, wherein the molecule has the formula II: 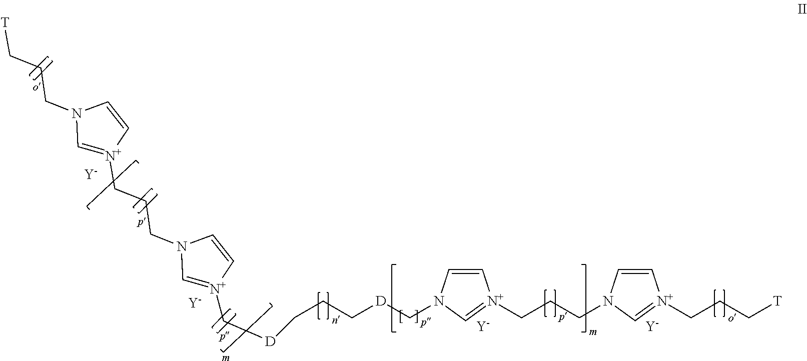
wherein:
each m is independently from 1 to 8;
each Y⁻ is a counterion;
n′ is from 0 to 12;
each o′ is independently selected from 0 to 20;
each p′ is independently selected from 0 to 12;
each p″ is independently selected from 0 to 12;
each T is independently a terminal functional group selected from amine, ammonium, guanidinium, bisguanidinium, alkyl, and aryl;
each D is a biodegradable functional group, or a pharmaceutically acceptable solvate thereof.

14. The molecule according to claim 13, wherein one or more of the following apply:
(di) each D is independently selected from urea, carbamate, acetal, amide, ester, carbonate ester, urethane, disulfide, anhydride, and hydrazone,
(dii) Y⁻ is selected from one or more of the group consisting of halo, acetate, phosphate, sulfonate, and bis((trisfluoromethyl)sulfonyl)imide (N(Tf)₂⁻); and
(dii) p″ is 0 to 6.

15. The molecule according to claim 10, wherein the molecule is selected from the group consisting of:

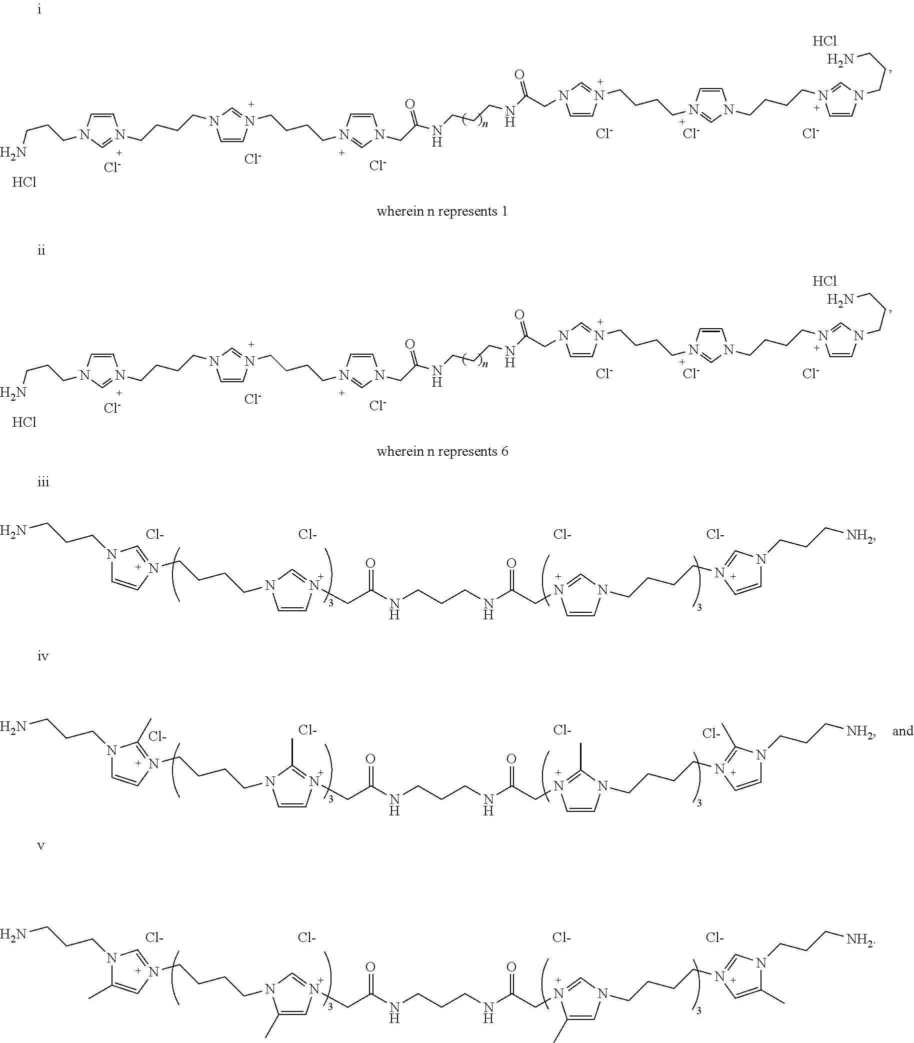

16. (canceled)

17. (canceled)

18. (canceled)

19. A method of treatment of a disease comprising a microbial infection comprising the step of administering to a subject in need thereof a therapeutically effective amount of a polymer or oligomer or a pharmaceutically acceptable solvate thereof according to claim 1.

20. The method according to claim 19, wherein the microbial infection is an infected wound or cystic fibrosis.

21. An antiseptic formulation comprising a polymer or oligomer or a pharmaceutically acceptable solvate thereof according to claim 1.

22. An article having a surface, wherein the surface is coated with a polymer or oligomer or a pharmaceutically acceptable solvate thereof according to claim 1 to provide said surface of the article with antimicrobial properties.

23. A method of treatment of a disease comprising a microbial infection comprising the step of administering to a subject in need thereof a therapeutically effective amount of a molecule or a pharmaceutically acceptable solvate thereof according to claim 10.
